SEQUENCE LISTING (36 sequences)

SEQ ID NO: 1 - protein, 187 aa, Artificial Sequence
  Description of Artificial Sequence Synthetic polypeptide
  IFNbeta
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 2 - protein, 149 aa, Artificial Sequence
  Description of Artificial Sequence Synthetic polypeptide
  CD40L soluble form
MQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 3 - protein, 154 aa, Artificial Sequence
  Description of Artificial Sequence Synthetic polypeptide
  TWEAK soluble form
MKGRKTRARRAIAAHYEVHPRPGQDGAQAGVDGTVSGWEEARINSSSPLRYNRQIGEFIVTRAGLYYLYCQVHFDEGKAVYLKLDLLVDGVLALRCLEEFSATAASSLGPQLRLCQVSGLLALRPGSSLRIRTLPWAHLKAAPFLTYFGLFQVH

SEQ ID NO: 4 - protein, 167 aa, Artificial Sequence
  Description of Artificial Sequence Synthetic polypeptide
  LIGHT soluble form
DGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 5 - protein, 228 aa, Artificial Sequence
  Description of Artificial Sequence Synthetic polypeptide
  Peptide linker IgG1 Fc Domain
DDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 13 aa, Artificial Sequence
  Description of Artificial Sequence Synthetic peptide
  Peptide Gly-Ser-Thr linker
SGGTSGSTSGTGS

SEQ ID NO: 7 - protein, 14 aa, Artificial Sequence
  Description of Artificial Sequence Synthetic peptide
  Peptide Gly-Ser-Thr linker
SGGTSGSTSGTGST

SEQ ID NO: 8 - protein, 34 aa, Artificial Sequence
  Description of Artificial Sequence Synthetic polypeptide
  Peptide linker incorporating a multimerization leucine zipper domain
VSSIEKKIEEITSQIIQISNEITLIRNEIAQIKQ

SEQ ID NO: 9 - protein, 591 aa, Artificial Sequence
  Description of Artificial Sequence Synthetic polypeptide
  Human IFNbeta-IgG1 FC-CD40L
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNSGGTSGSTSGTGSDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGTSGSTSGTGSTMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 10 - protein, 350 aa, Artificial Sequence
  Description of Artificial Sequence Synthetic polypeptide
  Human IFNbeta-Linker-CD40L
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNSGGTSGSTSGTGSTMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 11 - protein, 384 aa, Artificial Sequence
  Description of Artificial Sequence Synthetic polypeptide
  Human IFNbeta-LZ-CD40L
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNSGGTSGSTSGTGSTVSSIEKKIEEITSQIIQISNEITLIRNEIAQIKQMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 12 - protein, 596 aa, Artificial Sequence
  Description of Artificial Sequence Synthetic polypeptide
  Human IFNbeta-IgG1 FC-TWEAK
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNSGGTSGSTSGTGSDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGTSGSTSGTGSTMKGRKTRARRAIAAHYEVHPRPGQDGAQAGVDGTVSGWEEARINSSSPLRYNRQIGEFIVTRAGLYYLYCQVHFDEGKAVYLKLDLLVDGVLALRCLEEFSATAASSLGPQLRLCQVSGLLALRPGSSLRIRTLPWAHLKAAPFLTYFGLFQVH

SEQ ID NO: 13 - protein, 355 aa, Artificial Sequence
  Description of Artificial Sequence Synthetic polypeptide
  Human IFNbeta-Linker-TWEAK
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNSGGTSGSTSGTGSTMKGRKTRARRAIAAHYEVHPRPGQDGAQAGVDGTVSGWEEARINSSSPLRYNRQIGEFIVTRAGLYYLYCQVHFDEGKAVYLKLDLLVDGVLALRCLEEFSATAASSLGPQLRLCQVSGLLALRPGSSLRIRTLPWAHLKAAPFLTYFGLFQVH

SEQ ID NO: 14 - protein, 389 aa, Artificial Sequence
  Description of Artificial Sequence Synthetic polypeptide
  Human IFNbeta-LZ-TWEAK
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNSGGTSGSTSGTGSTVSSIEKKIEEITSQIIQISNEITLIRNEIAQIKQMKGRKTRARRAIAAHYEVHPRPGQDGAQAGVDGTVSGWEEARINSSSPLRYNRQIGEFIVTRAGLYYLYCQVHFDEGKAVYLKLDLLVDGVLALRCLEEFSATAASSLGPQLRLCQVSGLLALRPGSSLRIRTLPWAHLKAAPFLTYFGLFQVH

SEQ ID NO: 15 - protein, 609 aa, Artificial Sequence
  Description of Artificial Sequence Synthetic polypeptide
  Human IFNbeta-IgG1 FC-LIGHT
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNSGGTSGSTSGTGSDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGTSGSTSGTGSTDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 16 - protein, 368 aa, Artificial Sequence
  Description of Artificial Sequence Synthetic polypeptide
  Human IFNbeta-Linker-LIGHT
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNSGGTSGSTSGTGSTDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 17 - protein, 402 aa, Artificial Sequence
  Description of Artificial Sequence Synthetic polypeptide
  Human IFNbeta-LZ-LIGHT
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNSGGTSGSTSGTGSTVSSIEKKIEEITSQIIQISNEITLIRNEIAQIKQDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 18 - protein, 430 aa, Artificial Sequence
  Description of Artificial Sequence Synthetic polypeptide
  huIgG1-Fc-tweak
MTNKCLLQIALLLCFSTTALSSGGTSGSTSGTGSDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGTSGSTSGTGSTMKGRKTRARRAIAAHYEVHPRPGQDGAQAGVDGTVSGWEEARINSSSPLRYNRQIGEFIVTRAGLYYLYCQVHFDEGKAVYLKLDLLVDGVLALRCLEEFSATAASSLGPQLRLCQVSGLLALRPGSSLRIRTLPWAHLKAAPFLTYFGLFQVH

SEQ ID NO: 19 - protein, 441 aa, Artificial Sequence
  Description of Artificial Sequence Synthetic polypeptide
  huIgG1-Fc-Light
MTNKCLLQIALLLCFSTTALSSGGTSGSTSGTGSDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGTSGSTSGTGSTDGPAGSWEQLIQERRSHEVNPAAHGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 20 - protein, 419 aa, Artificial Sequence
  Description of Artificial Sequence Synthetic polypeptide
  mIFNb_Fc-mIgG1
MNNRWILHAAFLLCFSTTALSINYKQLQLQERTNIRKCQELLEQLNGKINLTYRADFKIPMEMTEKMQKSYTAFAIQEMLQNVFLVFRNNFSSTGWNETIVVRLLDELHQQTVFLKTVLEEKQEERLTWEMSSTALHLKSYYWRVQRYLKLMKYNSYAWMVVRAEIFRNFLIIRRLTRNFQNSGGTSGSTSGTGSVRSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 21 - protein, 422 aa, Artificial Sequence
  Description of Artificial Sequence Synthetic polypeptide
  Fc-mIgG1_mCD40L
MNNRWILHAAFLLCFSTTALSSGGTSGSTSGTGSVRSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKSGGTSGSTSGTGSTMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 22 - protein, 259 aa, Artificial Sequence
  Description of Artificial Sequence Synthetic polypeptide
  Fc-mIgG1
MNNRWILHAAFLLCFSTTALSSGGTSGSTSGTGSVRSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 23 - protein, 149 aa, Artificial Sequence
  Description of Artificial Sequence Synthetic polypeptide
  mCD40L soluble form
MQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 24 - protein, 182 aa, Artificial Sequence
  Description of Artificial Sequence Synthetic polypeptide
  mIFNb
MNNRWILHAAFLLCFSTTALSINYKQLQLQERTNIRKCQELLEQLNGKINLTYRADFKIPMEMTEKMQKSYTAFAIQEMLQNVFLVFRNNFSSTGWNETIVVRLLDELHQQTVFLKTVLEEKQEERLTWEMSSTALHLKSYYWRVQRYLKLMKYNSYAWMVVRAEIFRNFLIIRRLTRNFQN

SEQ ID NO: 25 - protein, 592 aa, Artificial Sequence
  Description of Artificial Sequence Synthetic polypeptide
  IFNa-G4S-huIgG1-LN2-huCD40L
MALTFALLVALLVLSCKSSCSVGCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSPAPDPAPDPSGMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 26 - protein, 215 aa, Artificial Sequence
  Description of Artificial Sequence Synthetic polypeptide
  Lz-Light
MSGGTSGSTSGTGSTVSSIEKKIEEITSQIIQISNEITLIRNEIAQIKQDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERVRLRDGTRSYFGAFMV

SEQ ID NO: 27 - protein, 15 aa, Artificial Sequence
  Description of Artificial Sequence Synthetic peptide
  Peptide Gly-Ser-Thr linker
GGGGSGGGGSGGGGS

SEQ ID NO: 28 - protein, 13 aa, Artificial Sequence
  Description of Artificial Sequence Synthetic peptide
  Peptide Gly-Ser-Thr linker
GSPAPDPAPDPSG

SEQ ID NO: 29 - protein, 21 aa, Artificial Sequence
  Description of Artificial Sequence Synthetic peptide
  Signal peptide 1
MTNKCLLQIALLLCFSTTALS

SEQ ID NO: 30 - protein, 21 aa, Artificial Sequence
  Description of Artificial Sequence Synthetic peptide
  Signal peptide 2
MNNRWILHAAFLLCFSTTALS

SEQ ID NO: 31 - protein, 18 aa, Artificial Sequence
  Description of Artificial Sequence Synthetic peptide
  Signal peptide 3
MALTFALLVALLVLSCKS

SEQ ID NO: 32 - protein, 15 aa, Artificial Sequence
  Description of Artificial Sequence Synthetic peptide
  Signal peptide 4
MSGGTSGSTSGTGST

SEQ ID NO: 33 - protein, 188 aa, Artificial Sequence [sequence omitted]

SEQ ID NO: 34 - DNA, 20 bp, Artificial Sequence
  Description of Artificial Sequence Synthetic oligonucleotide
  Forward primer for pgRNA
gccttagagtctcctgagca

SEQ ID NO: 35 - DNA, 20 bp, Artificial Sequence
  Description of Artificial Sequence Synthetic oligonucleotide
  Reverse primer for pgRNA
gagggagttcttcttctagg

SEQ ID NO: 36 - DNA, 24 bp, Artificial Sequence
  Description of Artificial Sequence Synthetic oligonucleotide
  Probe for pgRNA
agtgtggattcgcactcctccagc

(19) United States
(12) Patent Application Publication
(10) Pub. No.: US 2021/0260165 A1
(43) Pub. Date: Aug. 26, 2021

(54) COMBINATION THERAPY FOR TREATING HEPATITIS B VIRUS INFECTION

(71) Applicants: SANOFI, Paris (FR); EVOTEC INTERNATIONAL GmbH, Hamburg (DE)

(72) Inventors: Antoine ALAM, Lyon (FR); Odile BONNIN, Ancy (FR); Kara CARTER, Sudbury, MA (US); Julie MONTEGUT, Lyon (FR); Cendrine LEMOINE, Paris (FR); Xavier MARNIQUET, Lyon (FR)

(21) Appl. No.: 17/059,525

(22) PCT Filed: May 31, 2019
(86) PCT No.: PCT/EP2019/064239

§ 371 Date: Nov. 30, 2020

(30) Foreign Application Priority Data
Jun. 1, 2018 (EP) 18305675.3

(51) Int. Cl. A61K 38/21; A61K 38/17; A61P 31/20

(52) U.S. Cl. CPC A61K 38/215; A61P 31/20; A61K 38/212; A61K 38/177

(57) ABSTRACT

The invention relates to methods of treating a hepatitis B virus (HBV) infection in a subject in need thereof, comprising administering to the subject a combination of a tumor necrosis factor receptor superfamily (TNFRSF) agonist (e.g., a an agonistic antibody directed against the receptor, a soluble TNFRSF agonist including but not limited to its natural ligand or a fragment of either) and an interferon (IFN) or a functional fragment thereof, to decrease one or more symptoms of HBV infection in the subject are provided.

### FIELD OF THE INVENTION

The present invention relates to methods for treating hepatitis B virus (HBV) infection in a subject.

### BACKGROUND

HBV infects more than 300 million people worldwide and is a common cause of liver disease and liver cancer. (Liang (2009) Hepatology 49:S13.) HBV is a small DNA virus with unusual features similar to retroviruses, which replicates through an RNA intermediate (pregenomic RNA) and can integrate into the host genome. The unique features of the HBV replication cycle confer a distinct ability of the virus to persist in infected cells. HBV infection leads to a wide spectrum of liver disease ranging from acute (including fulminant hepatic failure) to chronic hepatitis, cirrhosis and hepatocellular carcinoma. Acute HBV infection can be either asymptomatic or present with symptomatic acute hepatitis. 90-95% of children and 5%-10% of adults infected with HBV are unable to clear the virus and become chronically infected. Many chronically infected persons have mild liver disease with little or no long-term morbidity or mortality. Other individuals with chronic HBV infection develop active disease, which can progress to cirrhosis and liver cancer. These patients require careful monitoring and warrant therapeutic intervention.

Novel methods for treating HBV infection by modulating HBV infection in a cell are needed. In particular, methods for effectively disrupting HBV viral load of HBV-infected cells, reducing transcription of covalently closed circular HBV DNA in HBV-infected cells, and/or reducing the amount of pre-genomic HBV RNA in HBV-infected cells are needed.

### SUMMARY OF THE INVENTION

The present invention is based on the discovery of novel methods for treating HBV infection in a subject using a combination of a tumor necrosis factor receptor superfamily (TNFRSF) agonist (e.g., an agonistic antibody directed against a member of the TNFRSF, a soluble TNFRSF agonist including but not limited to its natural ligand) or a functional fragment thereof, and an interferon (IFN) agent or a functional fragment thereof The novel methods described herein are useful for reducing transcription of covalently closed circular DNA (cccDNA) into pre-genomic RNA (pgRNA) in an HBV-infected cell, which in turn reduces HBV protein production by the HBV-infected cell, and ultimately reduces viral load of the HBV-infected cell.

In one aspect, a combination of a tumor necrosis factor receptor superfamily (TNFRSF) agonist or a functional fragment thereof and an interferon (IFN) agent or a functional fragment thereof, for use for treating a HBV infection is provided.

In a particular embodiment, the TNFRSF agonist or a functional fragment thereof is selected from the group consisting of a lymphotoxin alpha 3 receptor agonist, a lymphotoxin beta receptor agonist, a herpesvirus entry mediator agonist, a tumor necrosis factor-like receptor weak inducer of apoptosis agonist, a cluster of differentiation factor 40 agonist, a CD27 agonist, a CD30 agonist, a 4-1BB agonist, a receptor activator of nuclear factor κB agonist, a Troy agonist, and a OX40 receptor agonist, or functional fragments thereof.

In a particular embodiment, the TNFRSF agonist or a functional fragment thereof is selected from the group consisting of CD40L, LTα3, LIGHT and TWEAK, or functional fragments thereof. In a particular embodiment, the TNFRSF agonist or a functional fragment thereof is selected from the group consisting of CD40L, LIGHT and TWEAK, or functional fragments thereof.

In a particular embodiment, the TNFRSF agonist or a functional fragment thereof is a CD40 agonist or a functional fragment thereof selected from the group consisting of a CD40 ligand (CD40L) or a functional fragment thereof, an agonistic anti-CD40 antibody, a functional fragment thereof or antigen-binding fragment thereof, and a fusion protein comprising a CD40 ligand or a functional fragment thereof. In a particular embodiment, the CD40L is hexameric CD40L or trimeric CD40L.

In a particular embodiment, the TNFRSF agonist or a functional fragment thereof is a polypeptide or a functional fragment thereof, an antibody or a functional fragment thereof, or an antigen-binding fragment thereof.

In a particular embodiment, the TNFRSF agonist or a functional fragment thereof is provided as a fusion protein comprising said TNFRSF agonist or a functional fragment thereof.

In a particular embodiment, the IFN agent or a functional fragment thereof is selected from the group consisting of a Type I IFN agent, a Type II IFN agent and a Type III IFN agent, or functional fragments thereof.

In a particular embodiment, the IFN agent or a functional fragment thereof is IFNα, IFNβ, IFNγ or IFNλ, or functional fragments thereof. In a particular embodiment, the IFN agent or a functional fragment thereof is IFNβ or IFNγ or functional fragments thereof. In a particular embodiment, the IFN agent or a functional fragment thereof is IFNβ, or a functional fragment thereof. In a particular embodiment, the IFN agent or a functional fragment thereof is IFNα, or a functional fragment thereof.

In a particular embodiment, the IFN agent or a functional fragment thereof is provided as a fusion protein comprising said IFN agent or a functional fragment thereof.

In a particular embodiment, the TNFRSF agonist or a functional fragment thereof and the IFN agent or a functional fragment thereof are provided as a bifunctional immunostimulatory fusion protein comprising the said TNFRSF agonist or a functional fragment thereof, the said IFN agent or a functional fragment thereof and a linker.

In a particular embodiment, the TNFRSF agonist or a functional fragment thereof and the IFN agent or a functional fragment thereof are comprised in a single pharmaceutical composition.

In a particular embodiment, the TNFRSF agonist or a functional fragment thereof and the IFN agent or a functional fragment thereof are comprised in distinct pharmaceutical compositions.

In another aspect, a pharmaceutical composition comprising: a TNFRSF agonist or a functional fragment thereof; and a Type II IFN agent, a functional fragment of a Type II IFN agent, a Type III IFN agent, or a functional fragment of a Type III IFN agent.

In another aspect, a method of treating an HBV infection and/or one or more HBV-related symptoms in a subject in need thereof, comprising administering to the subject a combination of a TNFRSF agonist (e.g., an agonistic antibody directed against a member of the TNFRSF, a soluble TNFRSF agonist including but not limited to its natural ligand) or a functional fragment thereof, and an IFN agent or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In another aspect, a method of treating HBV infection in a subject in need thereof, comprising administering to the subject a combination of:

(i) a TNFRSF agonist selected from the group consisting of a lymphotoxin alpha 3 (LTα3) receptor (TNFRSF1A, TNFRSF1B, or TNFRSF14) agonist (e.g. LTα3), a lymphotoxin beta (LTβ) receptor (TNFRSF3) agonist (e.g., LIGHT or LTβ), a herpesvirus entry mediator (HVEM or TNFRSF14) agonist (e.g. LIGHT), a tumor necrosis factor-like receptor weak inducer of apoptosis (TNFRSF12A) agonist (e.g., TWEAK also known as TNFSF12), a cluster of differentiation factor 40 (CD40, TNFRSF5) agonist (e.g. CD40L), a CD27 (TNFRSF7) agonist (e.g. CD70), a CD30 (TNFRSF8) agonist, a 4-1BB (CD137, TNFRSF9) agonist, a receptor activator of nuclear factor κB (RANK, TNFRSF11A) agonist, a Troy (TNFRSF19) agonist, and a OX40 receptor (TNFRSF4) agonist or a functional fragment thereof; and

(ii) an IFN agent or a functional fragment thereof,

to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In another aspect, a method of treating HBV infection in a subject in need thereof, comprising administering to the subject a combination of:

(i) a TNFRSF agonist selected from the group consisting of a lymphotoxin alpha 3 (LTα3) receptor (TNFRSF1A, TNFRSF1B, or TNFRSF14) agonist (e.g. LTα3), a lymphotoxin beta (LTβ) receptor (TNFRSF3) agonist (e.g., LIGHT or LTβ), a herpesvirus entry mediator (HVEM or TNFRSF14) agonist (LIGHT), a tumor necrosis factor-like receptor weak inducer of apoptosis (TNFRSF12A) agonist (e.g., TWEAK also known as TNFSF12), a cluster of differentiation factor 40 (CD40, TNFRSF5) agonist (CD40L), a CD27 (TNFRSF7) agonist (CD70), a CD30 (TNFRSF8) agonist, a 4-1BB (CD137, TNFRSF9) agonist, a receptor activator of nuclear factor κB (RANK, TNFRSF11A) agonist, a Troy (TNFRSF19) agonist, and a OX40 receptor (TNFRSF4) agonist or a functional fragment thereof; and

(ii) an IFN agent or a functional fragment thereof,

to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In another aspect, a method of treating HBV infection in a subject in need thereof, comprising administering to the subject a combination of:

(i) a TNFRSF agonist selected from the group consisting of a lymphotoxin alpha 3 (LTα3) receptor (TNFRSF1A, TNFRSF1B, or TNFRSF14) agonist (e.g. LTα3), a lymphotoxin beta (LTβ) receptor (TNFRSF3) agonist (e.g., LIGHT or LTβ), a herpesvirus entry mediator (HVEM or TNFRSF14) agonist (e.g. LIGHT), a tumor necrosis factor-like receptor weak inducer of apoptosis (TNFRSF12A) agonist (e.g., TWEAK also known as TNFSF12), a cluster of differentiation factor 40 (CD40, TNFRSF5) agonist (e.g. CD40L), a CD27 (TNFRSF7) agonist (e.g. CD70), a CD30 (TNFRSF8) agonist, a receptor activator of nuclear factor κB (RANK, TNFRSF11A) agonist, a Troy (TNFRSF19) agonist, and a OX40 receptor (TNFRSF4) agonist or a functional fragment thereof; and

(ii) an IFN agent or a functional fragment thereof,

to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

According to a particular embodiment, a method of treating HBV infection in a subject in need thereof, comprising administering to the subject a combination of:

(i) a TNFRSF agonist selected from the group consisting of a lymphotoxin alpha 3 (LTα3) receptor (TNFRSF1A, TNFRSF1B, or TNFRSF14) agonist (e.g. LTα3), a lymphotoxin beta (LTβ) receptor (TNFRSF3) agonist (e.g., LIGHT or LTβ), a herpesvirus entry mediator (HVEM or TNFRSF14) agonist (LIGHT), a tumor necrosis factor-like receptor weak inducer of apoptosis (TNFRSF12A) agonist (e.g., TWEAK also known as TNFSF12), a cluster of differentiation factor 40 (CD40, TNFRSF5) agonist (CD40L), a CD27 (TNFRSF7) agonist (CD70), a CD30 (TNFRSF8) agonist, a receptor activator of nuclear factor κB (RANK, TNFRSF11A) agonist, a Troy (TNFRSF19) agonist, and a OX40 receptor (TNFRSF4) agonist or a functional fragment thereof; and

(ii) an IFN agent or a functional fragment thereof,

to decrease one or more symptoms of HBV infection in the subject, is provided. According to the said embodiment, the said combination for use in the treatment of an HBV infection, and/or for decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In another aspect, a method of treating HBV infection in a subject in need thereof, comprising administering to the subject a combination of a TNFRSF agonist or a functional fragment thereof; and a Type I IFN, or a functional fragment of a Type I IFN, or a Type II IFN, or a functional fragment of a Type II IFN, or a Type III IFN, or a functional fragment of a Type III IFN, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In another aspect, a method of treating HBV infection in a subject in need thereof, comprising administering to the subject a combination of a TNFRSF agonist or a functional fragment thereof; and a Type I IFN, or a functional fragment of a Type I IFN, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In another aspect, a method of treating HBV infection in a subject in need thereof, comprising administering to the subject a combination of a TNFRSF agonist or a functional fragment thereof; and a Type II IFN, or a functional fragment of a Type II IFN, or a Type III IFN, or a functional fragment of a Type III IFN, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In another aspect, a method of treating HBV infection in a subject in need thereof, comprising administering to the subject a combination of a TNFRSF agonist selected from the group consisting of LTα3 receptor agonist, tumor necrosis factor-like receptor weak inducer of apoptosis (TNFRSF12A) agonist, LTβ receptor (TNFRSF3) agonist, herpesvirus entry mediator (HVEM or TNFRSF14) agonist and cluster of differentiation factor 40 (CD40, TNFRSF5) agonist or a functional fragment thereof; and an IFN agent or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating an HBV infection, and/or for decreasing one or more symptoms of HBV infection in the subject, is provided.

In a particular embodiment, a method of treating HBV infection in a subject in need thereof, comprising administering to the subject a combination of a TNFRSF agonist selected from the group consisting of LTα3 receptor agonist, tumor necrosis factor-like receptor weak inducer of apoptosis (TNFRSF12A) agonist, LTβ receptor (TNFRSF5) agonist, and cluster of differentiation factor 40 (CD40, TNFRSF5) agonist or a functional fragment thereof and an IFN agent or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating an HBV infection, and/or for decreasing one or more symptoms of HBV infection in the subject, is provided.

In a particular embodiment, a method of treating HBV infection in a subject in need thereof, comprising administering to the subject a combination of a TNFRSF agonist selected from the group consisting of LTα3 receptor agonist, tumor necrosis factor-like receptor weak inducer of apoptosis (TNFRSF12A) agonist, herpesvirus entry mediator (HVEM or TNFRSF14) agonist and cluster of differentiation factor 40 (CD40, TNFRSF5) agonist or a functional fragment thereof and an IFN agent or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating an HBV infection, and/or for decreasing one or more symptoms of HBV infection in the subject, is provided

In a particular embodiment, a method of treating HBV infection in a subject in need thereof, comprising administering to the subject a combination of a TNFRSF agonist selected from the group consisting of LTα3, TWEAK, LIGHT, and CD40L or a functional fragment thereof and an IFN agent or a functional fragment thereof, to decrease one or more symptoms of HBV infection in the subject, is provided. According to the said embodiment, the said combination for use in the treatment of an HBV infection, and/or for decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In another aspect, a method of treating HBV infection in a subject in need thereof, comprising administering to the subject a combination of a TNFRSF agonist selected from the group consisting of LTα3 receptor agonist, tumor necrosis factor-like receptor weak inducer of apoptosis (TNFRSF12A) agonist, LTβ receptor (TNFRSF3) agonist and herpes virus entry mediator (HVEM or TNFRSF14) agonist or a functional fragment thereof and an IFN agent or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In a particular embodiment, method of treating HBV infection in a subject in need thereof, comprising administering to the subject a combination of a TNFRSF agonist selected from the group consisting of LTα3 receptor agonist, tumor necrosis factor-like receptor weak inducer of apoptosis (TNFRSF12A) agonist, and LTβ receptor (TNFRSF3) agonist or a functional fragment thereof and an IFN agent or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In a particular embodiment, method of treating HBV infection in a subject in need thereof, comprising administering to the subject a combination of a TNFRSF agonist selected from the group consisting of LTα3 receptor agonist, tumor necrosis factor-like receptor weak inducer of apoptosis (TNFRSF12A) agonist, and herpes virus entry mediator (HVEM or TNFRSF14) agonist or a functional fragment thereof; and an IFN agent or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In a particular embodiment, a method of treating HBV infection in a subject in need thereof, comprising administering to the subject a combination of a TNFRSF agonist selected from the group consisting of a LTα3 receptor agonist, LTβ receptor (TNFRSF3) agonist, a herpes virus entry mediator (HVEM or TNFRSF14) agonist and a tumor necrosis factor-like receptor weak inducer of apoptosis (TNFRSF12A) agonist, or a functional fragment thereof; and an IFN agent or a functional fragment thereof, to decrease one or more symptoms of HBV infection in the subject, is provided. According to the said embodiment, the said combination for use in the treatment of an HBV infection, and/or for decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In a particular embodiment, a method of treating HBV infection in a subject in need thereof, comprising administering to the subject a combination of a TNFRSF agonist selected from the group consisting of a LTα3 receptor agonist, LTβ receptor (TNFRSF3) agonist, and a tumor necrosis factor-like receptor weak inducer of apoptosis (TNFRSF12A) agonist, or a functional fragment thereof; and an IFN agent or a functional fragment thereof, to decrease one or more symptoms of HBV infection in the subject, is provided. According to the said embodiment, the said combination for use in the treatment of an HBV infection, and/or for decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In a particular embodiment, a method of treating HBV infection in a subject in need thereof, comprising administering to the subject a combination of a TNFRSF agonist selected from the group consisting of a LTα3 receptor agonist, a herpes virus entry mediator (HVEM or TNFRSF14) agonist and a tumor necrosis factor-like receptor weak inducer of apoptosis (TNFRSF12A) agonist, or a functional fragment thereof; and an IFN agent or a functional fragment thereof, to decrease one or more symptoms of HBV infection in the subject, is provided. According to the said embodiment, the said combination for use in the treatment of an HBV infection, and/or for decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In a particular embodiment, a method of treating HBV infection in a subject in need thereof, comprising administering to the subject a combination of a TNFRSF agonist selected from the group consisting of LTα3, TWEAK, and LIGHT or a functional fragment thereof; and an IFN agent or a functional fragment thereof, to decrease one or more symptoms of HBV infection in the subject, is provided. According to the said embodiment, the said combination for use in the treatment of an HBV infection, and/or for decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In another aspect, a method of treating HBV infection in a subject in need thereof, comprising administering to the subject a combination of a TNFRSF agonist selected from the group consisting of LTβ receptor (TNFRSF3) agonist, a herpes virus entry mediator (HVEM or TNFRSF14) agonist, a tumor necrosis factor-like receptor weak inducer of apoptosis (TNFRSF12A) agonist and a cluster of differentiation factor 40 (CD40, TNFRSF5) agonist or a functional fragment thereof; and an IFN agent or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In a particular embodiment, a method of treating HBV infection in a subject in need thereof, comprising administering to the subject a combination of a TNFRSF agonist selected from the group consisting of LTβ receptor (TNFRSF3) agonist, a tumor necrosis factor-like receptor weak inducer of apoptosis (TNFRSF12A) agonist and a cluster of differentiation factor 40 (CD40, TNFRSF5) agonist or a functional fragment thereof; and an IFN agent or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In a particular embodiment, a method of treating HBV infection in a subject in need thereof, comprising administering to the subject a combination of a TNFRSF agonist selected from the group consisting of a herpes virus entry mediator (HVEM or TNFRSF14) agonist, a tumor necrosis factor-like receptor weak inducer of apoptosis (TNFRSF12A) agonist and a cluster of differentiation factor 40 (CD40, TNFRSF5) agonist or a functional fragment thereof; and an IFN agent or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In a particular embodiment, a method of treating HBV infection in a subject in need thereof, comprising administering to the subject a combination of a TNFRSF agonist selected from the group consisting of CD40L, TWEAK, and LIGHT or a functional fragment thereof; and an IFN agent or a functional fragment thereof, to decrease one or more symptoms of HBV infection in the subject, is provided. According to the said embodiment, the said combination for use in the treatment of an HBV infection, and/or for decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In another aspect, a method of treating HBV infection in a subject in need thereof, comprising administering to the subject a combination of a cluster of differentiation factor 40 (CD40, TNFRSF5) agonist or a functional fragment thereof, and an IFN agent or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating an HBV infection, and/or for decreasing one or more symptoms of HBV infection in the subject, is provided.

In a particular embodiment, a method of treating HBV infection in a subject in need thereof, comprising administering to the subject a combination of CD40L or a functional fragment thereof, and an IFN agent or a functional fragment thereof, to decrease one or more symptoms of HBV infection in the subject, is provided. According to the said embodiment, the said combination for use in the treatment of an HBV infection, and/or for decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In another aspect, a method of treating HBV infection in a subject in need thereof, comprising administering to the subject a combination of a tumor necrosis factor-like receptor weak inducer of apoptosis (TNFRSF12A) agonist, and in particular TWEAK, or a functional fragment thereof, and an IFN agent or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In another aspect, a method of treating HBV infection in a subject in need thereof, comprising administering to the subject a combination of a LTβ receptor (TNFRSF3) agonist or herpesvirus entry mediator (HVEM or TNFRSF14) agonist, and in particular LIGHT, or a functional fragment thereof, and an IFN agent or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating an HBV infection, and/or for decreasing one or more symptoms of HBV infection in the subject, is provided.

In a particular embodiment, a method of treating HBV infection in a subject in need thereof, comprising administering to the subject a combination of a LTβ receptor (TNFRSF3) agonist and in particular LIGHT, or a functional fragment thereof, and an IFN agent or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating an HBV infection, and/or for decreasing one or more symptoms of HBV infection in the subject, is provided.

In a particular embodiment, a method of treating HBV infection in a subject in need thereof, comprising administering to the subject a combination of a herpesvirus entry mediator (HVEM or TNFRSF14) agonist, and in particular LIGHT, or a functional fragment thereof, and an IFN agent or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating an HBV infection, and/or for decreasing one or more symptoms of HBV infection in the subject, is provided.

In another aspect, a method of treating HBV infection in a subject in need thereof, comprising administering to the subject a combination of a TNFRSF agonist or a functional fragment thereof; and an IFN agent selected from the group consisting of IFNα, IFNβ, IFNγ and IFNλ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In another aspect, a method of treating HBV infection in a subject in need thereof, comprising administering to the subject a combination of a TNFRSF agonist or a functional fragment thereof; and an IFN agent selected from the group consisting of IFNα and IFNβ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In another aspect, a method of treating HBV infection in a subject in need thereof, comprising administering to the subject a combination of a TNFRSF agonist or a functional fragment thereof; and an IFN agent selected from the group consisting of IFNγ and IFNλ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In another aspect, a method of treating HBV infection and HBV-related symptoms in a subject in need thereof, comprising administering to the subject a combination of a TNFRSF agonist selected from the group consisting of LTα3 receptor agonist, a LTβ receptor (TNFRSF3) agonist, a herpes virus entry mediator (HVEM or TNFRSF14) agonist, a tumor necrosis factor-like receptor weak inducer of apoptosis (TNFRSF12A) agonist and a cluster of differentiation factor 40 (CD40, TNFRSF5) agonist, or a functional fragment thereof; and an IFN agent selected from the group consisting of IFNα, IFNβ, IFNγ and IFNλ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In a particular embodiment, a method of treating HBV infection and HBV-related symptoms in a subject in need thereof, comprising administering to the subject a combination of a TNFRSF agonist selected from the group consisting of LTα3 receptor agonist, a LTβ receptor (TNFRSF3) agonist, a tumor necrosis factor-like receptor weak inducer of apoptosis (TNFRSF12A) agonist and a cluster of differentiation factor 40 (CD40, TNFRSF5) agonist, or a functional fragment thereof; and an IFN agent selected from the group consisting of IFNα, IFNβ, IFNγ and IFNλ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In a particular embodiment, a method of treating HBV infection and HBV-related symptoms in a subject in need thereof, comprising administering to the subject a combination of a TNFRSF agonist selected from the group consisting of LTα3 receptor agonist, a herpes virus entry mediator (HVEM or TNFRSF14) agonist, a tumor necrosis factor-like receptor weak inducer of apoptosis (TNFRSF12A) agonist and a cluster of differentiation factor 40 (CD40, TNFRSF5) agonist, or a functional fragment thereof; and an IFN agent selected from the group consisting of IFNα, IFNβ, IFNγ and IFNλ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

According to a particular embodiment, a method of treating HBV infection and HBV-related symptoms in a subject in need thereof, comprising administering to the subject a combination of a TNFRSF agonist selected from the group consisting of LTα3, TWEAK, LIGHT, and CD40L or a functional fragment thereof; and an IFN agent selected from the group consisting of IFNα, IFNβ, IFNγ and IFNλ or a functional fragment thereof, to decrease one or more symptoms of HBV infection in the subject, is provided. According to the said embodiment, the said combination for use in the treatment of an HBV infection, and/or for decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In another aspect, a method of treating HBV infection and HBV-related symptoms in a subject in need thereof, comprising administering to the subject a combination of a TNFRSF agonist selected from the group consisting of a LTα3 receptor agonist, a LTβ receptor (TNFRSF3) agonist, a herpes virus entry mediator (HVEM or TNFRSF14) agonist and a tumor necrosis factor-like receptor weak inducer of apoptosis (TNFRSF12A) agonist or a functional fragment thereof and an IFN agent selected from the group consisting of IFNα and IFNβ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating an HBV infection, and/or for decreasing one or more symptoms of HBV infection in the subject, is provided.

In a particular embodiment, a method of treating HBV infection and HBV-related symptoms in a subject in need thereof, comprising administering to the subject a combination of a TNFRSF agonist selected from the group consisting of a LTα3 receptor agonist, a LTβ receptor (TNFRSF3) agonist, and a tumor necrosis factor-like receptor weak inducer of apoptosis (TNFRSF12A) agonist or a functional fragment thereof and an IFN agent selected from the group consisting of IFNα and IFNβ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating an HBV infection, and/or for decreasing one or more symptoms of HBV infection in the subject, is provided.

In a particular embodiment, a method of treating HBV infection and HBV-related symptoms in a subject in need thereof, comprising administering to the subject a combination of a TNFRSF agonist selected from the group consisting of a LTα3 receptor agonist, a herpes virus entry mediator (HVEM or TNFRSF14) agonist and a tumor necrosis factor-like receptor weak inducer of apoptosis (TNFRSF12A) agonist or a functional fragment thereof and an IFN agent selected from the group consisting of IFNα and IFNβ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating an HBV infection, and/or for decreasing one or more symptoms of HBV infection in the subject, is provided.

In a particular embodiment, a method of treating HBV infection and HBV-related symptoms in a subject in need thereof, comprising administering to the subject a combination of a TNFRSF agonist selected from the group consisting of LTα3, TWEAK, and LIGHT or a functional fragment thereof and an IFN agent selected from the group consisting of IFNα and IFNβ or a functional fragment thereof, to decrease one or more symptoms of HBV infection in the subject, is provided. According to the said embodiment, the said combination for use in the treatment of an HBV infection, and/or for decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In another aspect, a method of treating HBV infection and HBV-related symptoms in a subject in need thereof, comprising administering to the subject a combination of a TNFRSF agonist selected from the group consisting of a LTβ receptor (TNFRSF3) agonist, a herpes virus entry mediator (HVEM or TNFRSF14) agonist, a tumor necrosis factor-like receptor weak inducer of apoptosis (TNFRSF12A) agonist and a cluster of differentiation factor 40 (CD40, TNFRSF5) agonist or a functional fragment thereof; and an IFN agent selected from the group consisting of IFNα and IFNβ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating an HBV infection, and/or for decreasing one or more symptoms of HBV infection in the subject, is provided.

In a particular embodiment, a method of treating HBV infection and HBV-related symptoms in a subject in need thereof, comprising administering to the subject a combination of a TNFRSF agonist selected from the group consisting of a LTβ receptor (TNFRSF3) agonist, a tumor necrosis factor-like receptor weak inducer of apoptosis (TNFRSF12A) agonist and a cluster of differentiation factor 40 (CD40, TNFRSF5) agonist or a functional fragment thereof; and an IFN agent selected from the group consisting of IFNα and IFNβ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating an HBV infection, and/or for decreasing one or more symptoms of HBV infection in the subject, is provided.

In a particular embodiment, a method of treating HBV infection and HBV-related symptoms in a subject in need thereof, comprising administering to the subject a combination of a TNFRSF agonist selected from the group consisting of a herpes virus entry mediator (HVEM or TNFRSF14) agonist, a tumor necrosis factor-like receptor weak inducer of apoptosis (TNFRSF12A) agonist and a cluster of differentiation factor 40 (CD40, TNFRSF5) agonist or a functional fragment thereof; and an IFN agent selected from the group consisting of IFNα and IFNβ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating an HBV infection, and/or for decreasing one or more symptoms of HBV infection in the subject, is provided.

In a particular embodiment, a method of treating HBV infection and HBV-related symptoms in a subject in need thereof, comprising administering to the subject a combination of a TNFRSF agonist selected from the group consisting of TWEAK, LIGHT and CD40L or a functional fragment thereof; and an IFN agent selected from the group consisting of IFNα and IFNβ or a functional fragment thereof, to decrease one or more symptoms of HBV infection in the subject, is provided. According to the said embodiment, the said combination for use in the treatment of an HBV infection, and/or for decreasing one or more symptoms of HBV infection in the subject, is provided. Still according to the said embodiment, the use of the said combination for preparing a medicament for treating an HBV infection, and/or for decreasing one or more symptoms of HBV infection in the subject, is provided.

In another aspect, a method of treating HBV infection and HBV-related symptoms in a subject in need thereof, comprising administering to the subject a combination of a TNFRSF agonist selected from the group consisting of a LTα3 receptor agonist, a LTβ receptor (TNFRSF3) agonist, a herpes virus entry mediator (HVEM or TNFRSF14) agonist and a tumor necrosis factor-like receptor weak inducer of apoptosis (TNFRSF12A) agonist or a functional fragment thereof; and an IFN agent selected from the group consisting of IFNγ and IFNλ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In a particular embodiment, a method of treating HBV infection and HBV-related symptoms in a subject in need thereof, comprising administering to the subject a combination of a TNFRSF agonist selected from the group consisting of a LTα3 receptor agonist, a LTβ receptor (TNFRSF3) agonist, and a tumor necrosis factor-like receptor weak inducer of apoptosis (TNFRSF12A) agonist or a functional fragment thereof; and an IFN agent selected from the group consisting of IFNγ and IFNλ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In a particular embodiment, a method of treating HBV infection and HBV-related symptoms in a subject in need thereof, comprising administering to the subject a combination of a TNFRSF agonist selected from the group consisting of a LTα3 receptor agonist, a herpes virus entry mediator (HVEM or TNFRSF14) agonist and a tumor necrosis factor-like receptor weak inducer of apoptosis (TNFRSF12A) agonist or a functional fragment thereof; and an IFN agent selected from the group consisting of IFNγ and IFNλ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

According to a particular embodiment, a method of treating HBV infection and HBV-related symptoms in a subject in need thereof, comprising administering to the subject a combination of a TNFRSF agonist selected from the group consisting of LTα3, TWEAK, and LIGHT or a functional fragment thereof; and an IFN agent selected from the group consisting of IFNγ and IFNλ or a functional fragment thereof, to decrease one or more symptoms of HBV infection in the subject, is provided. According to the said embodiment, the said combination for use in the treatment of an HBV infection, and/or for decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In another aspect, a method of treating HBV infection and HBV-related symptoms in a subject in need thereof, comprising administering to the subject a combination of a TNFRSF agonist selected from the group consisting of a LTβ receptor (TNFRSF3) agonist, a herpes virus entry mediator (HVEM or TNFRSF14) agonist, a tumor necrosis factor-like receptor weak inducer of apoptosis (TNFRSF12A) agonist and a cluster of differentiation factor 40 (CD40, TNFRSF5) agonist or a functional fragment thereof; and an IFN agent selected from the group consisting of IFNγ and IFNλ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In a particular embodiment, a method of treating HBV infection and HBV-related symptoms in a subject in need thereof, comprising administering to the subject a combination of a TNFRSF agonist selected from the group consisting of a LTβ receptor (TNFRSF3) agonist, a tumor necrosis factor-like receptor weak inducer of apoptosis (TNFRSF12A) agonist and a cluster of differentiation factor 40 (CD40, TNFRSF5) agonist or a functional fragment thereof; and an IFN agent selected from the group consisting of IFNγ and IFNλ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In a particular embodiment, a method of treating HBV infection and HBV-related symptoms in a subject in need thereof, comprising administering to the subject a combination of a TNFRSF agonist selected from the group consisting of a herpes virus entry mediator (HVEM or TNFRSF14) agonist, a tumor necrosis factor-like receptor weak inducer of apoptosis (TNFRSF12A) agonist and a cluster of differentiation factor 40 (CD40, TNFRSF5) agonist or a functional fragment thereof and an IFN agent selected from the group consisting of IFNγ and IFNλ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In a particular embodiment, a method of treating HBV infection and HBV-related symptoms in a subject in need thereof, comprising administering to the subject a combination of a TNFRSF agonist selected from the group consisting of TWEAK, LIGHT and CD40L or a functional fragment thereof and an IFN agent selected from the group consisting of IFNγ and IFNλ or a functional fragment thereof, to decrease one or more symptoms of HBV infection in the subject, is provided. According to the said embodiment, the said combination for use in the treatment of an HBV infection, and/or for decreasing one or more symptoms of HBV infection in the subject, is provided. Still according to the said embodiment, the use of the said combination for preparing a medicament for treating an HBV infection, and/or for decreasing one or more symptoms of HBV infection in the subject, is provided.

In another aspect, a method of treating HBV infection and HBV-related symptoms in a subject in need thereof, comprising administering to the subject a combination of a cluster of differentiation factor 40 (CD40, TNFRSF5) agonist or a functional fragment thereof; and an IFN agent selected from the group consisting of IFNα and IFNβ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating an HBV infection, and/or for decreasing one or more symptoms of HBV infection in the subject, is provided.

According to a particular embodiment, a method of treating HBV infection and HBV-related symptoms in a subject in need thereof, comprising administering to the subject a combination of CD40L or a functional fragment thereof; and an IFN agent selected from the group consisting of IFNα and IFNβ or a functional fragment thereof, to decrease one or more symptoms of HBV infection in the subject, is provided. According to the said embodiment, the said combination for use in the treatment of an HBV infection, and/or for decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In another aspect, a method of treating HBV infection and HBV-related symptoms in a subject in need thereof, comprising administering to the subject a combination of a tumor necrosis factor-like receptor weak inducer of apoptosis (TNFRSF12A) agonist, and in particular TWEAK, or a functional fragment thereof; and an IFN agent selected from the group consisting of IFNα and IFNβ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In another aspect, a method of treating HBV infection and HBV-related symptoms in a subject in need thereof, comprising administering to the subject a combination of a LTβ receptor (TNFRSF3) agonist, a herpesvirus entry mediator (HVEM or TNFRSF14) agonist, and in particular LIGHT, or a functional fragment thereof; and an IFN agent selected from the group consisting of IFNα and IFNβ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In a particular embodiment, a method of treating HBV infection and HBV-related symptoms in a subject in need thereof, comprising administering to the subject a combination of a LTβ receptor (TNFRSF3) agonist, and in particular LIGHT, or a functional fragment thereof; and an IFN agent selected from the group consisting of IFNα and IFNβ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In a particular embodiment, a method of treating HBV infection and HBV-related symptoms in a subject in need thereof, comprising administering to the subject a combination of a herpesvirus entry mediator (HVEM or TNFRSF14) agonist, and in particular LIGHT, or a functional fragment thereof; and an IFN agent selected from the group consisting of IFNα and IFNβ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In another aspect, a method of treating HBV infection and HBV-related symptoms in a subject in need thereof, comprising administering to the subject a cluster of differentiation factor 40 (CD40, TNFRSF5) agonist or a functional fragment thereof; and an IFNλ agent selected from the group consisting of IL-28 and IL-29 or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating an HBV infection, and/or for decreasing one or more symptoms of HBV infection in the subject, is provided.

In a particular embodiment, a method of treating HBV infection and HBV-related symptoms in a subject in need thereof, comprising administering to the subject CD40L or a functional fragment thereof; and an IFNλ agent selected from the group consisting of IL-28 and IL-29 or a functional fragment thereof, to decrease one or more symptoms of HBV infection in the subject, is provided. According to the said embodiment, the said combination for use in the treatment of an HBV infection, and/or for decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In another aspect, a method of treating HBV infection and HBV-related symptoms in a subject in need thereof, comprising administering to the subject a tumor necrosis factor-like receptor weak inducer of apoptosis (TNFRSF12A) agonist, and in particular TWEAK, or a functional fragment thereof; and an IFNλ, agent selected from the group consisting of IL-28 and IL-29 or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating an HBV infection, and/or for decreasing one or more symptoms of HBV infection in the subject, is provided.

In another aspect, a method of treating HBV infection and HBV-related symptoms in a subject in need thereof, comprising administering to the subject a LTβ receptor (TNFRSF3) agonist, a herpesvirus entry mediator (HVEM or TNFRSF14) agonist, and in particular LIGHT, or a functional fragment thereof and an IFNλ agent selected from the group consisting of IL-28 and IL-29 or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating an HBV infection, and/or for decreasing one or more symptoms of HBV infection in the subject, is provided.

In a particular embodiment, a method of treating HBV infection and HBV-related symptoms in a subject in need thereof, comprising administering to the subject a LTβ receptor (TNFRSF3) agonist, and in particular LIGHT, or a functional fragment thereof and an IFNλ agent selected from the group consisting of IL-28 and IL-29 or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating an HBV infection, and/or for decreasing one or more symptoms of HBV infection in the subject, is provided.

In a particular embodiment, a method of treating HBV infection and HBV-related symptoms in a subject in need thereof, comprising administering to the subject a herpesvirus entry mediator (HVEM or TNFRSF14) agonist, and in particular LIGHT, or a functional fragment thereof; and an IFNλ agent selected from the group consisting of IL-28 and IL-29 or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating an HBV infection, and/or for decreasing one or more symptoms of HBV infection in the subject, is provided.

In another aspect, a method of treating HBV infection and HBV-related symptoms in a subject in need thereof, comprising administering to the subject a combination of a cluster of differentiation factor 40 (CD40, TNFRSF5) agonist or a functional fragment thereof; and IFNγ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating an HBV infection, and/or for decreasing one or more symptoms of HBV infection in the subject, is provided.

According to a particular embodiment, a method of treating HBV infection and HBV-related symptoms in a subject in need thereof, comprising administering to the subject a combination of CD40L or a functional fragment thereof; and IFNγ or a functional fragment thereof, to decrease one or more symptoms of HBV infection in the subject, is provided. According to the said embodiment, the said combination for use in the treatment of an HBV infection, and/or for decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In another aspect, a method of treating HBV infection and HBV-related symptoms in a subject in need thereof, comprising administering to the subject a combination of a tumor necrosis factor-like receptor weak inducer of apoptosis (TNFRSF12A) agonist, and in particular TWEAK, or a functional fragment thereof and IFNγ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating an HBV infection, and/or for decreasing one or more symptoms of HBV infection in the subject, is provided.

In another aspect, a method of treating HBV infection and HBV-related symptoms in a subject in need thereof, comprising administering to the subject a combination of a LTβ receptor (TNFRSF3) agonist, a herpesvirus entry mediator (HVEM or TNFRSF14) agonist, and in particular LIGHT, or a functional fragment thereof and IFNγ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In a particular embodiment, a method of treating HBV infection and HBV-related symptoms in a subject in need thereof, comprising administering to the subject a combination of a LTβ receptor (TNFRSF3) agonist, and in particular LIGHT, or a functional fragment thereof and IFNγ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In a particular embodiment, a method of treating HBV infection and HBV-related symptoms in a subject in need thereof, comprising administering to the subject a combination of a herpesvirus entry mediator (HVEM or TNFRSF14) agonist, and in particular LIGHT, or a functional fragment thereof and IFNγ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In another aspect, a method of treating HBV infection and HBV-related symptoms in a subject in need thereof, comprising administering to the subject a combination of a LTα3 receptor agonist or a functional fragment thereof and an IFN agent selected from the group consisting of IFNα and IFNβ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating an HBV infection, and/or for decreasing one or more symptoms of HBV infection in the subject, is provided.

In a particular embodiment, a method of treating HBV infection and HBV-related symptoms in a subject in need thereof, comprising administering to the subject a combination of LTα3 or a functional fragment thereof and an IFN agent selected from the group consisting of IFNα and IFNβ or a functional fragment thereof, to decrease one or more symptoms of HBV infection in the subject, is provided. According to the said embodiment, the said combination for use in the treatment of an HBV infection, and/or for decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In another aspect, a method of treating HBV infection and HBV-related symptoms in a subject in need thereof, comprising administering to the subject a combination of a LTα3 receptor agonist or a functional fragment thereof and an IFN agent selected from the group consisting of IFNγ and IFNλ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In a particular embodiment, a method of treating HBV infection and HBV-related symptoms in a subject in need thereof, comprising administering to the subject a combination of LTα3 or a functional fragment thereof and an IFN agent selected from the group consisting of IFNγ and IFNλ or a functional fragment thereof, to decrease one or more symptoms of HBV infection in the subject, is provided. According to the said embodiment, the said combination for use in the treatment of an HBV infection, and/or for decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In another aspect, a method of treating HBV infection and HBV-related symptoms in a subject in need thereof, comprising administering to the subject a combination of a tumor necrosis factor-like receptor weak inducer of apoptosis (TNFRSF12A) agonist or a functional fragment thereof; and an IFN agent selected from the group consisting of IFNα and IFNβ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating an HBV infection, and/or for decreasing one or more symptoms of HBV infection in the subject, is provided.

In a particular embodiment, a method of treating HBV infection and HBV-related symptoms in a subject in need thereof, comprising administering to the subject a combination of TWEAK or a functional fragment thereof; and an IFN agent selected from the group consisting of IFNα and IFNβ or a functional fragment thereof, to decrease one or more symptoms of HBV infection in the subject, is provided. According to the said embodiment, the said combination for use in the treatment of an HBV infection, and/or for decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In another aspect, a method of treating HBV infection and HBV-related symptoms in a subject in need thereof, comprising administering to the subject a combination of a tumor necrosis factor-like receptor weak inducer of apoptosis (TNFRSF12A) agonist or a functional fragment thereof; and an IFN agent selected from the group consisting of IFNγ and IFNλ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating an HBV infection, and/or for decreasing one or more symptoms of HBV infection in the subject, is provided.

In a particular embodiment, a method of treating HBV infection and HBV-related symptoms in a subject in need thereof, comprising administering to the subject a combination of TWEAK or a functional fragment thereof; and an IFN agent selected from the group consisting of IFNγ and IFNλ or a functional fragment thereof, to decrease one or more symptoms of HBV infection in the subject, is provided. According to the said embodiment, the said combination for use in the treatment of an HBV infection, and/or for decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In another aspect, a method of treating HBV infection and HBV-related symptoms in a subject in need thereof, comprising administering to the subject a combination of a LTβ receptor (TNFRSF3) agonist or a herpes virus entry mediator (HVEM or TNFRSF14) agonist or a functional fragment thereof; and an IFN agent selected from the group consisting of IFNα and IFNβ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating an HBV infection, and/or for decreasing one or more symptoms of HBV infection in the subject, is provided.

In a particular embodiment, a method of treating HBV infection and HBV-related symptoms in a subject in need thereof, comprising administering to the subject a combination of a LTβ receptor (TNFRSF3) agonist or a functional fragment thereof; and an IFN agent selected from the group consisting of IFNα and IFNβ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating an HBV infection, and/or for decreasing one or more symptoms of HBV infection in the subject, is provided.

In a particular embodiment, a method of treating HBV infection and HBV-related symptoms in a subject in need thereof, comprising administering to the subject a combination of a herpes virus entry mediator (HVEM or TNFRSF14) agonist or a functional fragment thereof and an IFN agent selected from the group consisting of IFNα and IFNβ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating an HBV infection, and/or for decreasing one or more symptoms of HBV infection in the subject, is provided.

In a particular embodiment, a method of treating HBV infection and HBV-related symptoms in a subject in need thereof, comprising administering to the subject a combination of LIGHT or a functional fragment thereof and an IFN agent selected from the group consisting of IFNα and IFNβ or a functional fragment thereof, to decrease one or more symptoms of HBV infection in the subject, is provided. According to the said embodiment, the said combination for use in the treatment of an HBV infection, and/or for decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In another aspect, a method of treating HBV infection and HBV-related symptoms in a subject in need thereof, comprising administering to the subject a combination of a LTβ receptor (TNFRSF3) agonist or a herpes virus entry mediator (HVEM or TNFRSF14) agonist or a functional fragment thereof and an IFN agent selected from the group consisting of IFNγ and IFNλ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating an HBV infection, and/or for decreasing one or more symptoms of HBV infection in the subject, is provided.

In a particular embodiment, a method of treating HBV infection and HBV-related symptoms in a subject in need thereof, comprising administering to the subject a combination of a LTβ receptor (TNFRSF3) agonist or a functional fragment thereof and an IFN agent selected from the group consisting of IFNγ and IFNλ or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating an HBV infection, and/or for decreasing one or more symptoms of HBV infection in the subject, is provided.

In a particular embodiment, a method of treating HBV infection and HBV-related symptoms in a subject in need thereof, comprising administering to the subject a combination of a herpes virus entry mediator (HVEM or TNFRSF14) agonist or a functional fragment thereof and an IFN agent selected from the group consisting of IFNγ and IFNλ, or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating an HBV infection, and/or for decreasing one or more symptoms of HBV infection in the subject, is provided.

In a particular embodiment, a method of treating HBV infection and HBV-related symptoms in a subject in need thereof, comprising administering to the subject a combination of LIGHT or a functional fragment thereof; and an IFN agent selected from the group consisting of IFNγ and IFNλ or a functional fragment thereof, to decrease one or more symptoms of HBV infection in the subject, is provided. According to the said embodiment, the said combination for use in the treatment of an HBV infection, and/or for decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In another aspect, method of treating an HBV infection and HBV-related symptoms in a subject in need thereof, comprising administering to the subject a pharmaceutical composition comprising a TNFRSF agonist or a functional fragment thereof, and administering to the subject a pharmaceutical composition comprising an IFN agent or a functional fragment thereof, to decrease one or more symptoms of HBV infection in the subject, is provided. According to the said aspect, the said combination for use in the treatment of an HBV infection, and/or for decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In another aspect, a TNFRSF agonist or a functional fragment thereof for use in treating one or more symptoms of HBV infection in a subject, in combination with an IFN agent or a functional fragment thereof, is provided. According to the said aspect, the use of the said combination for preparing a medicament for treating a HBV infection, and/or for decreasing one or more symptoms of HBV infection in the subject, is provided.

In another aspect, an IFN agent or a functional fragment thereof for use in treating one or more symptoms of HBV infection in a subject, in combination with a TNFRSF agonist or a functional fragment thereof, is provided. According to the said aspect, the use of the said combination for preparing a medicament for treating a HBV infection, and/or for decreasing one or more symptoms of HBV infection in the subject, is provided.

In another aspect, a combination of (i) at least one TNFRSF agonist or a functional fragment thereof and (ii) at least one IFN agent or a functional fragment thereof, for use in the treatment of HBV infection, is provided. According to the said aspect, the use of the said combination for preparing a medicament for treating a HBV infection, and/or for decreasing one or more symptoms of HBV infection in the subject, is provided.

In another aspect, a combination of (i) at least one TNFRSF agonist selected from the group consisting of a LTα3 receptor agonist, a LTβ (TNFRSF3) agonist, a herpes virus entry mediator (HVEM or TNFRSF14) agonist, a tumor necrosis factor-like receptor weak inducer of apoptosis (TNFRSF12A) agonist and a cluster of differentiation factor 40 (CD40, TNFRSF5) agonist or a functional fragment thereof and (ii) an IFN agent selected from the group consisting of a Type I IFN, a Type II IFN, and a Type III IFN or a functional fragment thereof, for use in the treatment of HBV infection, is provided. According to the said aspect, the use of the said combination for preparing a medicament for treating an HBV infection, and/or for decreasing one or more symptoms of HBV infection in the subject, is provided.

In another aspect, a combination of (i) at least one TNFRSF agonist selected from the group consisting of a LTα3 receptor agonist, a LTβ (TNFRSF3) agonist, a tumor necrosis factor-like receptor weak inducer of apoptosis (TNFRSF12A) agonist and a cluster of differentiation factor 40 (CD40, TNFRSF5) agonist or a functional fragment thereof and (ii) an IFN agent selected from the group consisting of a Type I IFN, a Type II IFN, and a Type III IFN or a functional fragment thereof, for use in the treatment of HBV infection, is provided. According to the said aspect, the use of the said combination for preparing a medicament for treating an HBV infection, and/or for decreasing one or more symptoms of HBV infection in the subject, is provided.

In another aspect, a combination of (i) at least one TNFRSF agonist selected from the group consisting of a LTα3 receptor agonist, a herpes virus entry mediator (HVEM or TNFRSF14) agonist, a tumor necrosis factor-like receptor weak inducer of apoptosis (TNFRSF12A) agonist and a cluster of differentiation factor 40 (CD40, TNFRSF5) agonist or a functional fragment thereof; and (ii) an IFN agent selected from the group consisting of a Type I IFN, a Type II IFN, and a Type III IFN or a functional fragment thereof, for use in the treatment of HBV infection, is provided. According to the said aspect, the use of the said combination for preparing a medicament for treating an HBV infection, and/or for decreasing one or more symptoms of HBV infection in the subject, is provided.

In another aspect, a combination of (i) at least one TNFRSF agonist selected from the group consisting of LTα3, TWEAK, LIGHT, and CD40L or a functional fragment thereof; and (ii) an IFN agent selected from the group consisting of a Type I IFN, a Type II IFN, and a Type III IFN or a functional fragment thereof, for use in the treatment of HBV infection, is provided. According to the said aspect, the use of the said combination for preparing a medicament for treating an HBV infection, and/or for decreasing one or more symptoms of HBV infection in the subject, is provided.

In another aspect, a combination of (i) at least one TNFRSF agonist selected from the group consisting of a LTα3 receptor agonist, a LTβ receptor (TNFRSF3) agonist, a herpes virus entry mediator (HVEM or TNFRSF14) agonist, a tumor necrosis factor-like receptor weak inducer of apoptosis (TNFRSF12A) agonist and a cluster of differentiation factor 40 (CD40, TNFRSF5) agonist or a functional fragment thereof; and (ii) an IFN agent selected from the group consisting of IFNα, IFNβ, IFNγ, and IFNλ or a functional fragment thereof, for use in the treatment of HBV infection, is provided. According to the said aspect, the use of the said combination for preparing a medicament for treating an HBV infection, and/or for decreasing one or more symptoms of HBV infection in the subject, is provided.

In a particular embodiment, a combination of (i) at least one TNFRSF agonist selected from the group consisting of a LTα3 receptor agonist, a receptor LTβ (TNFRSF3) agonist, a tumor necrosis factor-like receptor weak inducer of apoptosis (TNFRSF12A) agonist and a cluster of differentiation factor 40 (CD40, TNFRSF5) agonist or a functional fragment thereof; and (ii) an IFN agent selected from the group consisting of IFNα, IFNβ, IFNγ, and IFNλ or a functional fragment thereof, for use in the treatment of HBV infection, is provided. According to the said aspect, the use of the said combination for preparing a medicament for treating an HBV infection, and/or for decreasing one or more symptoms of HBV infection in the subject, is provided.

In a particular embodiment, a combination of (i) at least one TNFRSF agonist selected from the group consisting of a LTα3 receptor agonist, a herpes virus entry mediator (HVEM or TNFRSF14) agonist, a tumor necrosis factor-like receptor weak inducer of apoptosis (TNFRSF12A) agonist and a cluster of differentiation factor 40 (CD40, TNFRSF5) agonist or a functional fragment thereof; and (ii) an IFN agent selected from the group consisting of IFNα, IFNβ, IFNγ, and IFNλ or a functional fragment thereof, for use in the treatment of HBV infection, is provided. According to the said aspect, the use of the said combination for preparing a medicament for treating an HBV infection, and/or for decreasing one or more symptoms of HBV infection in the subject, is provided.

In a particular embodiment, a combination of (i) at least one TNFRSF agonist selected from the group consisting of LTα3, TWEAK, LIGHT, and CD40L or a functional fragment thereof; and (ii) an IFN agent selected from the group consisting of IFNα, IFNβ, IFNγ, and IFNλ or a functional fragment thereof, for use in the treatment of HBV infection, is provided. According to the said embodiment, the use of the said combination for preparing a medicament for treating a HBV infection, and/or for decreasing one or more symptoms of HBV infection in the subject, is provided.

In another aspect, a combination of (i) at least one TNFRSF agonist selected from the group consisting of a LTβ receptor (TNFRSF3) agonist, a herpes virus entry mediator (HVEM or TNFRSF14) agonist, a tumor necrosis factor-like receptor weak inducer of apoptosis (TNFRSF12A) agonist and a cluster of differentiation factor 40 (CD40, TNFRSF5) agonist or a functional fragment thereof and (ii) an IFN agent selected from the group consisting of IFNα, IFNβ, IFNγ, and IFNλ or a functional fragment thereof, for use in the treatment of HBV infection, is provided. According to the said aspect, the use of the said combination for preparing a medicament for treating an HBV infection, and/or for decreasing one or more symptoms of HBV infection in the subject, is provided.

In a particular embodiment, a combination of (i) at least one TNFRSF agonist selected from the group consisting of a LTβ receptor (TNFRSF3) agonist, a tumor necrosis factor-like receptor weak inducer of apoptosis (TNFRSF12A) agonist and a cluster of differentiation factor 40 (CD40, TNFRSF5) agonist or a functional fragment thereof and (ii) an IFN agent selected from the group consisting of IFNα, IFNβ, IFNγ, and IFNλ or a functional fragment thereof, for use in the treatment of HBV infection, is provided. According to the said aspect, the use of the said combination for preparing a medicament for treating an HBV infection, and/or for decreasing one or more symptoms of HBV infection in the subject, is provided.

In a particular embodiment, a combination of (i) at least one TNFRSF agonist selected from the group consisting of a herpes virus entry mediator (HVEM or TNFRSF14) agonist, a tumor necrosis factor-like receptor weak inducer of apoptosis (TNFRSF12A) agonist and a cluster of differentiation factor 40 (CD40, TNFRSF5) agonist or a functional fragment thereof; and (ii) an IFN agent selected from the group consisting of IFNα, IFNβ, IFNγ, and IFNλ or a functional fragment thereof, for use in the treatment of HBV infection, is provided. According to the said aspect, the use of the said combination for preparing a medicament for treating an HBV infection, and/or for decreasing one or more symptoms of HBV infection in the subject, is provided.

In a particular embodiment, a combination of (i) at least one TNFRSF agonist selected from the group consisting of TWEAK, LIGHT, and CD40L or a functional fragment thereof and (ii) an IFN agent selected from the group consisting of IFNα, IFNβ, IFNγ, and IFNλ or a functional fragment thereof, for use in the treatment of HBV infection, is provided. According to the said embodiment, the use of the said combination for preparing a medicament for treating an HBV infection, and/or for decreasing one or more symptoms of HBV infection in the subject, is provided.

In another aspect, a combination of (i) at least one TNFRSF agonist selected from the group consisting of a LTα3 receptor agonist, a receptor LTβ (TNFRSF3) agonist, a herpes virus entry mediator (HVEM or TNFRSF14) agonist and a tumor necrosis factor-like receptor weak inducer of apoptosis (TNFRSF12A) agonist or a functional fragment thereof and (ii) an IFN agent selected from the group consisting of IFNα and IFNβ or a functional fragment thereof, for use in the treatment of HBV infection, is provided. According to the said aspect, the use of the said combination for preparing a medicament for treating an HBV infection, and/or for decreasing one or more symptoms of HBV infection in the subject, is provided.

In a particular embodiment, a combination of (i) at least one TNFRSF agonist selected from the group consisting of a LTα3 receptor agonist, a receptor LTβ (TNFRSF3) agonist, and a tumor necrosis factor-like receptor weak inducer of apoptosis (TNFRSF12A) agonist or a functional fragment thereof and (ii) an IFN agent selected from the group consisting of IFNα and IFNβ or a functional fragment thereof, for use in the treatment of HBV infection, is provided. According to the said aspect, the use of the said combination for preparing a medicament for treating an HBV infection, and/or for decreasing one or more symptoms of HBV infection in the subject, is provided.

In a particular embodiment, a combination of (i) at least one TNFRSF agonist selected from the group consisting of a LTα3 receptor agonist, a herpes virus entry mediator (HVEM or TNFRSF14) agonist and a tumor necrosis factor-like receptor weak inducer of apoptosis (TNFRSF12A) agonist or a functional fragment thereof and (ii) an IFN agent selected from the group consisting of IFNα and IFNβ or a functional fragment thereof, for use in the treatment of HBV infection, is provided. According to the said aspect, the use of the said combination for preparing a medicament for treating an HBV infection, and/or for decreasing one or more symptoms of HBV infection in the subject, is provided

In a particular embodiment, a combination of (i) at least one TNFRSF agonist selected from the group consisting of LTα3, TWEAK, and LIGHT or a functional fragment thereof and (ii) an IFN agent selected from the group consisting of IFNα and IFNβ or a functional fragment thereof, for use in the treatment of HBV infection, is provided. According to the said embodiment, the use of the said combination for preparing a medicament for treating a HBV infection, and/or for decreasing one or more symptoms of HBV infection in the subject, is provided.

In another aspect, a combination of (i) at least one TNFRSF agonist selected from the group consisting of a LTβ receptor (TNFRSF3) agonist, a herpes virus entry mediator (HVEM or TNFRSF14) agonist, a tumor necrosis factor-like receptor weak inducer of apoptosis (TNFRSF12A) agonist and a cluster of differentiation factor 40 (CD40, TNFRSF5) agonist or a functional fragment thereof and (ii) an IFN agent selected from the group consisting of IFNα and IFNβ or a functional fragment thereof, for use in the treatment of HBV infection, is provided. According to the said aspect, the use of the said combination for preparing a medicament for treating an HBV infection, and/or for decreasing one or more symptoms of HBV infection in the subject, is provided.

In a particular embodiment, a combination of (i) at least one TNFRSF agonist selected from the group consisting of a LTβ receptor (TNFRSF3) agonist, a tumor necrosis factor-like receptor weak inducer of apoptosis (TNFRSF12A) agonist and a cluster of differentiation factor 40 (CD40, TNFRSF5) agonist or a functional fragment thereof; and (ii) an IFN agent selected from the group consisting of IFNα and IFNβ or a functional fragment thereof, for use in the treatment of HBV infection, is provided. According to the said aspect, the use of the said combination for preparing a medicament for treating an HBV infection, and/or for decreasing one or more symptoms of HBV infection in the subject, is provided.

In a particular embodiment, a combination of (i) at least one TNFRSF agonist selected from the group consisting of a herpes virus entry mediator (HVEM or TNFRSF14) agonist, a tumor necrosis factor-like receptor weak inducer of apoptosis (TNFRSF12A) agonist and a cluster of differentiation factor 40 (CD40, TNFRSF5) agonist or a functional fragment thereof; and (ii) an IFN agent selected from the group consisting of IFNα and IFNβ or a functional fragment thereof, for use in the treatment of HBV infection, is provided. According to the said aspect, the use of the said combination for preparing a medicament for treating an HBV infection, and/or for decreasing one or more symptoms of HBV infection in the subject, is provided.

In a particular embodiment, a combination of (i) at least one TNFRSF agonist selected from the group consisting of TWEAK, LIGHT and CD40L or a functional fragment thereof; and (ii) an IFN agent selected from the group consisting of IFNα and IFNβ or a functional fragment thereof, for use in the treatment of HBV infection, is provided. According to the said embodiment, the use of the said combination for preparing a medicament for treating an HBV infection, and/or for decreasing one or more symptoms of HBV infection in the subject, is provided.

In another aspect, a combination of (i) at least one TNFRSF agonist selected from the group consisting of LTα3 receptor agonist, a LTβ receptor (TNFRSF3) agonist, a herpes virus entry mediator (HVEM or TNFRSF14) agonist and a tumor necrosis factor-like receptor weak inducer of apoptosis (TNFRSF12A) agonist or a functional fragment thereof; and (ii) an IFN agent selected from the group consisting of IFNγ and IFNλ or a functional fragment thereof, for use in the treatment of HBV infection, is provided. According to the said aspect, the use of the said combination for preparing a medicament for treating an HBV infection, and/or for decreasing one or more symptoms of HBV infection in the subject, is provided.

In a particular embodiment, a combination of (i) at least one TNFRSF agonist selected from the group consisting of LTα3 receptor agonist, a LTβ receptor (TNFRSF3) agonist, and a tumor necrosis factor-like receptor weak inducer of apoptosis (TNFRSF12A) agonist or a functional fragment thereof; and (ii) an IFN agent selected from the group consisting of IFNγ and IFNλ or a functional fragment thereof, for use in the treatment of HBV infection, is provided. According to the said aspect, the use of the said combination for preparing a medicament for treating an HBV infection, and/or for decreasing one or more symptoms of HBV infection in the subject, is provided.

In a particular embodiment, a combination of (i) at least one TNFRSF agonist selected from the group consisting of LTα3 receptor agonist, a herpes virus entry mediator (HVEM or TNFRSF14) agonist and a tumor necrosis factor-like receptor weak inducer of apoptosis (TNFRSF12A) agonist or a functional fragment thereof; and (ii) an IFN agent selected from the group consisting of IFNγ and IFNλ or a functional fragment thereof, for use in the treatment of HBV infection, is provided. According to the said aspect, the use of the said combination for preparing a medicament for treating an HBV infection, and/or for decreasing one or more symptoms of HBV infection in the subject, is provided.

In a particular embodiment, a combination of (i) at least one TNFRSF agonist selected from the group consisting of LTα3, TWEAK, and LIGHT or a functional fragment thereof; and (ii) an IFN agent selected from the group consisting of IFNγ and IFNλ or a functional fragment thereof, for use in the treatment of HBV infection, is provided. According to the said embodiment, the use of the said combination for preparing a medicament for treating a HBV infection, and/or for decreasing one or more symptoms of HBV infection in the subject, is provided.

In another aspect, a combination of (i) at least one TNFRSF agonist selected from the group consisting of a LTβ receptor (TNFRSF3) agonist, a herpes virus entry mediator (HVEM or TNFRSF14) agonist, a tumor necrosis factor-like receptor weak inducer of apoptosis (TNFRSF12A) agonist and a cluster of differentiation factor 40 (CD40, TNFRSF5) agonist or a functional fragment thereof and (ii) an IFN agent selected from the group consisting of IFNγ and IFNλ or a functional fragment thereof, for use in the treatment of HBV infection, is provided. According to the said aspect, the use of the said combination for preparing a medicament for treating an HBV infection, and/or for decreasing one or more symptoms of HBV infection in the subject, is provided.

In a particular embodiment, combination of (i) at least one TNFRSF agonist selected from the group consisting of a LTβ receptor (TNFRSF3) agonist, a tumor necrosis factor-like receptor weak inducer of apoptosis (TNFRSF12A) agonist and a cluster of differentiation factor 40 (CD40, TNFRSF5) agonist or a functional fragment thereof and (ii) an IFN agent selected from the group consisting of IFNγ and IFNλ or a functional fragment thereof, for use in the treatment of HBV infection, is provided. According to the said aspect, the use of the said combination for preparing a medicament for treating an HBV infection, and/or for decreasing one or more symptoms of HBV infection in the subject, is provided.

In a particular embodiment, combination of (i) at least one TNFRSF agonist selected from the group consisting of a herpes virus entry mediator (HVEM or TNFRSF14) agonist, a tumor necrosis factor-like receptor weak inducer of apoptosis (TNFRSF12A) agonist and a cluster of differentiation factor 40 (CD40, TNFRSF5) agonist or a functional fragment thereof; and (ii) an IFN agent selected from the group consisting of IFNγ and IFNλ or a functional fragment thereof, for use in the treatment of HBV infection, is provided. According to the said aspect, the use of the said combination for preparing a medicament for treating an HBV infection, and/or for decreasing one or more symptoms of HBV infection in the subject, is provided.

In a particular embodiment, a combination of (i) at least one TNFRSF agonist selected from the group consisting of TWEAK, LIGHT and CD40L or a functional fragment thereof and (ii) an IFN agent selected from the group consisting of IFNγ and IFNλ or a functional fragment thereof, for use in the treatment of HBV infection, is provided. According to the said embodiment, the use of the said combination for preparing a medicament for treating an HBV infection, and/or for decreasing one or more symptoms of HBV infection in the subject, is provided.

In another aspect, a combination of (i) at least one TNFRSF agonist or a functional fragment thereof, and (ii) at least an IFN agent or a functional fragment thereof, for use in the treatment of HBV infection by reducing the amount of pre-genomic HBV RNA in an HBV-infected cell, is provided. According to the said aspect, the use of the said combination for preparing a medicament for treating a HBV infection, and/or for decreasing one or more symptoms of HBV infection in the subject, is provided.

In another aspect, a method of treating a HBV infection in a subject in need thereof, comprising administering to the subject a combination of a TNFRSF agonist and an IFN agent or a functional fragment thereof, to decrease one or more symptoms of HBV infection in the subject, is provided.

In an embodiment of any one of the aspects of the invention or in combination with any other embodiment of the invention, transcription of covalently closed circular (ccc) DNA to generate pregenomic (pg) RNA is inhibited in infected cells of the subject.

In an embodiment of any one of the aspects of the invention or in combination with any other embodiment of the invention, the combination of the TNFRSF agonist and the IFN agent or functional fragment thereof synergistically inhibits transcription of pgRNA in infected cells of the subject.

In an embodiment of any one of the aspects of the invention or in combination with any other embodiment of the invention, hepatitis B e-antigen (HBeAg) release from HBV-infected cells in the subject is inhibited.

In an embodiment of any one of the aspects of the invention or in combination with any other embodiment of the invention, the combination of the TNFRSF agonist and the IFN agent or functional fragment thereof synergistically inhibits HBeAg release from infected cells.

In an embodiment of any one of the aspects of the invention or in combination with any other embodiment of the invention, the combination of the TNFRSF agonist and the IFN agent or functional fragment thereof stimulates the IFN pathway in the subject.

In an embodiment of any one of the aspects of the invention, an IFN pathway biomarker level is increased in the subject, wherein said biomarker is selected from the group consisting of C-X-C motif chemokine 9 (CXCL9), C-X-C motif chemokine 10 (CXCL10) and C-X-C motif chemokine 11 (CXCL11).

In a particular embodiment, the combination of the TNFRSF agonist and the IFN agent or functional fragment thereof synergistically increases the IFN pathway biomarker level in the subject.

In an embodiment of any one of the aspects of the invention, the biomarker is C-X-C motif chemokine 10 (CXCL10).

In a particular embodiment, the combination of the TNFRSF agonist and the IFN agent or functional fragment thereof synergistically increases the CXCL10 biomarker level in the subject.

In an embodiment of any one of the aspects of the invention, the combination of the TNFRSF agonist and the IFN agent or functional fragment thereof synergistically stimulates CXCL10 release in the subject.

In an embodiment of any one of the aspects of the invention, the infected cells are hepatocytes.

In an embodiment of any one of the aspects of the invention, the TNFRSF agonist is selected from the group consisting of a LTα3 receptor (TNFRSF1A, TNFRSF1B, or TNFRSF14) agonist (e.g. LTα3), a LTβ receptor (TNFRSF3) agonist (e.g., LIGHT or LTβ), a herpesvirus entry mediator (HVEM or TNFRSF14) agonist (e.g. LIGHT), a tumor necrosis factor-like receptor weak inducer of apoptosis (TNFRSF12A) agonist (e.g., TWEAK also known as TNFSF12), a cluster of differentiation factor 40 (CD40, TNFRSF5) agonist (e.g. CD40L), a CD27 (TNFRSF7) agonist (e.g. CD70), a CD30 (TNFRSF8) agonist, a 4-1BB (CD137, TNFRSF9) agonist, a receptor activator of nuclear factor κB (RANK, TNFRSF11A) agonist, a Troy (TNFRSF19) agonist, and a OX40 receptor (TNFRSF4) agonist.

In an embodiment of any one of the aspects of the invention, the TNFRSF agonist is selected from the group consisting of a lymphotoxin alpha 3 (LTα3) receptor (TNFRSF1A, TNFRSF1B, or TNFRSF14) agonist, a lymphotoxin beta LTβ receptor (TNFRSF3) agonist (e.g., LIGHT or LTβ), a herpesvirus entry mediator (HVEM or TNFRSF14) agonist (LIGHT), a tumor necrosis factor-like receptor weak inducer of apoptosis (TNFRSF12A) agonist (e.g., TWEAK also known as TNFSF12), a cluster of differentiation factor 40 (CD40, TNFRSF5) agonist (CD40L), a CD27 (TNFRSF7) agonist, a CD30 (TNFRSF8) agonist, a 4-1BB (CD137, TNFRSF9) agonist, a receptor activator of nuclear factor κB (RANK, TNFRSF11A) agonist, a Troy (TNFRSF19) agonist, and a OX40 receptor (TNFRSF4) agonist.

In an embodiment of any one of the aspects of the invention, the TNFRSF agonist is selected from the group consisting of a lymphotoxin alpha 3 (LTα3) receptor (TNFRSF1A, TNFRSF1B, or TNFRSF14) agonist (e.g. LTα3), a lymphotoxin beta LTβ receptor (TNFRSF3) agonist (e.g., LIGHT or LTβ), a herpesvirus entry mediator (HVEM or TNFRSF14) agonist (e.g. LIGHT), a tumor necrosis factor-like receptor weak inducer of apoptosis (TNFRSF12A) agonist (e.g., TWEAK also known as TNFSF12), a cluster of differentiation factor 40 (CD40, TNFRSF5) agonist (e.g. CD40L), a CD27 (TNFRSF7) agonist (e.g. CD70), a CD30 (TNFRSF8) agonist, a receptor activator of nuclear factor κB (RANK, TNFRSF11A) agonist, a Troy (TNFRSF19) agonist, and a OX40 receptor (TNFRSF4) agonist.

In an embodiment of any one of the aspects of the invention, the TNFRSF agonist is selected from the group consisting of a lymphotoxin alpha 3 (LTα3) receptor (TNFRSF1A, TNFRSF1B, or TNFRSF14) agonist, a lymphotoxin beta LTβ receptor (TNFRSF3) agonist (e.g., LIGHT or LTβ), a herpesvirus entry mediator (HVEM or TNFRSF14) agonist (LIGHT), a tumor necrosis factor-like receptor weak inducer of apoptosis (TNFRSF12A) agonist (e.g., TWEAK also known as TNFSF12), a cluster of differentiation factor 40 (CD40, TNFRSF5) agonist (CD40L), a CD27 (TNFRSF7) agonist, a CD30 (TNFRSF8) agonist, a receptor activator of nuclear factor κB (RANK, TNFRSF11A) agonist, a Troy (TNFRSF19) agonist, and a OX40 receptor (TNFRSF4) agonist.

The TNFRSF agonist or functional fragment thereof according to the invention may be provided as a fusion protein comprising said TNFRSF agonist or a functional fragment thereof.

In an embodiment of any one of the aspects of the invention, the TNFRSF agonist is a CD40 agonist selected from the group consisting of a CD40 ligand (CD40L), or a functional fragment thereof, an agonistic anti-CD40 antibody or antigen-binding fragment thereof, and a fusion protein comprising a CD40 ligand, or a functional fragment thereof.

In an embodiment of any one of the aspects of the invention, the TNFRSF agonist comprises or consists of TWEAK, or a functional fragment thereof. In particular, the TNFRSF agonist is a fusion protein comprising TWEAK or a functional fragment thereof.

In an embodiment of any one of the aspects of the invention, the TNFRSF agonist comprises or consists of LIGHT, or a functional fragment thereof. In particular, the TNFRSF agonist is a fusion protein comprising LIGHT or a functional fragment thereof.

In an embodiment of any one of the aspects of the invention, the TNFRSF agonist comprises or consists of CD40L, or a functional fragment thereof. In particular, the TNFRSF agonist is a fusion protein comprising CD40L or a functional fragment thereof.

In an embodiment of any one of the aspects of the invention, the CD40L is hexameric CD40L or trimeric CD40L.

In an embodiment of any one of the aspects of the invention, the fusion protein is a bifunctional immunostimulatory fusion protein comprising a TNFRSF agonist or a functional fragment thereof, an IFN agent or a functional fragment thereof and a linker.

According to a particular embodiment, the TNFRSF agonist or a functional fragment thereof and the IFN agent or a functional fragment thereof are provided as a bifunctional immunostimulatory fusion protein comprising the said TNFRSF agonist or a functional fragment thereof, an IFN agent or a functional fragment thereof and a linker.

In an embodiment of any one of the aspects of the invention, the IFN agent or functional fragment thereof is selected from the group consisting of a Type I IFN, a Type II IFN and a Type III IFN.

In an embodiment of any one of the aspects of the invention, the IFN agent or functional fragment thereof is IFNα, IFNβ, IFNγ or IFNλ.

In an embodiment of any one of the aspects of the invention, the IFN agent or functional fragment thereof is IFNβ or IFNγ.

According to a particular embodiment, the IFN agent or functional fragment thereof is provided as a fusion protein comprising said IFN agent or functional fragment thereof.

In an embodiment of any one of the aspects of the invention, the IFN agent or functional fragment thereof is IFNβ. In particular, IFNβ is provided as a fusion protein comprising IFNβ or a functional fragment thereof.

In an embodiment of any one of the aspects of the invention, the IFN agent or functional fragment thereof is IFNα. In particular, IFNα is provided as a fusion protein comprising IFNα or a functional fragment thereof.

In an embodiment of any one of the aspects of the invention, the IFN agent or functional fragment thereof is IFNλ. In particular, IFNλ is provided as a fusion protein comprising IFNλ, or a functional fragment thereof.

In an embodiment of any one of the aspects of the invention, the IFN agent or functional fragment thereof is IFNγ. In particular, IFNγ is provided as a fusion protein comprising IFNγ or a functional fragment thereof.

In an embodiment of any one of the aspects of the invention, the IFN agent is provided as a fusion protein.

In an embodiment of any one of the aspects of the invention, the fusion protein is a bifunctional immunostimulatory fusion protein.

In an embodiment of any one of the aspects of the invention, the bifunctional immunostimulatory fusion protein further comprises a TNFRSF agonist or functional fragment thereof.

In an embodiment of any one of the aspects of the invention, the TNFRSF agonist or functional fragment thereof is selected from the group consisting of CD40L, LTα3, LIGHT and/or TWEAK.

In an embodiment of any one of the aspects of the invention, the TNFRSF agonist or functional fragment thereof is selected from the group consisting of CD40L, LIGHT and/or TWEAK.

In an embodiment of any one of the aspects of the invention, the TNFRSF agonist is a polypeptide, an antibody, or an antigen-binding fragment thereof.

In an embodiment of any one of the aspects of the invention, the TNFRSF polypeptide, antibody or antigen-binding fragment thereof is provided directly to the subject.

In an embodiment of any one of the aspects of the invention, the TNFRSF polypeptide, antibody or antigen-binding fragment thereof is provided as a bifunctional immunostimulatory fusion protein.

In an embodiment of any one of the aspects of the invention, the TNFRSF polypeptide, antibody or antigen-binding fragment thereof is expressed from a polynucleotide provided to the subject.

In an embodiment of any one of the aspects of the invention, the IFN agent or functional fragment thereof is provided directly to the subject.

In an embodiment of any one of the aspects of the invention, the IFN agent or functional fragment thereof is provided as a bifunctional immunostimulatory fusion protein.

In an embodiment of any one of the aspects of the invention, the IFN agent or functional fragment thereof is expressed from a polynucleotide provided to the subject.

In another aspect, a method of treating HBV infection in a subject in need thereof, comprising administering to the subject with a combination of: (i) a lymphotoxin alpha 3 (LTα3) receptor (TNFRSF1A, TNFRSF1B, or TNFRSF14) agonist, a lymphotoxin beta (LTβ) receptor (TNFRSF3) agonist (e.g., LIGHT or LTβ), a herpesvirus entry mediator (HVEM or TNFRSF14) agonist (LIGHT), a tumor necrosis factor-like receptor weak inducer of apoptosis (TNFRSF12A) agonist (e.g., TWEAK, also known as TNFSF12), a cluster of differentiation factor 40 (CD40, TNFRSF5) agonist (CD40L), a CD27 (TNFRSF7) agonist (CD70), a CD30 (TNFRSF8) agonist, a 4-1BB (CD137, TNFRSF9) agonist, a receptor activator of nuclear factor κB (RANK, TNFRSF11A) agonist, a Troy (TNFRSF19) agonist, and a OX40 receptor (TNFRSF4) agonist; and (ii) an IFN agent or a functional fragment thereof, to decrease one or more symptoms of HBV infection in the subject, is provided.

In another aspect, a method of treating HBV infection in a subject in need thereof, comprising administering to the subject a combination of: a TNFRSF agonist; and a Type I IFN, or a functional fragment of a Type I IFN to decrease one or more symptoms of HBV infection in the subject, is provided.

In another aspect, a method of treating HBV infection in a subject in need thereof, comprising administering to the subject a combination of: a TNFRSF agonist; and a Type II IFN, a functional fragment of a Type II IFN, a Type III IFN, or a functional fragment of a Type III IFN, to decrease one or more symptoms of HBV infection in the subject, is provided.

In another aspect, a method of treating HBV infection in a subject in need thereof, comprising administering to the subject a combination of: LTα3, LIGHT and/or TWEAK; and an IFN agent or a functional fragment thereof, to decrease one or more symptoms of HBV infection in the subject, is provided.

In another aspect, a method of treating HBV infection in a subject in need thereof, comprising administering to the subject a combination of: CD40L; and an IFN agent or a functional fragment thereof, to decrease one or more symptoms of HBV infection in the subject, is provided.

In another aspect, a method of treating HBV infection in a subject in need thereof, comprising administering to the subject a combination of: a TNFRSF agonist; and IFNα or a functional fragment thereof, to decrease one or more symptoms of HBV infection in the subject, is provided.

In another aspect, a method of treating HBV infection in a subject in need thereof, comprising administering to the subject a combination of: LTα3, LIGHT and/or TWEAK; and IFNα or a functional fragment thereof, to decrease one or more symptoms of HBV infection in the subject, is provided.

In another aspect, a method of treating HBV infection in a subject in need thereof, comprising administering to the subject a combination of: CD40L; and IFNα or a functional fragment thereof, to decrease one or more symptoms of HBV infection in the subject, is provided.

In another aspect, a method of treating HBV infection in a subject in need thereof, comprising administering to the subject a combination of: a TNFRSF agonist; and IFNβ or a functional fragment thereof, to decrease one or more symptoms of HBV infection in the subject, is provided.

In another aspect, a method of treating HBV infection in a subject in need thereof, comprising administering to the subject a combination of: LTα3, LIGHT and/or TWEAK; and IFNβ or a functional fragment thereof, to decrease one or more symptoms of HBV infection in the subject, is provided.

In another aspect, a method of treating HBV infection in a subject in need thereof, comprising administering to the subject a combination of: CD40L; and IFNβ or a functional fragment thereof, to decrease one or more symptoms of HBV infection in the subject, is provided.

In another aspect, a method of treating HBV infection in a subject in need thereof, comprising administering to the subject a combination of: a TNFRSF agonist; and IFNγ or a functional fragment thereof, to decrease one or more symptoms of HBV infection in the subject, is provided.

In another aspect, a method of treating HBV infection in a subject in need thereof, comprising administering to the subject a combination of: LTα3, LIGHT and/or TWEAK; and IFNγ or a functional fragment thereof, to decrease one or more symptoms of HBV infection in the subject, is provided.

In another aspect, a method of treating HBV infection in a subject in need thereof, comprising administering to the subject a combination of: CD40L; and IFNγ or a functional fragment thereof, to decrease one or more symptoms of HBV infection in the subject, is provided.

In another aspect, a method of treating HBV infection in a subject in need thereof, comprising administering to the subject a combination of: a TNFRSF agonist; and IFNλ or a functional fragment thereof, to decrease one or more symptoms of HBV infection in the subject, is provided.

In another aspect, a method of treating HBV infection in a subject in need thereof, comprising administering to the subject a combination of: LTα3, LIGHT and/or TWEAK; and IFNλ or a functional fragment thereof, to decrease one or more symptoms of HBV infection in the subject, is provided.

In another aspect, a method of treating HBV infection in a subject in need thereof, comprising administering to the subject a combination of: CD40L; and IFNλ or a functional fragment thereof, to decrease one or more symptoms of HBV infection in the subject, is provided.

In another aspect of the invention, a method of treating an HBV infection in a subject in need thereof, comprising: administering to the subject a pharmaceutical composition comprising a TNFRSF agonist; and administering to the subject a pharmaceutical composition comprising an IFN agent or a functional fragment thereof, to decrease one or more symptoms of HBV infection in the subject, is provided.

In an embodiment of any one of the aspects of the invention, the pharmaceutical compositions are administered sequentially.

In an embodiment of any one of the aspects of the invention, the pharmaceutical compositions are administered concomitantly.

In an embodiment of any one of the aspects of the invention, the TNFRSF agonist and IFN agent or functional fragment thereof are administered in a single pharmaceutical composition.

In an embodiment of any one of the aspects of the invention, the TNFRSF agonist and IFN agent or functional fragment thereof are comprised in distinct pharmaceutical compositions.

In another aspect of the invention, a TNFRSF agonist for use in treating one or more symptoms of HBV infection in a subject, in combination with an IFN agent or a functional fragment thereof, is provided.

In another aspect of the invention, an IFN agent or a functional fragment thereof for use in treating one or more symptoms of HBV infection in a subject, in combination with a TNFRSF agonist, is provided.

In another aspect of the invention, a method of treating HBV infection in a subject in need thereof, comprising administering to the subject a combination of a cluster of differentiation factor 40 (CD40, TNFRSF5) agonist and IFNα or a functional fragment thereof to decrease one or more symptoms of HBV infection in the subject, is provided. According to the said aspect, the said combination for use in the treatment of an HBV infection, and/or decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In a particular embodiment, a method of treating HBV infection in a subject in need thereof, comprising administering to the subject a combination of CD40L and IFNα or a functional fragment thereof to decrease one or more symptoms of HBV infection in the subject, is provided. According to the said embodiment, the said combination for use in the treatment of an HBV infection, and/or decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In another aspect of the invention, a method of treating HBV infection in a subject in need thereof, comprising administering to the subject a combination of a cluster of differentiation factor 40 (CD40, TNFRSF5) agonist and IFNβ or a functional fragment thereof to decrease one or more symptoms of HBV infection in the subject, is provided. According to the said aspect, the said combination for use in the treatment of an HBV infection, and/or decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In a particular embodiment, a method of treating HBV infection in a subject in need thereof, comprising administering to the subject a combination of CD40L and IFNβ or a functional fragment thereof to decrease one or more symptoms of HBV infection in the subject, is provided. According to the said embodiment, the said combination for use in the treatment of an HBV infection, and/or decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In another aspect of the invention, a method of treating HBV infection in a subject in need thereof, comprising administering to the subject a combination of a cluster of differentiation factor 40 (CD40, TNFRSF5) agonist and INFγ or a functional fragment thereof to decrease one or more symptoms of HBV infection in the subject, is provided. According to the said aspect, the said combination for use in the treatment of an HBV infection, and/or decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In a particular embodiment, a method of treating HBV infection in a subject in need thereof, comprising administering to the subject a combination of a cluster of differentiation factor 40 (CD40, TNFRSF5) agonist and IL28 or a functional fragment thereof to decrease one or more symptoms of HBV infection in the subject, is provided. According to the said embodiment, the said combination for use in the treatment of an HBV infection, and/or decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In a particular embodiment, a method of treating HBV infection in a subject in need thereof, comprising administering to the subject a combination of CD40L and IL28 or a functional fragment thereof to decrease one or more symptoms of HBV infection in the subject, is provided. According to the said embodiment, the said combination for use in the treatment of an HBV infection, and/or decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In a particular embodiment, a method of treating HBV infection in a subject in need thereof, comprising administering to the subject a combination of a cluster of differentiation factor 40 (CD40, TNFRSF5) agonist and IL29 or a functional fragment thereof to decrease one or more symptoms of HBV infection in the subject, is provided. According to the said embodiment, the said combination for use in the treatment of an HBV infection, and/or decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In a particular embodiment, a method of treating HBV infection in a subject in need thereof, comprising administering to the subject a combination of CD40L and IL29 or a functional fragment thereof to decrease one or more symptoms of HBV infection in the subject, is provided. According to the said embodiment, the said combination for use in the treatment of an HBV infection, and/or decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In another aspect of the invention, a method of treating HBV infection in a subject in need thereof, comprising administering to the subject a combination of a LTα3 receptor agonist and IFNα or a functional fragment thereof to decrease one or more symptoms of HBV infection in the subject, is provided. According to the said aspect, the said combination for use in the treatment of an HBV infection, and/or decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In a particular embodiment, a method of treating HBV infection in a subject in need thereof, comprising administering to the subject a combination of LTα3 and IFNα or a functional fragment thereof to decrease one or more symptoms of HBV infection in the subject, is provided. According to the said embodiment, the said combination for use in the treatment of an HBV infection, and/or decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In another aspect of the invention, a method of treating HBV infection in a subject in need thereof, comprising administering to the subject a combination of a tumor necrosis factor-like receptor weak inducer of apoptosis (TNFRSF12A) agonist and IFNα or a functional fragment thereof to decrease one or more symptoms of HBV infection in the subject, is provided. According to the said aspect, the said combination for use in the treatment of an HBV infection, and/or decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In a particular embodiment, a method of treating HBV infection in a subject in need thereof, comprising administering to the subject a combination of TWEAK and IFNα or a functional fragment thereof to decrease one or more symptoms of HBV infection in the subject, is provided. According to the said embodiment, the said combination for use in the treatment of an HBV infection, and/or decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In another aspect of the invention, a method of treating HBV infection in a subject in need thereof, comprising administering to the subject a combination of a LTβ receptor (TNFRSF3) agonist or a herpesvirus entry mediator (HVEM or TNFRSF14) agonist and IFNα or a functional fragment thereof to decrease one or more symptoms of HBV infection in the subject, is provided. According to the said aspect, the said combination for use in the treatment of an HBV infection, and/or decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In a particular embodiment, a method of treating HBV infection in a subject in need thereof, comprising administering to the subject a combination of a LTβ receptor (TNFRSF3) agonist and IFNα or a functional fragment thereof to decrease one or more symptoms of HBV infection in the subject, is provided. According to the said aspect, the said combination for use in the treatment of an HBV infection, and/or decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In particular embodiment, a method of treating HBV infection in a subject in need thereof, comprising administering to the subject a combination of a herpesvirus entry mediator (HVEM or TNFRSF14) agonist and IFNα or a functional fragment thereof to decrease one or more symptoms of HBV infection in the subject, is provided. According to the said aspect, the said combination for use in the treatment of an HBV infection, and/or decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In a particular embodiment, a method of treating HBV infection in a subject in need thereof, comprising administering to the subject a combination of LIGHT and IFNα or a functional fragment thereof to decrease one or more symptoms of HBV infection in the subject, is provided. According to the said embodiment, the said combination for use in the treatment of an HBV infection, and/or decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In another aspect of the invention, a method of treating HBV infection in a subject in need thereof, comprising administering to the subject a combination of a LTα3 receptor agonist and IFNβ or a functional fragment thereof to decrease one or more symptoms of HBV infection in the subject, is provided. According to the said aspect, the said combination for use in the treatment of an HBV infection, and/or decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In a particular embodiment, a method of treating HBV infection in a subject in need thereof, comprising administering to the subject a combination of LTα3 and IFNβ or a functional fragment thereof to decrease one or more symptoms of HBV infection in the subject, is provided. According to the said embodiment, the said combination for use in the treatment of an HBV infection, and/or decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In another aspect of the invention, a method of treating HBV infection in a subject in need thereof, comprising administering to the subject a combination of a tumor necrosis factor-like receptor weak inducer of apoptosis (TNFRSF12A) agonist and IFNβ or a functional fragment thereof to decrease one or more symptoms of HBV infection in the subject, is provided. According to the said aspect, the said combination for use in the treatment of an HBV infection, and/or decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In a particular embodiment, a method of treating HBV infection in a subject in need thereof, comprising administering to the subject a combination of TWEAK and IFNβ or a functional fragment thereof to decrease one or more symptoms of HBV infection in the subject, is provided. According to the said embodiment, the said combination for use in the treatment of an HBV infection, and/or decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In another aspect of the invention, a method of treating HBV infection in a subject in need thereof, comprising administering to the subject a combination of a LTβ receptor (TNFRSF3) agonist or a herpes virus entry mediator (HVEM or TNFRSF14) agonist and IFNβ or a functional fragment thereof to decrease one or more symptoms of HBV infection in the subject, is provided. According to the said aspect, the said combination for use in the treatment of an HBV infection, and/or decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In a particular embodiment, method of treating HBV infection in a subject in need thereof, comprising administering to the subject a combination of a LTβ receptor (TNFRSF3) agonist and IFNβ or a functional fragment thereof to decrease one or more symptoms of HBV infection in the subject, is provided. According to the said aspect, the said combination for use in the treatment of an HBV infection, and/or decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In a particular embodiment, method of treating HBV infection in a subject in need thereof, comprising administering to the subject a combination of a herpes virus entry mediator (HVEM or TNFRSF14) agonist and IFNβ or a functional fragment thereof to decrease one or more symptoms of HBV infection in the subject, is provided. According to the said aspect, the said combination for use in the treatment of an HBV infection, and/or decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In a particular embodiment, a method of treating HBV infection in a subject in need thereof, comprising administering to the subject a combination of LIGHT and IFNβ or a functional fragment thereof to decrease one or more symptoms of HBV infection in the subject, is provided. According to the said embodiment, the said combination for use in the treatment of an HBV infection, and/or decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In another aspect of the invention, a method of treating HBV infection in a subject in need thereof, comprising administering to the subject a combination of a LTα3 receptor agonist and IFNγ or a functional fragment thereof to decrease one or more symptoms of HBV infection in the subject, is provided. According to the said aspect, the said combination for use in the treatment of an HBV infection, and/or decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In a particular embodiment, a method of treating HBV infection in a subject in need thereof, comprising administering to the subject a combination of LTα3 and IFNγ or a functional fragment thereof to decrease one or more symptoms of HBV infection in the subject, is provided. According to the said embodiment, the said combination for use in the treatment of an HBV infection, and/or decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In another aspect of the invention, a method of treating HBV infection in a subject in need thereof, comprising administering to the subject a combination of a tumor necrosis factor-like receptor weak inducer of apoptosis (TNFRSF12A) agonist and IFNγ or a functional fragment thereof to decrease one or more symptoms of HBV infection in the subject, is provided. According to the said aspect, the said combination for use in the treatment of an HBV infection, and/or decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In a particular embodiment, a method of treating HBV infection in a subject in need thereof, comprising administering to the subject a combination of TWEAK and IFNγ or a functional fragment thereof to decrease one or more symptoms of HBV infection in the subject, is provided. According to the said embodiment, the said combination for use in the treatment of an HBV infection, and/or decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In another aspect of the invention, a method of treating HBV infection in a subject in need thereof, comprising administering to the subject a combination of a LTβ receptor (TNFRSF3) agonist or a herpes virus entry mediator (HVEM or TNFRSF14) agonist and IFNγ or a functional fragment thereof to decrease one or more symptoms of HBV infection in the subject, is provided. According to the said aspect, the said combination for use in the treatment of an HBV infection, and/or decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In a particular embodiment, a method of treating HBV infection in a subject in need thereof, comprising administering to the subject a combination of a LTβ receptor (TNFRSF3) agonist and IFNγ or a functional fragment thereof to decrease one or more symptoms of HBV infection in the subject, is provided. According to the said aspect, the said combination for use in the treatment of an HBV infection, and/or decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In a particular embodiment, a method of treating HBV infection in a subject in need thereof, comprising administering to the subject a combination of a herpes virus entry mediator (HVEM or TNFRSF14) agonist and IFNγ or a functional fragment thereof to decrease one or more symptoms of HBV infection in the subject, is provided. According to the said aspect, the said combination for use in the treatment of an HBV infection, and/or decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided

In a particular embodiment, a method of treating HBV infection in a subject in need thereof, comprising administering to the subject a combination of LIGHT and IFNγ or a functional fragment thereof to decrease one or more symptoms of HBV infection in the subject, provided. According to the said embodiment, the said combination for use in the treatment of an HBV infection, and/or decreasing one or more symptoms of HBV infection in the subject, is provided. Still according to the said embodiment, the use of the said combination for preparing a medicament for treating a HBV infection, and/or for decreasing one or more symptoms of HBV infection in the subject, is provided.

In another aspect of the invention, a method of treating HBV infection in a subject in need thereof, comprising administering to the subject a combination of Ig-TWEAK and IFNβ or a functional fragment thereof to decrease one or more symptoms of HBV infection in the subject, is provided. According to the said aspect, the said combination for use in the treatment of an HBV infection, and/or decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In another aspect of the invention, a method of treating HBV infection in a subject in need thereof, comprising administering to the subject a combination of Ig-CD40L and IFNβ or a functional fragment thereof to decrease one or more symptoms of HBV infection in the subject, is provided. According to the said aspect, the said combination for use in the treatment of an HBV infection, and/or decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In another aspect of the invention, a method of treating HBV infection in a subject in need thereof, comprising administering to the subject a combination of Ig-LTα3 and IFNβ or a functional fragment thereof to decrease one or more symptoms of HBV infection in the subject, is provided. According to the said aspect, the said combination for use in the treatment of an HBV infection, and/or decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In another aspect of the invention, a method of treating HBV infection in a subject in need thereof, comprising administering to the subject a combination of Ig-LIGHT and IFNβ or a functional fragment thereof to decrease one or more symptoms of HBV infection in the subject, is provided. According to the said aspect, the said combination for use in the treatment of an HBV infection, and/or decreasing one or more symptoms of HBV infection in the subject, is provided. Still according to the said aspect, the use of the said combination for preparing a medicament for treating a HBV infection, and/or for decreasing one or more symptoms of HBV infection in the subject, is provided.

In another aspect of the invention, a pharmaceutical composition comprising: a TNFRSF agonist; and a Type II IFN, a functional fragment of a Type II IFN, a Type III IFN, or a functional fragment of a Type III IFN, is provided.

In another aspect of the invention, a pharmaceutical composition comprising: a TNFRSF agonist; and a Type I IFN, or a functional fragment of a Type I IFN, is provided.

In another aspect of the invention, a pharmaceutical composition comprising: LTα3, LIGHT and/or TWEAK; and an IFN agent or a functional fragment thereof, is provided.

In another aspect of the invention, a pharmaceutical composition comprising: a TNFRSF agonist; and IFNα or a functional fragment thereof, is provided.

In another aspect of the invention, a pharmaceutical composition comprising: LTα3, LIGHT and/or TWEAK; and IFNα or a functional fragment thereof, is provided.

In another aspect of the invention, a pharmaceutical composition comprising: a CD40L; and IFNα or a functional fragment thereof, is provided.

In another aspect of the invention, a pharmaceutical composition comprising: a TNFRSF agonist; and IFNβ or a functional fragment thereof, is provided.

In another aspect of the invention, a pharmaceutical composition comprising: LTα3, LIGHT and/or TWEAK; and IFNβ or a functional fragment thereof, is provided.

In another aspect of the invention, a pharmaceutical composition comprising: a CD40L; and IFNβ or a functional fragment thereof, is provided.

In another aspect of the invention, a pharmaceutical composition comprising: a TNFRSF agonist; and IFNγ or a functional fragment thereof, is provided.

In another aspect of the invention, a pharmaceutical composition comprising: LTα3, LIGHT and/or TWEAK; and IFNγ or a functional fragment thereof, is provided.

In another aspect of the invention, a pharmaceutical composition comprising: a CD40L; and IFNγ or a functional fragment thereof, is provided.

In another aspect of the invention, a pharmaceutical composition comprising: a TNFRSF agonist; and IFNλ or a functional fragment thereof, is provided.

In another aspect of the invention, a pharmaceutical composition comprising: LTα3, LIGHT and/or TWEAK; and IFNλ or a functional fragment thereof, is provided.

In another aspect of the invention, a pharmaceutical composition comprising: a CD40L; and IFNλ or a functional fragment thereof, is provided.

### BRIEF DESCRIPTION OF THE DRAWINGS

The foregoing and other features and advantages of the present invention will be more fully understood from the following detailed description of illustrative embodiments taken in conjunction with the accompanying drawings.

FIG. 1A-FIG. 1B graphically depict the synergistic reduction of hepatitis B e-antigen (HBeAg) release using a combination of interferon beta (IFNβ) and cluster of differentiation 40 ligand (CD40L) or a combination of interferon alpha (IFNα) and cluster of differentiation 40 ligand (CD40L) in hepatitis B virus (HBV)-infected primary human hepatocytes. Primary human hepatocytes were assayed at 8 days post-infection, and were treated with IFNα, IFNβ and/or CD40L at day 2 and day 6. IFNα, 1000 U; IFNβ, 100 U; CD40L, 150 ng/mL. FIG. 1A schematically depicts the timeline of the experiment. FIG. 1B graphically shows levels of Hbe Ag with different agents.

FIG. 2A-FIG. 2B show the effect of IFNβ and CD40L on the transcription of the closed circular DNA (cccDNA) into pregenomic RNA (pgRNA) in HBV-infected primary hepatocytes. FIG. 2A schematically depicts the outline of the assay. FIG. 2B graphically shows that short-term stimulation was sufficient to greatly reduce pgRNA transcription.

FIG. 3A-FIG. 3B show the effects of IFNβ and CD40L on signaling pathways.

FIG. 3A graphically depicts CXCL10 release in the IFN pathway, demonstrating that a combination of IFNβ and CD40L synergistically enhance the IFN pathway. FIG. 3B graphically depicts IL8 release in the inflammatory pathway, demonstrating that IFNβ and CD40L do not synergistically enhance the inflammatory pathway.

FIG. 4A-FIG. 4B show the effects of different IFN types on CXCL10 release. FIG. 4A graphically depicts the effects of different TNF family members in combination with different IFN types. FIG. 4B graphically depicts the synergistic effect of costimulation of hepatocytes with CD40L and Type III interferons (IL28 and IL29).

FIG. 5 graphically depicts synergistic effects mediated by various CD40 agonists in combination with IFNβ on CXCL10 release. Specifically, FIG. 5 shows the effects of hexameric CD40L (hCD40L), trimeric CD40L (tCD40L) and anti-CD40 agonistic antibody (α-CD40) on CXCL10 release.

FIG. 6A-FIG. 6B graphically depict the potency of the synergistic effects of CD40L in combination with IFNβ. FIG. 6A shows the effects of CD40L in combination with IFNβ on CXCL10 release; Hash marked boxes represent saturated points. FIG. 6B shows the effect of CD40L in combination with IFNβ on IL8 release.

FIG. 7A-FIG. 7F graphically depict synergistic effects of CD40L in combination with IFNβ, as measured by transcriptomic analysis at select time points. It is expressed in Fragments Per Kilobase Of Exon Per Million Fragments Mapped (FPKM). FIG. 7A shows the effects of CD40L in combination with IFNβ on CXCL10 mRNA expression. FIG. 7B shows the effects of CD40L in combination with IFNβ on CXCL8/IL8 mRNA expression. FIG. 7C shows the effects of CD40L in combination with IFNβ on CXCL9 mRNA expression. FIG. 7D shows the effects of CD40L in combination with IFNβ on mRNA CXCL11 expression. FIG. 7E shows the effects of CD40L in combination with IFNβ on CXCL3 mRNA expression. FIG. 7F shows the effects of CD40L in combination with IFNβ on CCL20 mRNA expression.

FIG. 8 graphically depicts synergistic effects of CD40L in combination with IFNβ on CXCL10 release from primary human hepatocytes. CXCL10 release was assayed at day 3 and day 7 after stimulation of primary human hepatocytes (PHHs) infected or not by HBV.

FIG. 9A-FIG. 9B graphically depict synergistic effects of Ig-Tweak (FIG. 9A) and Ig-LIGHT (FIG. 9B) in combination with IFNβ on CXCL10 release in hepatocytes (HepaRG cells). FIG. 9C-FIG. 9D graphically depict the functional effect of Ig-Tweak (FIG. 9C) and Ig-LIGHT (FIG. 9D) on NFkB pathway in A549 cells. FIG. 9E-FIG. 9F graphically depict the boost of IFNb-induced antiviral effect by Ig-Tweak (FIG. 9E) and Ig-LIGHT (FIG. 9F) in combination with IFNb (Interferon beta) on primary hepatocytes infected by HBV. NS: Non stimulated; NT: Non treated.

FIG. 10A-FIG. 10B graphically depict the effect of three Duokine molecules on activating the CD40L-mediated NFκB pathway (FIG. 10A) and the Type I IFN-mediated JAK/STAT pathway reporter assay (FIG. 10B). The Duokines include an IFNβ-IgG1 Fc-CD40L fusion (IFNb-Ig-CD40L), an IFNβ-CD40L fusion with a simple linker (IFNb-CD40L), and an IFNβ-Leucine Zipper (LZ)-CD40L fusion (IFNb-LZ-CD40L).

FIG. 11A-FIG. 11B graphically depict the effect of the Duokine molecules on CXCL10 release from hepatocytes (hepaRG cells). FIG. 11A shows the effects of three different Duokine molecules alone on CXCL10 release. FIG. 11B shows the effect of a Duokine in combination with an anti-CD40L antagonistic antibody.

FIG. 12 graphically depicts the effect of Duokine molecules on CXCL10 release from hepatocytes (HepaRG cells). The Duokine is IFNβ-IgG1 Fc-Tweak fusion (IFNb-Ig-Tweak). Vvide: empty vector.

FIG. 13 graphically depicts the effect of Duokine molecules on CXCL10 release from hepatocytes. The Duokines include IFNβ-IgG1 Fc-Light fusion (IFNb-Ig-Light), and an IFNβ-Leucine Zipper (LZ)-Light fusion (IFNb-LZ-Light). Vvide: empty vector.

FIG. 14A-FIG. 14B graphically depict persistence of viremia and viral antigens at various time points up to 49 days post-injection of an adeno-associated virus (AAV)/HBV in mice. FIG. 14A shows serum HBV DNA levels. FIG. 14B shows serum HBV antigen levels.

FIG. 15 depicts cccDNA formation in an AAV/HBV model.

FIG. 16 graphically depicts the synergistic effects of mCD40L in combination with mIFNβ on CXCL10 release in vivo murine model. “MIX-mCD40L”: mIFNb and mCD40L combination.

FIG. 17A graphically depicts the activity of recombinant murine mIFNb-Fc-mIgG1 on the interferon pathway using IFN-reporter raw-dual cells. FIG. 17B graphically depicts the activity of recombinant murine Fc-mIgG1-mCD40L on CD40-induced NFkB pathway in HEK-CD40 reporter cells. FIG. 17C graphically depicts the ELISA dosage of mIFNb-Fc-mIgG1 in the serum of mice administrated with 0,84 μg of the recombinant protein. FIG. 17D graphically depicts the dosage of Fc-mIgG1-mCD40L in the serum sampled at different times after administration of mice with 30 μg of the recombinant protein.

FIG. 18 depicts antiviral activities of a murine fusion protein comprising interferon beta (mIFNb-Fc-mIgG1) and a murine fusion protein comprising CD40L (Fc-mIgG1-mCD40L) alone or in combination in the AAV/HBV-transduced mouse model. Shown are study design and study groups (FIG. 18A) and viral parameters at each sampling time. Peripheral blood viral protein HBe-Ag (FIG. 18B), viremia: peripheral blood HBV DNA level (FIG. 18C) and liver HBV DNA level (FIG. 18D) are expressed as individual values with mean +/− sem. Liver HBV pgRNA (FIG. 18E) is expressed as fold change individual data with Geometric mean.

FIG. 19A-FIG. 19C graphically depict the functional activity of the fusion protein IFNa-huIgG1-huCD40L on HEK-Blue-CD40 cells (FIG. 19A), HEK-Blue-IFNa/b cells (FIG. 19B) and on HBV infection (Hbe release) of primary hepatocytes (FIG. 19C). IFNa: Interferon alpha.

### DETAILED DESCRIPTION

The present invention is based in part on the discovery of a combination therapy that synergistically inhibits transcription of hepatitis B virus (HBV) covalently closed circular DNA (cccDNA) into pregenomic RNA (pgRNA) in HBV-infected cells, synergistically inhibits release of hepatitis B e-antigen (HBeAg) from HBV-infected cells, and synergistically enhances the IFN pathway in uninfected and HBV infected hepatocytes, in particular in uninfected and HBV infected primary human hepatocytes. Combination therapies comprising administering a TNFRSF agonist (e.g., an agonistic antibody directed against a member of the TNFRSF, a soluble TNFRSF agonist including but not limited to its natural ligand) or a functional fragment thereof and an interferon (IFN) agent or a functional fragment thereof to an HBV-infected cell, or subject infected with HBV, are provided.

The disclosure may be more readily understood in the light of the selected terms defined below.

As used herein, a “TNFRSF agonist” refers to a compound (e.g., a protein, a fusion protein, a polypeptide, an antibody, an antigen-binding fragment of an antibody or the like) that activates a TNFRSF. For example, a TNFRSF agonist may be an agonistic antibody directed against a member of the TNFRSF, a soluble TNFRSF agonist including but not limited to its natural ligand or a functional fragment of thereof.

As used herein, the term “antibody” refers to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated VH or V_{H}) and a heavy chain constant region (CH or C_{H}). The heavy chain constant region comprises three domains, CH1, CH2 and CH3. Each light chain comprises a light chain variable region (abbreviated VL) and a light chain constant region (CL or C_{L}). The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

As used herein, the term “ligand” refers to any substance capable of binding, or of being bound, to another substance. A ligand may be a peptide, a polypeptide, a protein, an aptamer, a polysaccharide, a sugar molecule, a carbohydrate, a lipid, an oligonucleotide, a polynucleotide, a synthetic molecule, an inorganic molecule, an organic molecule, and any combination thereof.

As used herein, the term “functional fragment” refers to a fragment of a substance that retains one or more functional activities of the original substance. For example, a functional fragment of a TNFRSF agonist refers to a fragment of a TNFRSF agonist that retains a function of the TNFRSF agonist as described herein, e.g., it activates a target TNFRSF. A functional fragment of an interferon refers to a fragment of an interferon that retains an IFN function as described herein, e.g., it mediates IFN pathway signaling.

As used herein, “hepatitis B virus” or “HBV” refers to the double stranded DNA virus that causes hepatitis B, which belongs to a family of closely related DNA viruses called the Hepadnaviruses. Hepadnaviruses have a strong preference for infecting liver cells, but small amounts of hepadnaviral DNA can be found in kidney, pancreas, and mononuclear cells. However, infection at these sites is not linked to extra hepatic disease.

The HBV virion, i.e., the Dane particle, consists of an outer lipid envelope and an icosahedral nucleocapsid core composed of protein. The nucleocapsid encloses the viral DNA and a DNA polymerase that has reverse transcriptase activity similar to retroviruses. The outer envelope contains embedded proteins which are involved in viral binding of, and entry into, susceptible cells. The virus is one of the smallest enveloped animal viruses with a virion diameter of 42 nm, but pleomorphic forms exist, including filamentous and spherical bodies lacking a core. These particles are not infectious and are composed of the lipid and protein that forms part of the surface of the virion, which is called the surface antigen (HBsAg), and is produced in excess during the life cycle of the virus. HBV comprises HBsAg, HBcAg (and its splice variant HBeAg), DNA polymerase and Hbx. HBV is one of a few known non-retroviral viruses which employ reverse transcription as a part of its replication process.

The HBV nucleocapsid contains a relatively small and partially duplex 3.2 kb circular DNA, viral polymerase and core protein. The genome has only four long open reading frames. The pre-S-S (pre-surface-surface) region of the genome encodes the three viral surface antigens by differential initiation of translation at each of three in-frame initiation codons.

The most abundant protein of HBV is the 24 kD S protein (which is known as HBsAg). The pre-C-C (pre-core-core) region encodes HBcAg (HBV core Antigen) and HBeAg (HBV e Antigen). HBeAg is not required for viral replication and plays no role in viral assembly. The P-coding region is specific for the viral polymerase, a multifunctional enzyme involved in DNA synthesis and RNA encapsidation. The X open reading frame encodes the viral X protein (HBx), which modulates host-cell signal transduction and can directly and indirectly affect host and viral gene expression.

The life cycle of HBV is believed to begin when the virus attaches to the host cell membrane via its envelope proteins. It has been suggested that HBV binds to a receptor on the plasma membrane that is predominantly expressed on human hepatocytes via the pre-S1 domain of the large envelope protein as an initial step in HBV infection. However, the nature of the receptor remains controversial. Then, the viral membrane fuses with the cell membrane and the viral genome is released into the cells.

Replication of HBV can be regulated by a variety of factors, including hormones, growth factors, and cytokines. After the viral genome reaches the nucleus, the viral polymerase converts the partial double-stranded DNA (dsDNA) genome into covalently closed circular DNA (cccDNA). This DNA is transcribed by host RNA Pol-II, and the resulting DNA is the template for further propagation of pre-genomic RNA and sub-genomic RNA.

The pre-genomic RNA is bifunctional, serving as both the template for viral DNA synthesis and as the messenger for pre-C, C, and P translation. The sub-genomic RNAs function exclusively for translation of the envelope and X protein. All viral RNA is transported to the cytoplasm, where its translation yields the viral envelope, core, and polymerase proteins, as well as HBx and HBcAg.

HBV core particles are assembled in the cytosol and during this process a single molecule of pre-genomic RNA is incorporated into the assembling viral core. Once the viral RNA is encapsidated, reverse transcription begins. The synthesis of the two viral DNA strands is sequential. The first DNA strand is made from the encapsidated RNA template; during or after the synthesis of this strand, the RNA template is degraded and the synthesis of the second DNA strand proceeds, with the use of the newly made first DNA strand as a template. Some cores bearing the mature genome are transported back to the nucleus, where their newly minted DNA genomes can be converted to cccDNA to maintain a stable intranuclear pool of transcriptional templates.

HBV surface antigen (HBsAg) proteins are initially synthesized and polymerized in the rough endoplasmic reticulum. These proteins are transported to the post-ER and pre-Golgi compartments, where budding of the nucleocapsid follows. The assembled HBV virion and sub-viral particles are transported to the Golgi for further modification of glycans of the surface proteins, and then are secreted out of the host cell to finish the life cycle.

In particular embodiments, the methods and compositions described herein can be used to synergistically inhibit release of HBeAg from HBV-infected cells, and/or synergistically enhance the IFN pathway in HBV-infected cells (e.g., hepatocytes). As used herein, the terms “synergistically” and “synergistic” refer to an effect that is mediated by two or more components (e.g., a TNFRSF agonist and an IFN or functional fragment thereof) that is greater than the added effect of each component used separately. A synergistic effect can be about 5%, 10%, 15%, 20%, 25%, 30%, 35%, 40%, 45%, 50%, 55%, 60%, 65%, 70%, 75%, 80%, 85%, 90%, 95%, or 100% or greater than the added effect of the components individually.

In particular embodiments, the methods and compositions described herein can be used to treat HBV infection. As used herein, “treat HBV infection” and “treatment of HBV infection” refers to one or more of: (i) reducing HBV viral load/viral titer (i.e., reducing the number of infectious viral particles per mL); (ii) reducing the transcription of cccDNA; (iii) reducing the level of pre-genomic RNA in cells; (iv) decreasing one or more HBV-related disorders; and (v) decreasing one or more HBV-related symptoms in a subject.

In particular embodiments, the methods and compositions described herein can be used to reduce the HBV viral load/viral titer of an HBV-infected cell. HBV viral load/viral titer may be reduced by about 5%, 10%, 15%, 20%, 25%, 30%, 35%, 40%, 45%, 50%, 55%, 60%, 65%, 70%, 75%, 80%, 85%, 90%, 95%, or 100% compared to an untreated HBV-infected cell.

In particular embodiments, the methods and compositions described herein can be used to reduce transcription of HBV cccDNA in an HBV-infected cell. cccDNA transcription may be reduced by about 5%, 10%, 15%, 20%, 25%, 30%, 35%, 40%, 45%, 50%, 55%, 60%, 65%, 70%, 75%, 80%, 85%, 90%, 95%, or 100% compared to an untreated HBV-infected cell.

In particular embodiments, the methods and compositions described herein can be used to reduce the level of pre-genomic HBV RNA in an HBV-infected cell. Pre-genomic HBV RNA levels may be reduced by about 5%, 10%, 15%, 20%, 25%, 30%, 35%, 40%, 45%, 50%, 55%, 60%, 65%, 70%, 75%, 80%, 85%, 90%, 95%, or 100% compared to an untreated HBV-infected cell.

As used herein, an “HBV-related disorder” refers to a disorder that results from infection of a subject by HBV. HBV-related disorders include, but are not limited to acute hepatitis, chronic hepatitis, icteric hepatitis, fulminant hepatitis, sub-fulminant hepatitis, and symptoms and/or complications arising from any of these disorders.

As used herein, an “HBV-related symptom,” a “symptom of HBV infection” or an “HBV-related complication” includes one or more physical dysfunctions related to HBV infection. HBV symptoms and complications include, but are not limited to, cirrhosis, hepatocellular carcinoma (HCC), membranous glomerulonephritis (MGN), death, acute necrotizing vasculitis (polyarteritis nodosa), membranous glomerulonephritis, papular acrodermatitis of childhood (Gianotti-Crosti syndrome), HBV-associated nephropathy (e.g., membranous glomerulonephritis), immune-mediated hematological disorders (e.g., essential mixed cryoglobulinemia, aplastic anemia), portal hypertension, ascites, encephalopathy, jaundice, pruritus, pale stools, steatorrhea, polyarteritis nodosa, glomerular disease, abnormal ALT levels, abnormal AST levels, abnormal alkaline phosphatase levels, increased bilirubin levels, anorexia, malaise, fever, nausea, vomiting and the like.

As used herein, an “interferon agent” or “IFN” refers to a cytokine, or derivative thereof, that is typically produced and released by cells in response to the presence of a pathogen or a tumor cell. IFNs include type I IFNs (e.g., IFNα, IFNβ, IFNε, IFNκ, IFNτ, IFNζ and IFNω), type II IFNs (e.g., IFNγ) and type III IFNs (e.g., IFNλ1, IFNλ2 and IFNλ3).

According to certain exemplary embodiments, a combination therapy described herein utilizes one or more of a full-length IFN, a modified variant thereof (e.g., a chemically (e.g., PEGylated) modified variant or mutein), or a biologically active fragment thereof, that retains one or more signaling activities of a full-length IFN. In certain embodiments, the IFN agent is a human IFN agent.

In certain embodiments, a combination therapy described herein utilizes one or more of an IFNα, an IFNα fragment, an IFNβ, an IFNβ fragment, an IFNγ, an IFNγ fragment, an IFNλ, or an IFNλ fragment.

In other exemplary embodiments a combination therapy described herein utilizes one or more of an IFNα, an IFNα fragment, an IFNβ, an IFNβ fragment, an IFNγ, an IFNγ fragment, an IFNλ, or an IFNλ fragment that is part of a fusion protein such as, e.g., a bifunctional immunostimulatory fusion protein (e.g., a TNFRSF agonist/IFN Duokine). In still other exemplary embodiments, a combination therapy described herein utilizes one or more of an IFNα, an IFNα fragment, an IFNβ, an IFNβ fragment, an IFNγ, an IFNγ fragment, an IFNλ, or an IFNλ fragment that is expressed by a nucleic acid sequence.

In certain embodiments, the expression level of one or more IFN signaling pathway biomarkers is altered, i.e., upregulated or downregulated, in an HBV-infected cell treated with a combination therapy described herein (e.g., a combination of a TNFRSF agonist and an IFN agent or fragment thereof). According to certain exemplary embodiments, the expression level of one or more IFN pathway biomarkers is upregulated in an HBV-infected cell treated with a combination therapy described herein (e.g., a combination of a TNFRSF agonist or fragment thereof and an IFN or fragment thereof).

According to certain embodiments, a suitable IFN pathway biomarker featured herein is a chemokine, e.g., a C-X-C chemokine, selected from the group consisting of CXCL9, CXCL10 and CXCL11. In certain exemplary embodiments, a suitable biomarker induced by the IFN pathway is CXCL9, CXCL10 and/or CXCL11 and also the interferon stimulated gene ISG20.

As used herein, a tumor necrosis factor (ligand) superfamily member (or TNFSF) refers to a protein belonging to a superfamily of protein ligands that share a hallmark extracellular TNF homology domain (THD) (Bremer ISRN Oncology (2013), Article ID 371854, 25 pages, online access: dx.doi.org/10.1155/2013/371854). The THD triggers formation of non-covalent homotrimers. TNF ligands are typically expressed as type II transmembrane proteins, but most can be subject to proteolytic processing into a soluble ligand. TNF ligands exert their biological function by binding to and activating members of the TNFRSF. TNFRSFs are typically expressed as trimeric type I transmembrane proteins and contain one to six cysteine-rich domains (CRDs) in their extracellular domain. An important function of the TNF superfamily is the provision of co-stimulatory signals at distinct stages of an immune response. Some ligands have the capacity to bind and activate different receptors (e.g., LTα3 which binds and activates TNFRSF1A, TNFRSF1B and TNFRSF14 and LIGHT (TNFSF14) which binds and activates TNFRSF3 and TNFRSF14). Exemplary TNFSF gene family members are recited below in Table 1, derived from the HUGO Gene Nomenclature Committee (HGNC) (see, Gray et al. Nucleic Acids Res. 43: D1079-1085 (2015); HGNC Database, HUGO Gene Nomenclature Committee (HGNC), EMBL Outstation-Hinxton, European Bioinformatics Institute, Wellcome Trust Genome Campus, Hinxton, Cambridgeshire, CB10 1SD, UK www.genenames.org). The Approved Symbol denotes the HGNC symbol applied to a particular gene and the Approved Name corresponds to the full spelling of the gene. Previous Symbols denotes any previous symbol used by HGNC to refer to a particular gene. Synonyms refer to alternative, synonymous names for a particular gene.

TABLE 1
Exemplary TNFSF gene family members.

| Approved Symbol | Approved Name | Previous Symbols | Synonyms |
|---|---|---|---|
| TNF | tumor necrosis factor | TNFA | TNFSF2, DIF, TNFα |
| LTA | lymphotoxin alpha | TNFB | LT, TNFSF1 |
| LTB | lymphotoxin beta | TNFC | TNFSF3, p33 |
| TNFSF4 | TNF superfamily member 4 | TXGP1 | OX-40L, gp34, CD252 |
| CD40LG | CD40 ligand | TNFSF5, HIGM1, IMD3 | CD40L, TRAP, gp39, hCD40L, CD154 |
| FASLG | Fas ligand | TNFSF6, APT1LG1 | FasL, CD178 |
| CD70 | CD70 molecule | TNFSF7, CD27L | CD27L |
| TNFSF8 | TNF superfamily member 8 | | CD153 |
| TNFSF9 | TNF superfamily member 9 | | 4-1BB-L |
| TNFSF10 | TNF superfamily member 10 | | TRAIL, Apo-2L, TL2, CD253 |
| TNFSF11 | TNF superfamily member 11 | | TRANCE, RANKL, OPGL, ODF, CD254 |
| TNFSF12 | TNF superfamily member 12 | | TWEAK, DR3LG, APO3L |
| TNFSF13 | TNF superfamily member 13 | | APRIL, CD256 |
| TNFSF13B | TNF superfamily member 13B | TNFSF20 | BAFF, THANK, BLYS, TALL-1, |
| | | | TALL1, CD257 |
| TNFSF14 | TNF superfamily member 14 | | LIGHT, LTγ, HVEM-L, CD258 |
| TNFSF15 | TNF superfamily member 15 | | TL1, VEGI, TL1A, VEGI192A, |
| | | | MGC129934, MGC129935 |
| TNFSF18 | TNF superfamily member 18 | | AITRL, TL6, hGITRL |
| EDA | ectodysplasin A | ED1, EDA2, ODT1 | EDA1, XLHED, HED, XHED, |
| | | | ED1-A1, ED1-A2, EDA-A1, EDA-A2 |

TABLE 2
Exemplary TNFRSF gene family members.

| Approved Symbol | Approved Name | Previous Symbols | Synonyms |
|---|---|---|---|
| EDAR | ectodysplasin A receptor | ED3, DL | ED5, EDA3, Edar, ED1R, EDA1R |
| TNFRSF1A | TNF receptor | TNFR1 | TNF-R, TNFAR, TNFR60, TNF-R-I, |
| | superfamily member 1A | | CD120a, TNF-R55 |
| TNFRSF1B | TNF receptor | TNFR2 | TNFBR, TNFR80, TNF-R75, |
| | superfamily member 1B | | TNF-R-II, p75, CD120b |
| LBTR | Lymphotoxin beta receptor | D12S370 | TNF-R-III, TNFCR, TNFRSF3, |
| | | | TNFR2-RP, TNFR-RP |
| TNFRSF4 | TNF receptor | TXGP1L | ACT35, OX40, CD134 |
| | superfamily member 4 | | |
| CD40 | CD40 molecule | TNFRSF5 | p50, Bp50 |
| FAS | Fas cell surface | APT1, FAS1, TNFRSF6 | CD95, APO-1 |
| | death receptor | | |
| TNFRSF6b | TNF receptor | | DcR3, DCR3, TR6, M68 |
| | superfamily member 6b | | |
| CD27 | CD27 molecule | TNFRSF7 | S152, Tp55 |
| TNFRSF8 | TNF receptor | CD30, D1S166E | KI-1 |
| | superfamily member 8 | | |
| TNFRSF9 | TNF receptor | ILA | CD137, 4-1BB |
| | superfamily member 9 | | |
| TNFRSF10a | TNF receptor | | DR4, Apo2, TRAILR-1, CD261, |
| | superfamily member 10a | | TRAILR1 |
| TNFRSF10b | TNF receptor | | DR5, KILLER, TRICK2A, |
| | superfamily member 10b | | TRAIL-R2, TRICKB, CD262, TRAILR2 |
| TNFRSF10c | TNF receptor | | DcR1, TRAILR3, LIT, TRID, |
| | superfamily member 10c | | CD263 |
| TNFRSF10d | TNF receptor | | DcR2, TRUNDD, TRAILR4, CD264 |
| | superfamily member 10d | | |
| TNFRSF11a | TNF receptor | PDB2, LOH18CR1 | RANK, CD265, FEO |
| | superfamily member 11a | | |
| TNFRSF11b | TNF receptor | OPG | OCIF, TR1 |
| | superfamily member 11b | | |
| TNFRSF12A | TNF receptor | | FN14, TweakR, CD266 |
| | superfamily member 12A | | |
| TNFRSF13B | TNF receptor | | TAC1, CD267, IGAD2 |
| | superfamily member 13B | | |
| TNFRSF13C | TNF receptor | | BAFFR, CD268 |
| | superfamily member 13C | | |
| TNFRSF14 | TNF receptor | | HVEM, ATAR, TR2, LIGHTR, |
| | superfamily member 14 | | HVEA, CD270 |
| NGFR | Nerve grwoth factor receptor | | TNFRSF16, p75NTR, CD271 |
| TNFRSF17 | TNF receptor | BCMA | BCM, CD269, TNFRSF13A |
| | superfamily member 17 | | |
| TNFRSF18 | TNF receptor | | AFTR, GITR, CD357 |
| | superfamily member 18 | | |
| TNFRSF19 | TNF receptor | | TAJ-alpha, TROY, TAJ, TRADE |
| | superfamily member 19 | | |
| RELT | RELT, TNF receptor | TNFRSF19L | FLJ14993 |
| TNFRSF21 | TNF receptor | | DR6, CD358 |
| | superfamily member 21 | | |
| TNFRSF25 | TNF receptor | TNFRSF12 | DR3, TRAMP, WSL-1, LARD, |
| | superfamily member 25 | | WSL-LR, DDR3, TR3, APO-3 |
| EDA2R | Ectodysplasin A2 receptor | | XEDAR, EDAA2R, EDA-A2R, TNFRSF27 |

As used herein, a “TNFRSF agonist” refers to a compound (e.g., a protein, a fusion protein, a polypeptide, an antibody, an antigen-binding fragment of an antibody or the like) that activates a TNFRSF, e.g., a TNFRSF listed in Table 2. Table 2 is derived from the HGNC, as for Table 1 above. For example, a TNFRSF agonist may be an agonistic antibody directed against a member of the TNFRSF, a soluble TNFRSF agonist including but not limited to its natural ligand or a functional fragment of thereof.

In certain exemplary embodiments, a TNFRSF agonist includes, but is not limited to, a LTα3 receptors (TNFRSF1A, TNFRSF1B, or TNFRSF14) agonist, a LTI3 receptor (TNFRSF3) agonist (e.g., LIGHT or LTβ), a herpesvirus entry mediator (HVEM or TNFRSF14) agonist (LIGHT), a tumor necrosis factor-like receptor weak inducer of apoptosis (TNFRSF12A) agonist (e.g., TWEAK also known as TNFSF12), a cluster of differentiation factor 40 (CD40, TNFRSF5) agonist (CD40L), a CD27 (TNFRSF7) agonist (CD70), a CD30 (TNFRSF8) agonist, a 4-1BB (CD137, TNFRSF9) agonist, a receptor activator of nuclear factor κB (RANK, TNFRSF11A) agonist, a Troy (TNFRSF19) agonist, and an OX40 receptor (TNFRSF4) agonist.

According to certain exemplary embodiments, a combination therapy described herein utilizes one or more of an agonistic antibody directed against a TNF receptor, a soluble TNFRSF agonist including but not limited to its natural ligand, or modified variant (e.g., mutein) or biologically active fragment of either, that retains one or more signaling activities of a full-length or soluble TNFRSF agonist.

In certain embodiments, a combination therapy described herein utilizes one or more TNFRSF agonists. According to certain exemplary embodiments, a TNFRSF agonist is provided as a multimer (e.g., a dimer, a trimer, a tetramer, pentamer, a hexamer, a heptamer, an octamer or the like) or as a fusion protein, e.g., a fusion protein comprising a TNFRSF agonist and an IFN agent or a biologically active fragment of an IFN agent (See, e.g., Table 3 for examples of suitable fusion proteins). In other embodiments, a TNFRSF agonist is provided as an agonistic antibody or antigen-binding fragment thereof.

In certain embodiments, a combination therapy described herein utilizes one or more TNFRSF agonists selected from CD40L, TWEAK, LIGHT and LTα3. In certain exemplary embodiments, a combination therapy described herein utilizes one or more CD40 agonists that is a multimeric CD40L, e.g., a hexameric CD40L, a trimeric CD40L or the like. In other exemplary embodiments, a combination therapy described herein utilizes one or more anti-CD40 agonistic antibodies. Examples of suitable anti-CD40 agonistic antibodies include, but are not limited to, CP-870,893 (Pfizer/Roche), SGN-40 (Seattle Genetics), ADC-1013 (Janssen/Alligator BioSciences), Chi Lob 7/4 (University of Southampton), dacetuzmumab (Seattle Genetics), APX005M (Apexigen, Inc.), 3G5 (Celldex) and CDX-1140 (Celldex). In still other exemplary embodiments, a combination therapy described herein utilizes one or more CD40 agonists such as CD40L/IFNb fusion proteins, e.g., bifunctional immunostimulatory fusion proteins (e.g., CD40L/IFN Duokines (See, e.g., WO 2016/113395, incorporated herein by reference in its entirety for all purposes)). In yet other exemplary embodiments, a combination therapy described herein utilizes one or more CD40 agonists expressed by a nucleic acid sequence. The term “fusion protein,” as used herein, generally refers to a protein created by joining two or more distinct peptides or proteins, resulting in a single protein with one or more functional properties derived from each of the original proteins. A fusion protein encompasses monomeric and multimeric, e.g., dimeric, trimeric, tetrameric or the like, complexes of distinct fusion proteins.

In certain exemplary embodiments, a fusion protein has the general formula:

Aₙ−L−Bₘ (Formula I)

wherein A is a TNFRSF agonist or a functional fragment thereof, B is an IFN agent or a functional fragment thereof, and L comprises, or alternatively consists of, a linker. In certain embodiments, two or more fusion proteins of Formula I may be linked to one another via one or more additional linkers “L”.

“n,” as used in An, refers to 1, 2, 3, 4, 5, 6, 7, 8 or more TNFRSF agonists or functional fragments thereof that may be associated with one or more linkers “L.” In certain embodiments, two or more TNFRSF agonists or functional fragments thereof are associated with a single linker. In other embodiments, two or more TNFRSF agonists or functional fragments thereof are complexed with one another (e.g., as an oligomer), and one or more of the complexed TNFRSF agonists or functional fragments thereof are associated with one or more linkers. In still other embodiments, an individual TNFRSF agonist or functional fragment thereof is associated with a single linker.

“m,” as used in Bₘ, refers to 1, 2, 3, 4, 5, 6, 7, 8 or more IFN agents or functional fragments thereof, wherein “m” may be associated with two or more ligands or fragments or variants thereof that may be associated with one or more linkers “L.” In certain embodiments, two or more ligands or fragments or variants thereof are associated with a single linker. In other embodiments, two or more ligands or fragments or variants thereof are complexed with one another (e.g., as an oligomer), and one or more of the complexed ligands or fragments or variants thereof are associated with one or more linkers. In still other embodiments, an individual ligand or fragment or variant thereof is associated with a single linker.

The term “linker” or “L,” as used herein, refers to any moiety that covalently joins one or more Aₙ to one or more Bₘ. In exemplary embodiments, a linker is a peptide linker. The term “peptide linker,” as used herein, refers to a peptide adapted to link two or more ligands or fragments or variants thereof. A peptide linker may have any length, i.e., comprise any number of amino acid residues. A linker is typically long enough to provide an adequate degree of flexibility to prevent the linked moieties from interfering with each other's activity, e.g., the ability of a ligand to multimerize and/or bind to a receptor. In exemplary embodiments, the linker is or comprises an Fc domain (e.g., a human IgG1 or IgG3 Fc domain) or fragment thereof. In other embodiments, the linker is Gly-Ser or a Gly-Ser-Thr linker composed of multiple glycine, serine and, where applicable, threonine residues. In other embodiments, the linker is a combination of an Fc domain or a fragment thereof and a Gly-Ser linker or a Gly-Ser-Thr linker. The sequences of peptide linker according to certain exemplary embodiments are set forth in Table 3.

L may further comprise a multimerization domain allowing the multimerization of the fusion protein. In such cases, L may comprise a peptide linker, in which the multimerization domain has been inserted. In an alternative embodiment, L may comprise two peptide linkers, wherein the two peptide linkers may be the same or different. In yet another embodiment, a multimerization domain represents the peptide linker comprised by L.

Multimerization may occur by non-covalent interaction and/or covalent interaction, in particular via one or more disulfide bonds or by aligning multiple coding sequences of the same molecule, between multiple (e.g., 2, 3, 4 or more) multimerization domains.

Suitable multimerization domains are known to a person skilled in the art and include, for example, a tenascin trimerization motif, a collectin trimerization domain and streptavidin, and dimerization domains, such as an IgE heavy-chain domain 2 (EHD2), an IgM heavy-chain domain 2 (MHD2), an IgG heavy-chain domain 3 (GHD3), an IgA heavy-chain domain 3 (AHD2), an IgD heavy-chain domain 3 (DHD3), an IgE heavy-chain domain 4 (EHD4), an IgM heavy-chain domain 4 (MHD4), an Fc domain, an uteroglobin dimerization domain and a leucine zipper (LZ) domain.

In a particular embodiment, a fusion protein may be a bifunctional immunostimulatory fusion protein comprising the TNFRSF agonist or a functional fragment thereof, the IFN agent or a functional fragment thereof and a linker.

In an embodiment of any one of the aspects of the invention, a bifunctional immunostimulatory fusion protein is a protein comprising a TNFRSF agonist or a functional fragment thereof, an IFN agent or a functional fragment thereof and a linker.

In an embodiment of any one of the aspects of the invention, a bifunctional immunostimulatory fusion protein is a protein comprising CD40L or a functional fragment thereof, an IFNβ or a functional fragment thereof and a linker.

In an embodiment of any one of the aspects of the invention, a bifunctional immunostimulatory fusion protein is a protein comprising CD40L or a functional fragment thereof, an IFNβ or a functional fragment thereof and a linker.

In an embodiment of any one of the aspects of the invention, a bifunctional immunostimulatory fusion protein is a protein comprising CD40L or a functional fragment thereof, an IFNγ or a functional fragment thereof and a linker.

In an embodiment of any one of the aspects of the invention, a bifunctional immunostimulatory fusion protein is a protein comprising CD40L or a functional fragment thereof, an IFNλ or a functional fragment thereof and a linker.

In an embodiment of any one of the aspects of the invention, a bifunctional immunostimulatory fusion protein is a protein comprising TWEAK or a functional fragment thereof, an IFNβ or a functional fragment thereof and a linker.

In an embodiment of any one of the aspects of the invention, a bifunctional immunostimulatory fusion protein is a protein comprising TWEAK or a functional fragment thereof, an IFNβ or a functional fragment thereof and a linker.

In an embodiment of any one of the aspects of the invention, a bifunctional immunostimulatory fusion protein is a protein comprising TWEAK or a functional fragment thereof, an IFNγ or a functional fragment thereof and a linker.

In an embodiment of any one of the aspects of the invention, a bifunctional immunostimulatory fusion protein is a protein comprising TWEAK or a functional fragment thereof, an IFNλ or a functional fragment thereof and a linker.

In an embodiment of any one of the aspects of the invention, a bifunctional immunostimulatory fusion protein is a protein comprising LIGHT or a functional fragment thereof, an IFNβ or a functional fragment thereof and a linker.

In an embodiment of any one of the aspects of the invention, a bifunctional immunostimulatory fusion protein is a protein comprising LIGHT or a functional fragment thereof, an IFNβ or a functional fragment thereof and a linker.

In an embodiment of any one of the aspects of the invention, a bifunctional immunostimulatory fusion protein is a protein comprising LIGHT or a functional fragment thereof, an IFNγ or a functional fragment thereof and a linker.

In an embodiment of any one of the aspects of the invention, a bifunctional immunostimulatory fusion protein is a protein comprising LIGHT or a functional fragment thereof, an IFNλ or a functional fragment thereof and a linker.

As used herein, a “Duokine” refers to a fusion protein comprising one, two or more cytokines. The sequences of Duokines and components thereof according to certain exemplary embodiments are set forth in Table 3.

TABLE 3
Sequences of exemplary Duokine molecules and
components thereof. Bold sequences correspond to linkers and italic sequences
correspond to signal peptides.

| Duokine/Components | |
|---|---|
| thereof | Sequence |
| IFNβ | MTNKCLLQIALLLCFSTTALSMSYNLLGFLQRSSNFQCQK |
| (SEQ ID NO: 1) | LLWQLNGRLEYCLKDRMNFDIPEEIKQLQQFQKEDAAL |
| | TIYEMLQNIFAIFRQDSSSTGWNETIVENLLANVYHQIN |
| | HLKTVLEEKLEKEDFTRGKLMSSLHLKRYYGRILHYLK |
| | AKEYSHCAWTIVRVEILRNFYFINRLTGYLRN |
| CD40L soluble form | MQKGDQNPQIAAHVISEASSKTTSVLQWAEKGYYTMS |
| (SEQ ID NO: 2) | NNLVTLENGKQLTVKRQGLYYIYAQVTFCSNREASSQA |
| | PFIASLCLKSPGRFERILLRAANTHSSAKPCGQQSIHLGG |
| | VFELQPGASVFVNVTDPSQVSHGTGFTSFGLLKL |
| TWEAK soluble form | MKGRKTRARRAIAAHYEVHPRPGQDGAQAGVDGTVSG |
| (SEQ ID NO: 3) | WEEARINSSSPLRYNRQIGEFIVTRAGLYYLYCQVHFDE |
| | GKAVYLKLDLLVDGVLALRCLEEFSATAASSLGPQLRL |
| | CQVSGLLALRPGSSLRIRTLPWAHLKAAPFLTYFGLFQV |
| | H |
| LIGHT soluble form | DGPAGSWEQLIQERRSHEVNPAAHLTGANSSLTGSGGP |
| (SEQ ID NO: 4) | LLWETQLGLAFLRGLSYHDGALVVTKAGYYYIYSKVQ |
| | LGGVGCPLGLASTITHGLYKRTPRYPEELELLVSQQSPC |
| | GRATSSSRVWWDSSFLGGVVHLEAGEKVVVRVLDERL |
| | VRLRDGTRSYFGAFMV |
| Peptide linker IgG1 Fc | DDKTHTCPPCPAPELLGGPSVFLFPPKPKDTLMISRTPEV |
| Domain | TCVVVDVSHEDPEVKFNWYVDGVEVHNAKTKPREEQY |
| (SEQ ID NO: 5) | NSTYRVVSVLTVLHQDWLNGKEYKCKVSNKALPAPIE |
| | KTISKAKGQPREPQVYTLPPSRDELTKNQVSLTCLVKGF |
| | YPSDIAVEWESNGQPENNYKTTPPVLDSDGSFFLYSKLT |
| | VDKSRWQQGNVFSCSVMHEALHNHYTQKSLSLSPGK |
| Peptide Gly-Ser-Thr | |
| linker | |
| (SEQ ID NO: 6) | SGGTSGSTSGTGS |
| (SEQ ID NO: 7) | SGGTSGSTSGTGST |
| (SEQ ID NO: 27) | GGGGSGGGGSGGGGS |
| (SEQ ID NO: 28) | GSPAPDPAPDPSG |
| Peptide linker | VSSIEKKIEEITSQIIQISNEITLIRNEIAQIKQ |
| incorporating a | |
| multimerization leucine | |
| zipper domain | |
| (SEQ ID NO: 8) | |
| Human IFNβ-IgG1 FC- | MTNKCLLQIALLLCFSTTALSMSYNLLGFLQRSSNFQCQK |
| CD40L (or IFNb-Ig- | LLWQLNGRLEYCLKDRMNFDIPEEIKQLQQFQKEDAAL |
| CD40L or IFNb-Fc- | TIYEMLQNIFAIFRQDSSSTGWNETIVENLLANVYHQIN |
| CD40L in the Figures) | HLKTVLEEKLEKEDFTRGKLMSSLHLKRYYGRILHYLK |
| (SEQ ID NO: 9) | AKEYSHCAWTIVRVEILRNFYFINRLTGYLRNSGGTSGS |
| | TSGTGSDDKTHTCPPCPAPELLGGPSVFLFPPKPKDT |
| | LMISRTPEVTCVVVDVSHEDPEVKFNWYVDGVEVHN |
| | AKTKPREEQYNSTYRVVSVLTVLHQDWLNGKEYKC |
| | KVSNKALPAPIEKTISKAKGQPREPQVYTLPPSRDEL |
| | TKNQVSLTCLVKGFYPSDIAVEWESNGQPENNYKTT |
| | PPVLDSDGSFFLYSKLTVDKSRWQQGNVFSCSVMHE |
| | ALHNHYTQKSLSLSPGKSGGTSGSTSGTGSTMQKGD |
| | QNPQIAAHVISEASSKTTSVLQWAEKGYYTMSNNLVTL |
| | ENGKQLTVKRQGLYYIYAQVTFCSNREASSQAPFIASLC |
| | LKSPGRFERILLRAANTHSSAKPCGQQS1HLGGVFELQP |
| | GASVFVNVTDPSQVSHGTGFTSFGLLKL |
| Human IFNβ-Linker- | MTNKCLLQIALLLCFSTTALSMSYNLLGFLQRSSNFQCQK |
| CD40L (or IFNb- | LLWQLNGRLEYCLKDRMNFDIPEEIKQLQQFQKEDAAL |
| CD4OL in the Figures) | TIYEMLQNIFAIFRQDSSSTGWNETIVENLLANVYHQIN |
| (SEQ ID NO: 10) | HLKTVLEEKLEKEDFTRGKLMSSLHLKRYYGRILHYLK |
| | AKEYSHCAWTIVRVEILRNFYFINRLTGYLRNSGGTSGS |
| | TSGTGSTMQKGDQNPQIAAHVISEASSKTTSVLQWAEK |
| | GYYTMSNNLVTLENGKQLTVKRQGLYYIYAQVTFCSN |
| | REASSQAPFIASLCLKSPGRFERILLRAANTHSSAKPCGQ |
| | QSIHLGGVFELQPGASVFVNVTDPSQVSHGTGFTSFGLL |
| | KL |
| Human IFNβ-LZ- | MTNKCLLQIALLLCFSTTALSMSYNLLGFLQRSSNFQCQK |
| CD40L (or IFNb-LZ- | LLWQLNGRLEYCLKDRMNFDIPEEIKQLQQFQKEDAAL |
| CD40L in the Figures) | TIYEMLQNIFAIFRQDSSSTGWNETIVENLLANVYHQIN |
| (LZ = leucine zipper) | HLKTVLEEKLEKEDFTRGKLMSSLHLKRYYGRILHYLK |
| (SEQ ID NO: 11) | AKEYSHCAWTIVRVEILRNFYFINRLTGYLRNSGGTSGS |
| | TSGTGSTVSSIEKKIEEITSQIIQISNEITLIRNEIAQIKQ |
| | MQKGDQNPQIAAHVISEASSKTTSVLQWAEKGYYTMS |
| | NNLVTLENGKQLTVKRQGLYYIYAQVTFCSNREASSQA |
| | PFIASLCLKSPGRFERILLRAANTHSSAKPCGQQSIHLGG |
| | VFELQPGASVFVNVTDPSQVSHGTGFTSFGLLKL |
| Human IFNβ-IgG1 FC- | MTNKCLLQIALLLCFSTTALSMSYNLLGFLQRSSNFQCQK |
| TWEAK (or IFNb-Ig- | LLWQLNGRLEYCLKDRMNFDIPEEIKQLQQFQKEDAAL |
| TWEAK or hIFNb- | TIYEMLQNIFAIFRQDSSSTGWNETIVENLLANVYHQIN |
| LhIgG1-LhT weak in | HLKTVLEEKLEKEDFTRGKLMSSLHLKRYYGRILHYLK |
| Figures) | AKEYSHCAWTIVRVEILRNFYFINRLTGYLRNSGGTSGS |
| (SEQ ID NO: 12) | TSGTGSDDKTHTCPPCPAPELLGGPSVFLFPPKPKDT |
| | LMISRTPEVTCVVVDVSHEDPEVKFNWYVDGVEVHN |
| | AKTKPREEQYNSTYRVVSVLTVLHQDWLNGKEYKC |
| | KVSNKALPAPIEKTISKAKGQPREPQVYTLPPSRDEL |
| | TKNQVSLTCLVKGFYPSDIAVEWESNGQPENNYKTT |
| | PPVLDSDGSFFLYSKLTVDKSRWQQGNVFSCSVMHE |
| | ALHNHYTQKSLSLSPGKSGGTSGSTSGTGSTMKGRK |
| | TRARRAIAAHYEVHPRPGQDGAQAGVDGTVSGWEEAR |
| | INSSSPLRYNRQIGEFIVTRAGLYYLYCQVHFDEGKAVY |
| | LKLDLLVDGVLALRCLEEFSATAASSLGPQLRLCQVSGL |
| | LALRPGSSLRIRTLPWAHLKAAPFLTYFGLFQVH |
| Human IFNβ-Linker- | MTNKCLLQIALLLCFSTTALSMSYNLLGFLQRSSNFQCQK |
| TWEAK (or IFNb- | LLWQLNGRLEYCLKDRMNFDIPEEIKQLQQFQKEDAAL |
| TWEAK in the | TIYEMLQNIFAIFRQDSSSTGWNETIVENLLANVYHQIN |
| Figures) | HLKTVLEEKLEKEDFTRGKLMSSLHLKRYYGRILHYLK |
| (SEQ ID NO: 13) | AKEYSHCAWTIVRVEILRNFYFINRLTGYLRNSGGTSGS |
| | TSGTGSTMKGRKTRARRAIAAHYEVHPRPGQDGAQAG |
| | VDGTVSGWEEARINSSSPLRYNRQIGEFIVTRAGLYYLY |
| | CQVHFDEGKAVYLKLDLLVDGVLALRCLEEFSATAASS |
| | LGPQLRLCQVSGLLALRPGSSLRIRTLPWAHLKAAPFLT |
| | YFGLFQVH |
| Human IFNβ-LZ- | MTNKCLLQIALLLCFSTTALSMSYNLLGFLQRSSNFQCQK |
| TWEAK (or IFNb-LZ- | LLWQLNGRLEYCLKDRMNFDIPEEIKQLQQFQKEDAAL |
| TWEAK in the | TIYEMLQNIFAIFRQDSSSTGWNETIVENLLANVYHQIN |
| Figures) | HLKTVLEEKLEKEDFTRGKLMSSLHLKRYYGRILHYLK |
| (SEQ ID NO: 14) | AKEYSHCAWTIVRVEILRNFYFINRLTGYLRNSGGTSGS |
| | TSGTGSTVSSIEKKIEEITSQIIQISNEITLIRNEIAQIKQ |
| | MKGRKTRARRAIAAHYEVHPRPGQDGAQAGVDGTVSG |
| | WEEARINSSSPLRYNRQIGEFIVTRAGLYYLYCQVHFDE |
| | GKAVYLKLDLLVDGVLALRCLEEFSATAASSLGPQLRL |
| | CQVSGLLALRPGSSLRIRTLPWAHLKAAPFLTYFGLFQV |
| | H |
| Human IFNβ-IgG1 FC- | MTNKCLLQIALLLCFSTTALSMSYNLLGFLQRSSNFQCQK |
| LIGHT (or IFNb-Ig- | LLWQLNGRLEYCLKDRMNFDIPEEIKQLQQFQKEDAAL |
| LIGHT or hIFNb- | TIYEMLQNIFAIFRQDSSSTGWNETIVENLLANVYHQIN |
| LhIgG1-LhLight in the | HLKTVLEEKLEKEDFTRGKLMSSLHLKRYYGRILHYLK |
| Figures) | AKEYSHCAWTIVRVEILRNFYFINRLTGYLRNSGGTSGS |
| (SEQ ID NO: 15) | TSGTGSDDKTHTCPPCPAPELLGGPSVFLFPPKPKDT |
| | LMISRTPEVTCVVVDVSHEDPEVKFNWYVDGVEVHN |
| | AKTKPREEQYNSTYRVVSVLTVLHQDWLNGKEYKC |
| | KVSNKALPAPIEKTISKAKGQPREPQVYTLPPSRDEL |
| | TKNQVSLTCLVKGFYPSDIAVEWESNGQPENNYKTT |
| | PPVLDSDGSFFLYSKLTVDKSRWQQGNVFSCSVMHE |
| | ALHNHYTQKSLSLSPGKSGGTSGSTSGTGSTDGPAGS |
| | WEQLIQERRSHEVNPAAHLTGANSSLTGSGGPLLWETQ |
| | LGLAFLRGLSYHDGALVVTKAGYYYIYSKVQLGGVGC |
| | PLGLASTITHGLYKRTPRYPEELELLVSQQSPCGRATSSS |
| | RVWWDSSFLGGVVHLEAGEKVVVRVLDERLVRLRDGT |
| | RSYFGAFMV |
| Human IFNβ-Linker- | MTNKCLLQIALLLCFSTTALSMSYNLLGFLQRSSNFQCQK |
| LIGHT (or IFNb- | LLWQLNGRLEYCLKDRMNFDIPEEIKQLQQFQKEDAAL |
| LIGHT in the Figures) | TIYEMLQNIFAIFRQDSSSTGWNETIVENLLANVYHQIN |
| (SEQ ID NO: 16) | HLKTVLEEKLEKEDFTRGKLMSSLHLKRYYGRILHYLK |
| | AKEYSHCAWTIVRVEILRNFYFINRLTGYLRNSGGTSGS |
| | TSGTGSTDGPAGSWEQLIQERRSHEVNPAAHLTGANSS |
| | LTGSGGPLLWETQLGLAFLRGLSYHDGALVVTKAGYY |
| | YIYSKVQLGGVGCPLGLASTITHGLYKRTPRYPEELELL |
| | VSQQSPCGRATSSSRVWWDSSFLGGVVHLEAGEKVVV |
| | RVLDERLVRLRDGTRSYFGAFMV |
| Human IFNβ-LZ- | MTNKCLLQIALLLCFSTTALSMSYNLLGFLQRSSNFQCQK |
| LIGHT (or IFNb-LZ- | LLWQLNGRLEYCLKDRMNFDIPEEIKQLQQFQKEDAAL |
| LIGHT in the Figures) | TIYEMLQNIFAIFRQDSSSTGWNETIVENLLANVYHQIN |
| (SEQ ID NO: 17) | HLKTVLEEKLEKEDFTRGKLMSSLHLKRYYGRILHYLK |
| | AKEYSHCAWTIVRVEILRNFYFINRLTGYLRNSGGTSGS |
| | TSGTGSTVSSIEKKIEEITSQIIQISNEITLIRNEIAQIKQ |
| | DGPAGSWEQLIQERRSHEVNPAAHLTGANSSLTGSGGP |
| | LLWETQLGLAFLRGLSYHDGALVVTKAGYYYIYSKVQ |
| | LGGVGCPLGLASTITHGLYKRTPRYPEELELLVSQQSPC |
| | GRATSSSRVWWDSSFLGGVVHLEAGEKVVVRVLDERL |
| | VRLRDGTRSYFGAFMV |
| huIgG1-Fc-tweak (Ig- | MTNKCLLQIALLLCFSTTALSSGGTSGSTSGTGSDDKTHT |
| Tweak or LhIgG1- | CPPCPAPELLGGPSVFLFPPKPKDTLMISRTPEVTCV |
| LhTweak in the text and | VVDVSHEDPEVKFNWYVDGVEVHNAKTKPREEQYN |
| figures) | STYRVVSVLTVLHQDWLNGKEYKCKVSNKALPAPIE |
| (SEQ ID NO: 18) | KTISKAKGQPREPQVYTLPPSRDELTKNQVSLTCLVK |
| | GFYPSDIAVEWESNGQPENNYKTTPPVLDSDGSFFLY |
| | SKLTVDKSRWQQGNVFSCSVMHEALHNHYTQKSLS |
| | LSPGKSGGTSGSTSGTGSTMKGRKTRARRAIAAHYEV |
| | HPRPGQDGAQAGVDGTVSGWEEARINSSSPLRYNRQIG |
| | EFIVTRAGLYYLYCQVHFDEGKAVYLKLDLLVDGVLAL |
| | RCLEEFSATAASSLGPQLRLCQVSGLLALRPGSSLRIRTL |
| | PWAHLKAAPFLTYFGLFQVH |
| huIgG1-Fc-Light (Ig- | MTNKCLLQIALLLCFSTTALSSGGTSGSTSGTGSDDKTHT |
| Light or LhIgG1-LhLight | CPPCPAPELLGGPSVFLFPPKPKDTLMISRTPEVTCV |
| in the text and figures) | VVDVSHEDPEVKFNWYVDGVEVHNAKTKPREEQYN |
| (SEQ ID NO: 19) | STYRVVSVLTVLHQDWLNGKEYKCKVSNKALPAPIE |
| | KTISKAKGQPREPQVYTLPPSRDELTKNQVSLTCLVK |
| | GFYPSDIAVEWESNGQPENNYKTTPPVLDSDGSFFLY |
| | SKLTVDKSRWQQGNVFSCSVMHEALHNHYTQKSLS |
| | LSPGKSGGTSGSTSGTGSTDGPAGSWEQLIQERRSHEV |
| | NPAAHGANSSLTGSGGPLLWETQLGLAFLRGLSYHDGA |
| | LVVTKAGYYYIYSKVQLGGVGCPLGLASTITHGLYKRT |
| | PRYPEELELLVSQQSPCGRATSSSRVWWDSSFLGGVVH |
| | LEAGEKVVVRVLDERLVRLRDGTRSYFGAFMV |
| mIFNb Fc-mIgG1 | MNNRWILHAAFLLCFSTTALSINYKQLQLQERTNIRKCQE |
| (SEQ ID NO: 20) | LLEQLNGKINLTYRADFKIPMEMTEKMQKSYTAFAIQE |
| | MLQNVFLVFRNNFSSTGWNETIVVRLLDELHQQTVFLK |
| | TVLEEKQEERLTWEMSSTALHLKSYYWRVQRYLKLMK |
| | YNSYAWMVVRAEIFRNFLIIRRLTRNFQNSGGTSGSTS |
| | GTGSVRSGCKPCICTVPEVSSVFIFPPKPKDVLTITLT |
| | PKVTCVVVDISKDDPEVQFSWFVDDVEVHTAQTQPR |
| | EEQFNSTFRSVSELPIMHQDWLNGKEFKCRVNSAAF |
| | PAPIEKTISKTKGRPKAPQVYTIPPPKEQMAKDKVSL |
| | TCMITDFFPEDITVEWQWNGQPAENYKNTQPIMDTD |
| | GSYFVYSKLNVQKSNWEAGNTFTCSVLHEGLHNHH |
| | TEKSLSHSPG |
| Fc-mIgG1 mCD40L | MNNRWILHAAFLLCFSTTALSSGGTSGSTSGTGSVRSGC |
| (SEQ ID NO: 21) | KPCICTVPEVSSVFIFPPKPKDVLTITLTPKVTCVVVDI |
| | SKDDPEVQFSWFVDDVEVHTAQTQPREEQFNSTFRS |
| | VSELPIMHQDWLNGKEFKCRVNSAAFPAPIEKTISKT |
| | KGRPKAPQVYTIPPPKEQMAKDKVSLTCMITDFFPE |
| | DITVEWQWNGQPAENYKNTQPIMDTDGSYFVYSKL |
| | NVQKSNWEAGNTFTCSVLHEGLHNHHTEKSLSHSPG |
| | KSGGTSGSTSGTGSTMQRGDEDPQIAAHVVSEANSNA |
| | ASVLQWAKKGYYTMKSNLVMLENGKQLTVKREGLYY |
| | VYTQVTFCSNREPSSQRPFIVGLWLKPSSGSERILLKAAN |
| | THSSSQLCEQQSVHLGGVFELQAGASVFV |
| | NVTEASQVIHRVGFSSFGLLKL |
| Fc-mIgG1 | MNNRWILHAAFLLCFSTTALSSGGTSGSTSGTGSVRSGC |
| (SEQ ID NO: 22) | KPCICTVPEVSSVFIFPPKPKDVLTITLTPKVTCVVVDI |
| | SKDDPEVQFSWFVDDVEVHTAQTQPREEQFNSTFRS |
| | VSELPIMHQDWLNGKEFKCRVNSAAFPAPIEKTISKT |
| | KGRPKAPQVYTIPPPKEQMAKDKVSLTCMITDFFPE |
| | DITVEWQWNGQPAENYKNTQPIMDTDGSYFVYSKL |
| | NVQKSNWEAGNTFTCSVLHEGLHNHHTEKSLSHSPG |
| | K |
| mCD40L soluble form | MQRGDEDPQIAAHVVSEANSNAASVLQWAKKGYYTM |
| (SEQ ID NO: 23) | KSNLVMLENGKQLTVKREGLYYVYTQVTFCSNREPSSQ |
| | RPFIVGLWLKPSSGSERILLKAANTHSSSQLCEQQSVHL |
| | GGVFELQAGASVFVNVTEASQVIHRVGFSSFGLLKL |
| mIFNb | MNNRWILHAAFLLCFSTTALSINYKQLQLQERTNIRKCQE |
| (SEQ ID NO: 24) | LLEQLNGKINLTYRADFKIPMEMTEKMQKSYTAFAIQE |
| | MLQNVFLVFRNNFSSTGWNETIVVRLLDELHQQTVFLK |
| | TVLEEKQEERLTWEMSSTALHLKSYYWRVQRYLKLMK |
| | YNSYAWMVVRAEIFRNFLIIRRLTRNFQN |
| IFNa-G4S-huIgG1-LN2- | MALTFALLVALLVLSCKSSCSVGCDLPQTHSLGSRRTLML |
| huCD40L | LAQMRKISLFSCLKDRHDFGFPQEEFGNQFQKAETIPVL |
| (IFNa-huIgG1-hu-CD40 | HEMIQQIFNLFSTKDSSAAWDETLLDKFYTELYQQLND |
| in the text and figures) | LEACVIQGVGVTETPLMKEDSILAVRKYFQRITLYLKEK |
| (SEQ ID NO: 25) | KYSPCAWEVVRAEIMRSFSLSTNLQESLRSKEGGGGSG |
| | GGGSGGGGSDKTHTCPPCPAPELLGGPSVFLFPPKP |
| | KDTLMISRTPEVTCVVVDVSHEDPEVKFNWYVDGVE |
| | VHNAKTKPREEQYNSTYRVVSVLTVLHQDWLNGKE |
| | YKCKVSNKALPAPIEKTISKAKGQPREPQVYTLPPSR |
| | DELTKNQVSLTCLVKGFYPSDIAVEWESNGQPENNY |
| | KTTPPVLDSDGSFFLYSKLTVDKSRWQQGNVFSCSV |
| | MHEALHNHYTQKSLSLSPGKGSPAPDPAPDPSGMQK |
| | GDQNPQIAAHVISEASSKTTSVLQWAEKGYYTMSNNLV |
| | TLENGKQLTVKRQGLYYIYAQVTFCSNREASSQAPFIAS |
| | LCLKSPGRFERILLRAANTHSSAKPCGQQSIHLGGVFEL |
| | QPGASVFVNVTDPSQVSHGTGFTSFGLLKL |
| Lz-Light | [symbol] SSIEKKIEEITSQIIQISNEITLIR |
| (SEQ ID NO: 26) | NEIAQIKQDGPAGSWEQLIQERRSHEVNPAAHLTGA |
| | NSSLTGSGGPLLWETQLGLAFLRGLSYHDGALVVTK |
| | AGYYYIYSKVQLGGVGCPLGLASTITHGLYKRTPRY |
| | PEELELLVSQQSPCGRATSSSRVWWDSSFLGGVVHL |
| | EAGEKVVVRVLDER VRLRDGTRSYFGAFMV |
| Signal peptide 1 | MTNKCLLQIALLLCFSTTALS |
| (SEQ ID NO: 29) | |
| Signal peptide 2 | MNNRWILHAAFLLCFSTTALS |
| (SEQ ID NO: 30) | |
| Signal peptide 3 | MALTFALLVALLVLSCKS |
| (SEQ ID NO: 31) | |
| Signal peptide 4 | MSGGTSGSTSGTGST |
| (SEQ ID NO: 32) | |
| IFNa2 a | MALTFALLVALLVLSCKSSCSVGCDLPQTHSLGSRRTLML |
| (SEQ ID NO: 33) | LAQMRKISLFSCLKDRHDFGFPQEEFGNQFQKAETIPVL |
| | HEMIQQIFNLFSTKDSSAAWDETLLDKFYTELYQQLND |
| | LEACVIQGVGVTETPLMKEDSILAVRKYFQRITLYLKEK |
| | KYSPCAWEVVRAEIMRSFSLSTNLQESLRSKE |

The components as presented in table 3 above may be used to prepare a medicament or pharmaceutical composition as disclosed herein.

In preferred embodiments, the active agents consist of polypeptides derived from those specified in Table 3 above, and especially from the polypeptides of SEQ IDs NO. 1, 2, 3, 4, 5, 9, 10, 11, 12, 13, 14, 15, 16, 17, 18, 19, 20, 21, 22, 23, 24, 25, 26 and 33 above, and are devoid of any signal peptide when they are used for treating an HBV infection. Indeed, the signal peptides that are initially present in the sequences of the polypeptides when these are synthesized are then cleaved.

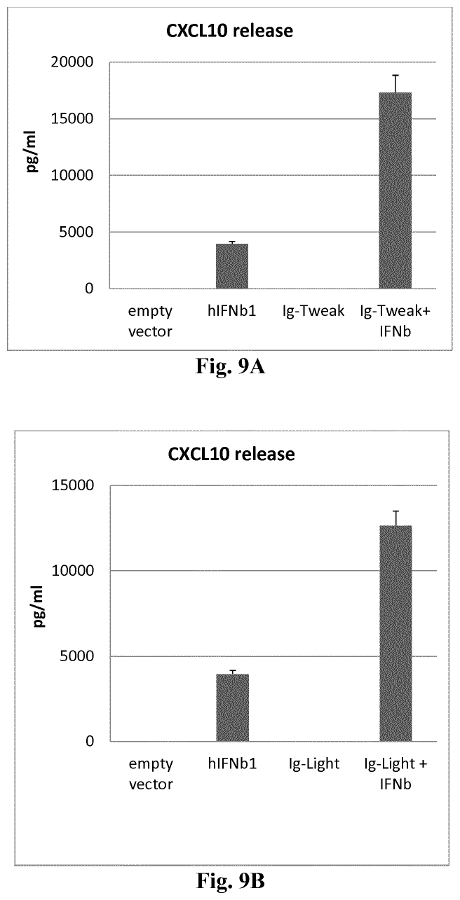

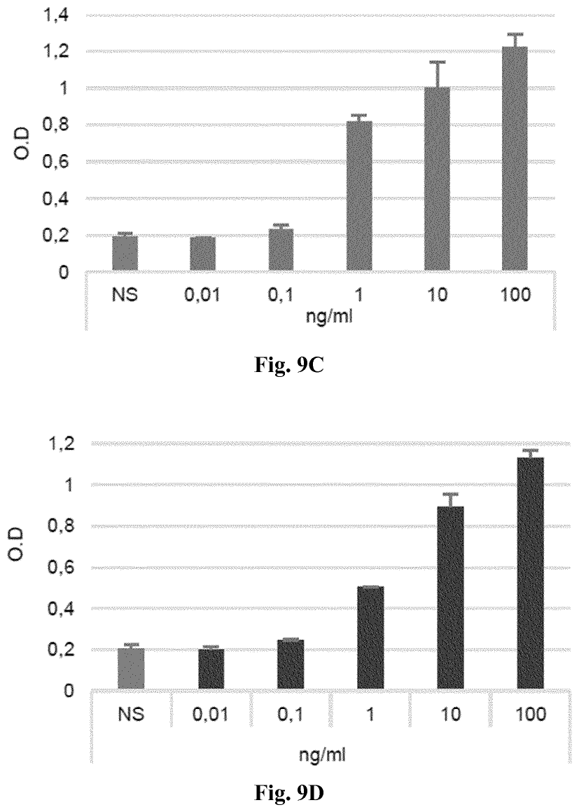

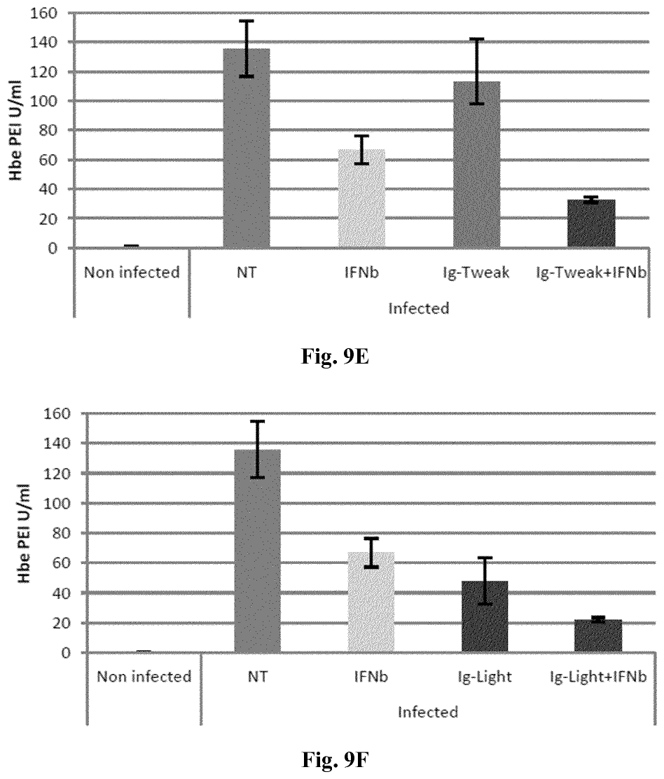

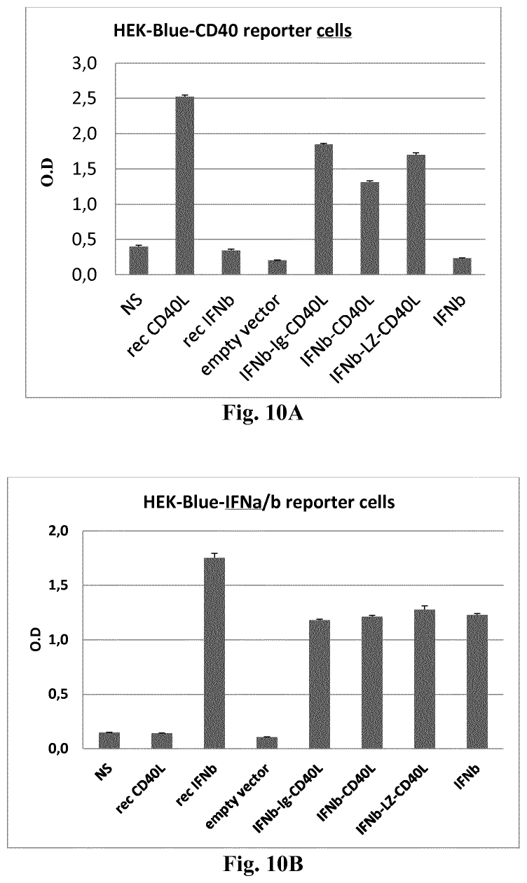

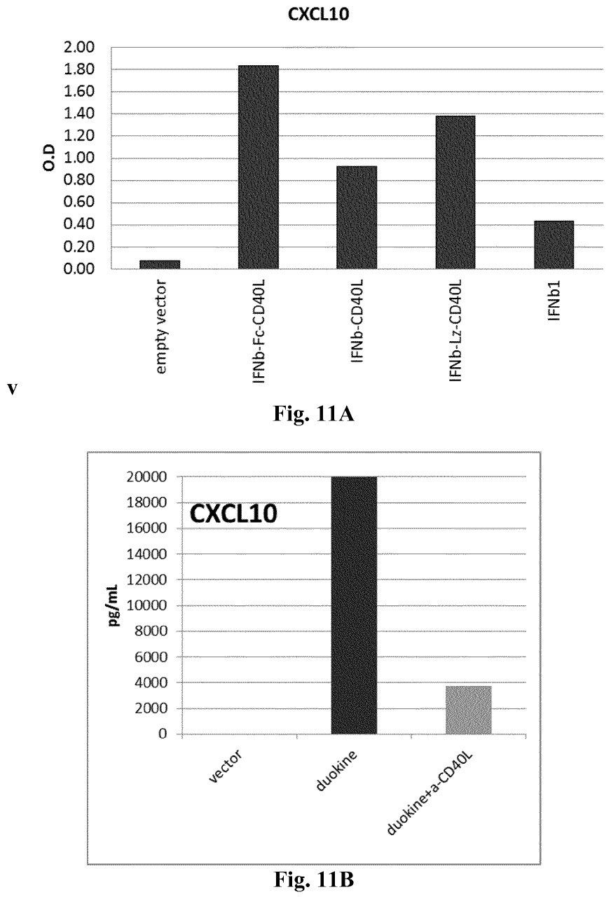

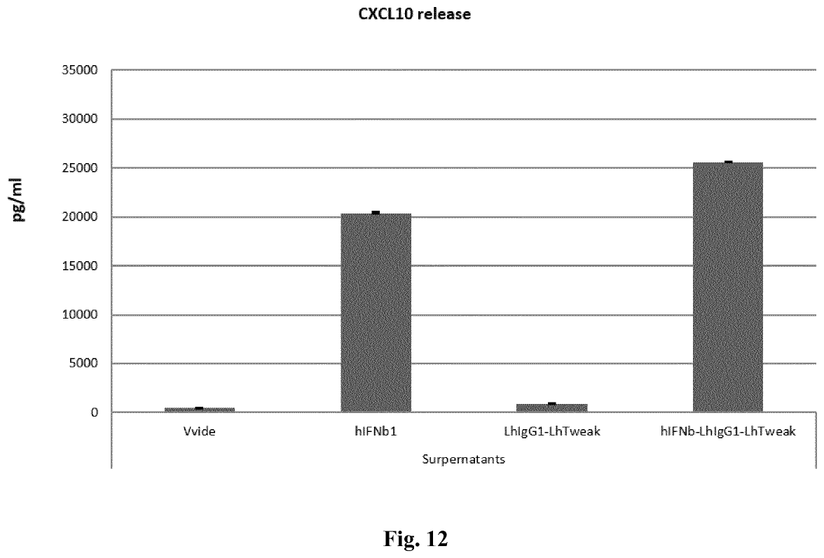

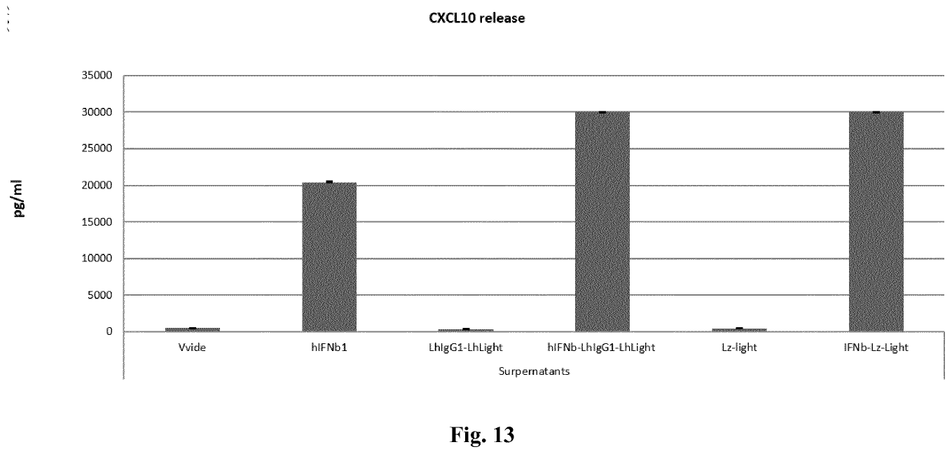

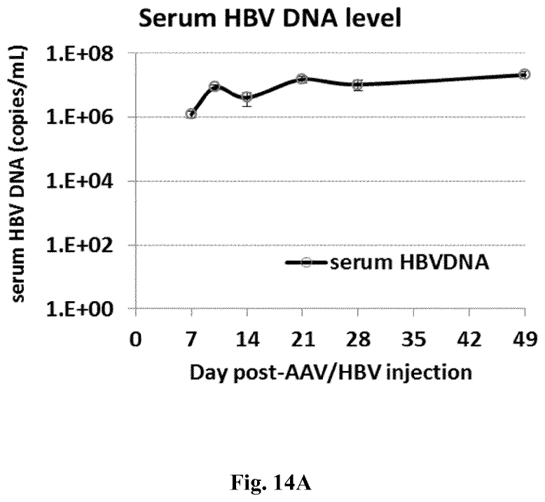

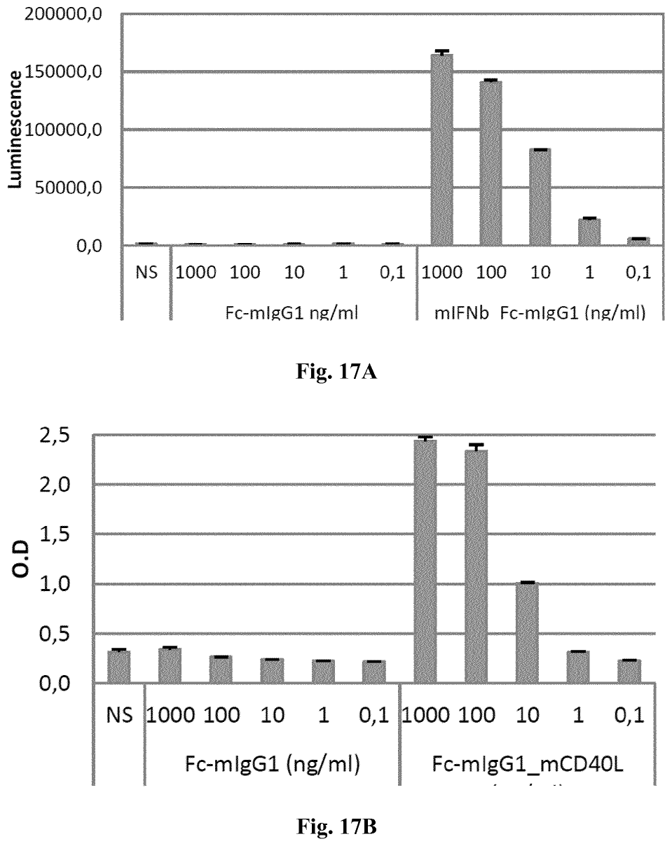

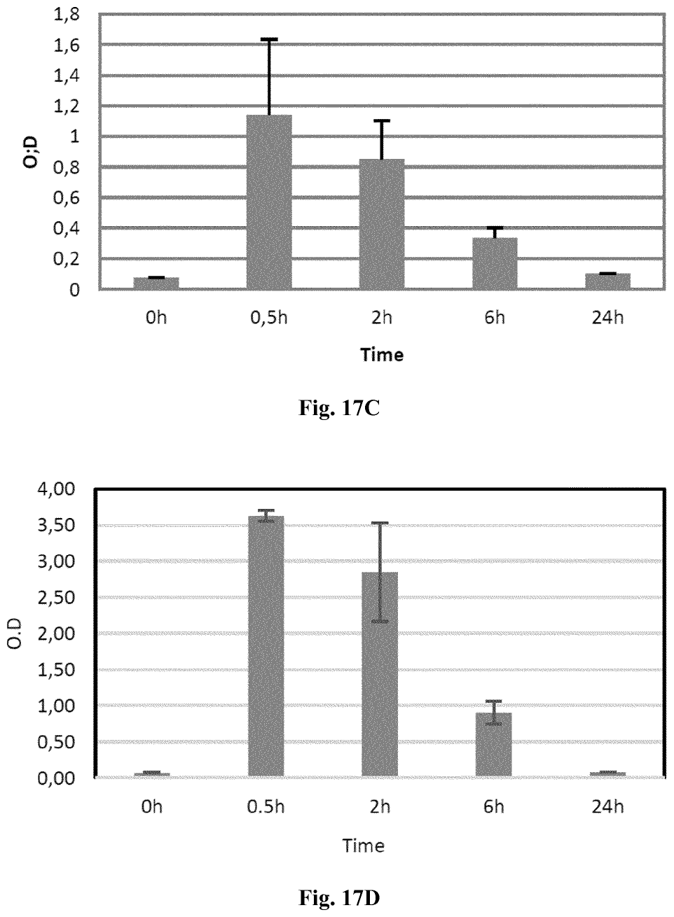

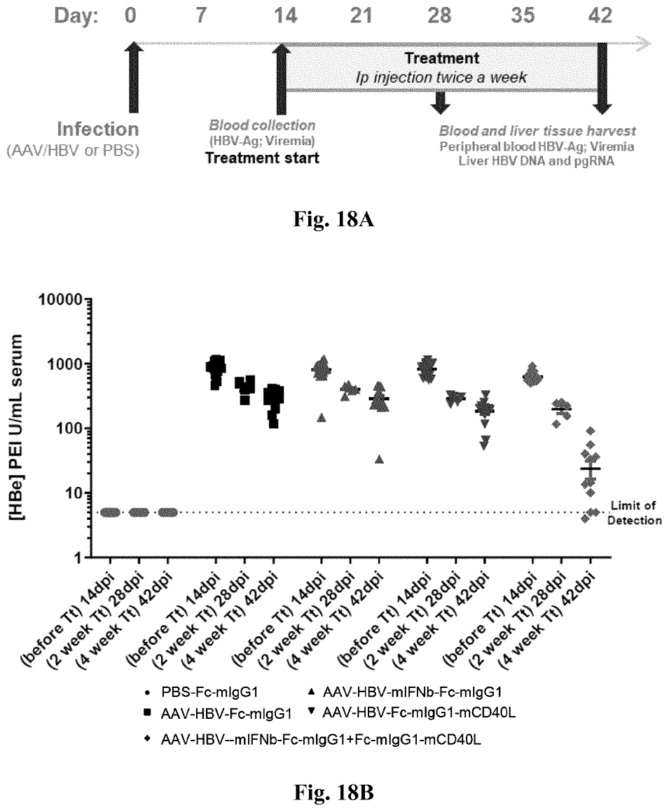

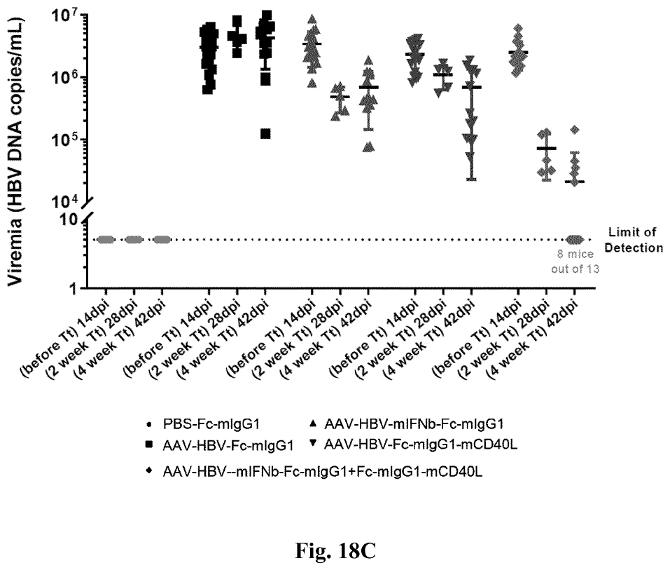

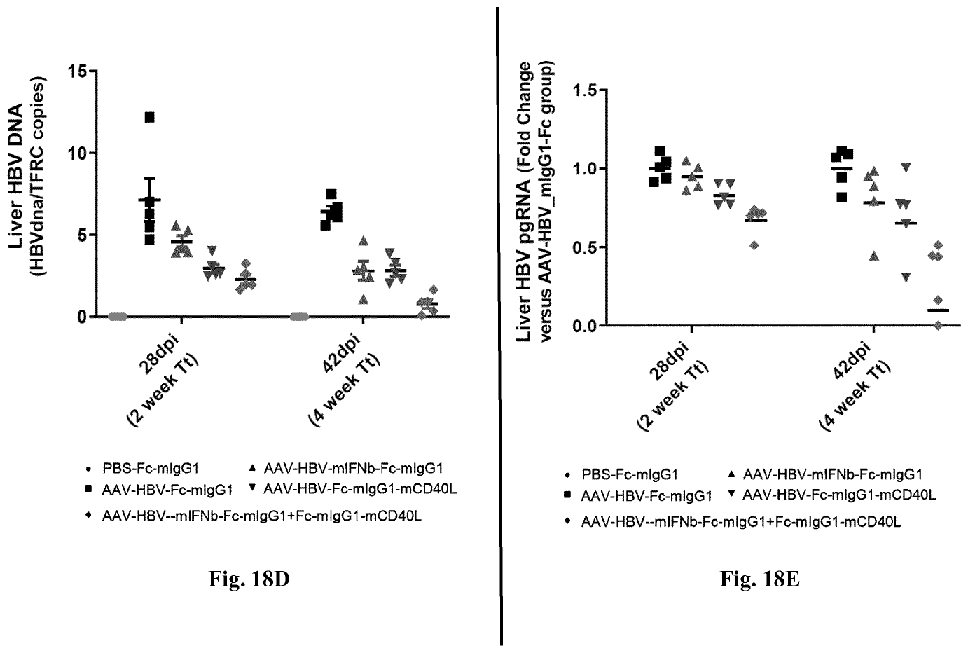

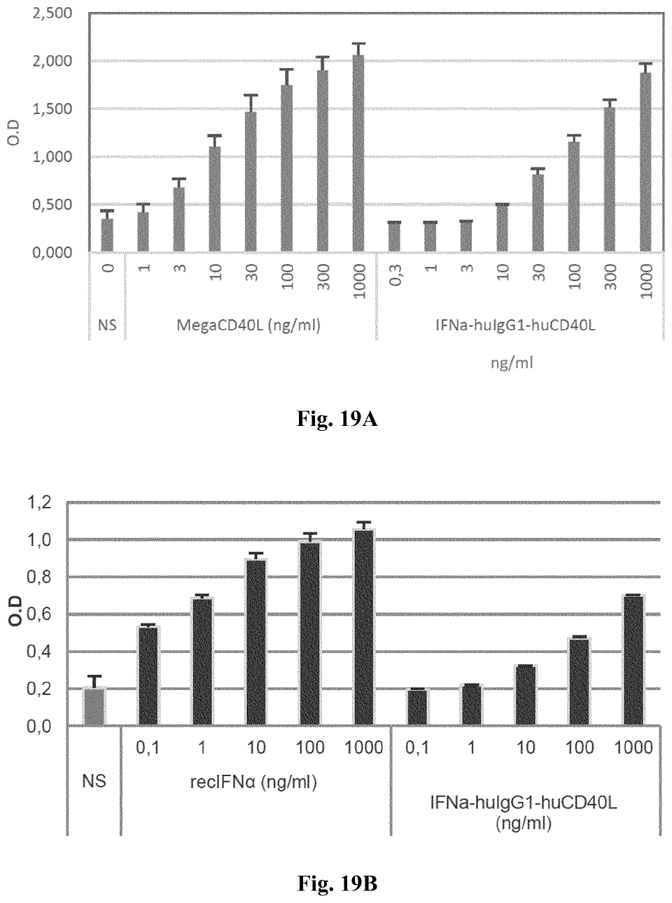

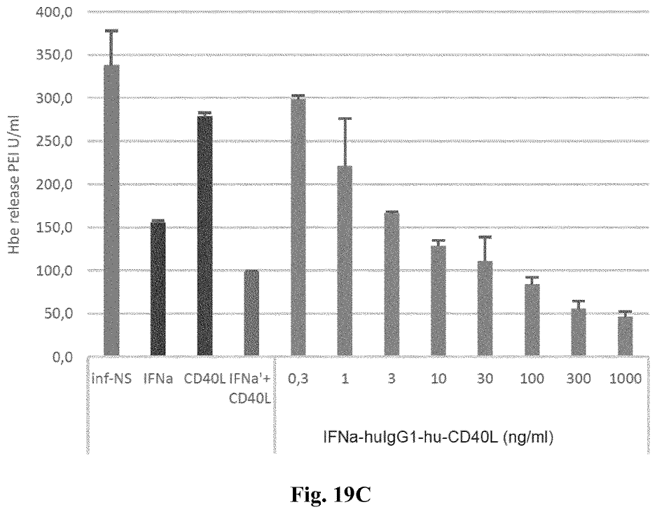

TABLE 4
Correspondence between construct names used in the figures
(or in the text) and SEQ ID NOs

| Fig. NO | Construct name in the figures | SEQ ID NO |
|---|---|---|
| FIG. 10A/B | IFNb-Ig-CD40L | SEQ ID NO: 9 |
| | IFNb-CD40L | SEQ ID NO: 10 |
| | IFNb-LZ-CD40L | SEQ ID NO: 11 |
| FIG. 11A | IFNb-Fc-CD40L (IFNb-Ig-CD40L) | SEQ ID NO: 9 |
| | IFNb-CD40L | SEQ ID NO: 10 |
| | IFNb-LZ-CD40L | SEQ ID NO: 11 |
| FIG. 11B | duokine | SEQ ID NO: 9 |
| FIG. 9A | hIFNb1 | SEQ ID NO: 1 |
| | Ig-Tweak | SEQ ID NO: 18 |
| FIG. 9B | hIFNb1 | SEQ ID NO: 1 |
| | Ig-LIGHT | SEQ ID NO: 19 |
| FIG. 12 | hIFNb1 | SEQ ID NO: 1 |
| | LhIgG1-LhTweak (Ig-Tweak) | SEQ ID NO: 18 |
| | hIFNb-LhIgG1-LhTweak (IFNb-Ig-Tweak) | SEQ ID NO: 12 |
| FIG. 13 | hIFNb1 | SEQ ID NO: 1 |
| | LhIgG1-LhLight (Ig-Light) | SEQ ID NO: 19 |
| | hIFNb-LhIgG1-LhLight (IFNb-Ig-Light) | SEQ ID NO: 15 |
| | Lz-Light | SEQ ID NO: 26 |
| | IFNb-LZ-Light | seq ID NO: 17 |
| FIG. 17 and | mIFNb Fc-mIgG1 | SEQ ID NO: 20 |
| FIG. 18 | Fc-mIgG1_mCD40L | SEQ ID NO: 21 |
| | Fc-mIgG1 | SEQ ID NO: 22 |
| FIG. 19 | IFNa-huIgG1-hu-CD40L | SEQ ID NO: 25 |

Nucleic Acids and Expression Vectors

In one aspect, a combination of polynucleotides encoding a TNFRSF agonist or a functional fragment thereof and an IFN agent or functional fragment thereof is provided. Methods of making a combination of TNFRSF agonist or a functional fragment thereof and an IFN agent or a functional fragment thereof comprising expressing these polynucleotides are also provided.

Polynucleotides encoding a TNFRSF agonist or a functional fragment thereof, an IFN agent or a functional fragment of an IFN agent as disclosed herein are typically inserted in an expression vector for introduction into host cells that may be used to produce the desired quantity of the claimed antibodies, or immunoadhesins. Accordingly, in certain aspects, the invention provides expression vectors comprising polynucleotides disclosed herein and host cells comprising these vectors and polynucleotides.

The term “vector” or “expression vector” is used herein for the purposes of the specification and claims, to mean vectors used in accordance with the present invention as a vehicle for introducing into and expressing a desired gene in a cell. As known to those skilled in the art, such vectors may easily be selected from the group consisting of plasmids, phages, viruses and retroviruses. In general, vectors compatible with the instant invention will comprise a selection marker, appropriate restriction sites to facilitate cloning of the desired gene and the ability to enter and/or replicate in eukaryotic or prokaryotic cells.

Numerous expression vector systems may be employed for the purposes of this invention. For example, one class of vector utilizes DNA elements which are derived from animal viruses such as bovine papilloma virus, polyoma virus, adenovirus, vaccinia virus, baculovirus, retroviruses (RSV, MMTV or MOMLV), or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by co-transformation. Additional elements may also be needed for optimal synthesis of mRNA. These elements may include signal sequences, splice signals, as well as transcriptional promoters, enhancers, and termination signals. In some embodiments the cloned variable region genes are inserted into an expression vector along with the heavy and light chain constant region genes (such as human genes) synthesized as discussed above.

In other embodiments, a TNFRSF agonist or a functional fragment thereof, an IFN agent or a functional fragment of an IFN agent as described herein may be expressed using polycistronic constructs. In such expression systems, multiple gene products of interest such as TNFRSF agonists or a functional fragment thereof, IFNs or functional fragments of IFNs may be produced from a single polycistronic construct. These systems advantageously use an internal ribosome entry site (IRES) to provide relatively high levels of polypeptides in eukaryotic host cells. Compatible IRES sequences are disclosed in U.S. Pat. No. 6,193,980, which is incorporated by reference herein. Those skilled in the art will appreciate that such expression systems may be used to effectively produce the full range of polypeptides disclosed in the instant application.

More generally, once a vector or DNA sequence encoding a TNFRSF agonist or a functional fragment thereof, an IFN agent or a functional fragment of an IFN agent of the present disclosure has been prepared, the expression vector may be introduced into an appropriate host cell. That is, the host cell may be transformed. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, e.g., Ridgway, A. A. G. “Mammalian Expression Vectors” Chapter 24.2, pp. 470-472 Vectors, Rodriguez and Denhardt, Eds. (Butterworths, Boston, Mass. 1988). The transformed cells are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or fluorescence-activated cell sorter analysis (FACS), immunohistochemistry and the like.

As used herein, the term “transformation” shall be used in a broad sense to refer to the introduction of DNA into a recipient host cell that changes the genotype and consequently results in a change in the recipient cell.

Along those same lines, “host cells” refer to cells that have been transformed with vectors constructed using recombinant DNA techniques and encoding at least one heterologous gene. In descriptions of processes for isolation of polypeptides from recombinant hosts, the terms “cell” and “cell culture” are used interchangeably to denote the source of antibody unless it is clearly specified otherwise. In other words, recovery of polypeptide from the “cells” may mean either from spun down whole cells, or from the cell culture containing both the medium and the suspended cells.

In one embodiment, the host cell line used for expression of a TNFRSF agonist or a functional fragment thereof, an IFN agent or a functional fragment of an IFN agent is of eukaryotic or prokaryotic origin. In one embodiment, the host cell line used for expression of a TNFRSF agonist or a functional fragment thereof, an IFN agent or a functional fragment of an IFN agent is of bacterial origin. In one embodiment, the host cell line used for expression of a TNFRSF agonist or a functional fragment thereof, an IFN agent or a functional fragment of an IFN agent is of mammalian origin; those skilled in the art can determine particular host cell lines which are best suited for the desired gene product to be expressed therein. Exemplary host cell lines include, but are not limited to, DG44 and DUXB11 (Chinese Hamster Ovary lines, DHFR minus), HELA (human cervical carcinoma), CVI (monkey kidney line), COS (a derivative of CVI with SV40 T antigen), R1610 (Chinese hamster fibroblast) BALBC/3T3 (mouse fibroblast), HAK (hamster kidney line), SP2/O (mouse myeloma), BFA-1c1BPT (bovine endothelial cells), RAJI (human lymphocyte), 293 (human kidney). In one embodiment, the cell line provides for altered glycosylation, e.g., afucosylation, of the antibody expressed therefrom (e.g., PER.C6® (Crucell) or FUT8-knock-out CHO cell lines (POTELLIGENT™ cells) (Biowa, Princeton, N.J.)). In one embodiment NS0 cells may be used. Host cell lines are typically available from commercial services, the American Tissue Culture Collection or from published literature.

In vitro production allows scale-up to give large amounts of the desired TNFRSF agonists or a functional fragment thereof, IFNs or functional fragments of IFNs. Techniques for mammalian cell cultivation under tissue culture conditions are known in the art and include homogeneous suspension culture, e.g., in an airlift reactor or in a continuous stirrer reactor, or immobilized or entrapped cell culture, e.g., in hollow fibers, microcapsules, on agarose microbeads or ceramic cartridges. If necessary and/or desired, a solution of a TNFRSF agonist or a functional fragment thereof, an IFN agent or a functional fragment of an IFN agent, can be purified by the customary chromatography methods, for example gel filtration, ion-exchange chromatography, chromatography over DEAE-cellulose and/or (immuno-) affinity chromatography.

One or more genes encoding TNFRSF agonists or a functional fragment thereof, IFNs or functional fragments of IFNs can also be expressed in non-mammalian cells such as bacteria or yeast or plant cells. In this regard it will be appreciated that various unicellular non-mammalian microorganisms such as bacteria can also be transformed; i.e. those capable of being grown in cultures or fermentation. Bacteria, which are susceptible to transformation, include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, TNFRSF agonists or a functional fragment of thereof, IFNs or functional fragments of IFNs can become part of inclusion bodies. The TNFRSF agonists or a functional fragment thereof, IFNs or a functional fragment of IFNs must be isolated and purified.

In addition to prokaryotes, eukaryotic microbes may also be used. Saccharomyces cerevisiae, or common baker's yeast, is the most commonly used among eukaryotic microorganisms although a number of other strains are commonly available. For expression in Saccharomyces, the plasmid YRp7, for example, (Stinchcomb et al., Nature, 282:39 (1979); Kingsman et al., Gene, 7:141 (1979); Tschemper et al., Gene, 10:157 (1980)) is commonly used. This plasmid already contains the TRP1 gene which provides a selection marker for a mutant strain of yeast lacking the ability to grow in tryptophan, for example ATCC No. 44076 or PEP4-1 (Jones, Genetics, 85:12 (1977)). The presence of the trp1 lesion as a characteristic of the yeast host cell genome then provides an effective environment for detecting transformation by growth in the absence of tryptophan.

Therapeutic Vectors

A nucleic acid sequence encoding a TNFRSF agonist or a functional fragment thereof, an IFN agent or a functional fragment of an IFN agent, can be inserted into a vector and used as a therapeutic vector, e.g., a vector that expresses a TNFRSF agonist or a functional fragment thereof, an IFN agent or a functional fragment of an IFN agent. The construction of suitable, functional expression constructs and therapeutic expression vectors is known to one of ordinary skill in the art.

Therapeutic vectors can be delivered to a subject by, for example, intravenous injection, local administration (see U.S. Pat. No. 5,328,470) or by stereotactic injection (see, e.g., Chen et al., PNAS 91:3054-3057 (1994)). The pharmaceutical preparation of a therapeutic vector can include the vector in an acceptable diluent.

A TNFRSF agonist—or a functional fragment—of thereof, an IFN agent—or a functional fragment of an IFN agent-encoding nucleic acid, can be incorporated into a gene construct to be used as a part of a therapy protocol to deliver nucleic acids encoding a TNFRSF agonist or a functional fragment thereof, an IFN agent or a functional fragment of an IFN agent. Expression vectors for in vivo transfection and expression of a TNFRSF agonist or a functional fragment thereof, an IFN agent or a functional fragment of an IFN agent are provided.

Expression constructs of such components may be administered in any biologically effective carrier, e.g., any formulation or composition capable of effectively delivering the component nucleic acid sequence to cells in vivo, as are known to one of ordinary skill in the art. Approaches include, but are not limited to, insertion of the subject nucleic acid sequence in viral vectors including, but not limited to, recombinant retroviruses, adenovirus, adeno-associated virus and herpes simplex virus-1, recombinant bacterial or eukaryotic plasmids and the like.

Retrovirus vectors and adeno-associated viral vectors can be used as a recombinant delivery system for the transfer of exogenous nucleic acid sequences in vivo, particularly into humans. Such vectors provide efficient delivery of genes into cells, and the transferred nucleic acids can be stably integrated into the chromosomal DNA of the host.

The development of specialized cell lines (termed “packaging cells”) which produce only replication-defective retroviruses has increased the utility of retroviruses for gene therapy, and defective retroviruses are characterized for use in gene transfer for gene therapy purposes (for a review see, e.g., Miller, Blood 76:271-78 (1990)). A replication-defective retrovirus can be packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, et al., (eds.) Greene Publishing Associates, (1989), Sections 9.10-9.14, and other standard laboratory manuals. Non-limiting examples of suitable retroviruses include pLJ, pZIP, pWE and pEM which are known to those of ordinary skill in the art. Examples of suitable packaging virus lines include *Crip, *Cre, *2 and *Am. (See, for example, Eglitis, et al., Science 230:1395-1398 (1985); Danos and Mulligan, Proc. Natl. Acad. Sci. USA 85:6460-6464 (1988); Wilson, et al., Proc. Natl. Acad. Sci. USA 85:3014-3018 (1988); Armentano, et al., Proc. Natl. Acad. Sci. USA 87:6141-6145 (1990); Huber, et al., Proc. Natl. Acad. Sci. USA 88:8039-8043 (1991); Ferry, et al., Proc. Natl. Acad. Sci. USA 88:8377-8381 (1991); Chowdhury, et al., Science 254:1802-1805 (1991); van Beusechem, et al., Proc. Natl. Acad. Sci. USA 89:7640-7644 (1992); Kay, et al., Human Gene Therapy 3:641-647 (1992); Dai, et al., Proc. Natl. Acad. Sci. USA 89:10892-10895 (1992); Hwu, et al., J. Immunol. 150:4104-4115 (1993); U.S. Pat. No. 4,868,116; U.S. Pat. No. 4,980,286; PCT Application WO 89/07136; PCT Application WO 89/02468; PCT Application WO 89/05345; and PCT Application WO 92/07573).

In another embodiment, adenovirus-derived delivery vectors are provided. The genome of an adenovirus can be manipulated such that it encodes and expresses a gene product of interest but is inactivated in terms of its ability to replicate in a normal lytic viral life cycle. See, for example, Berkner, et al., BioTechniques 6:616 (1988); Rosenfeld, et al., Science 252:431-434 (1991); and Rosenfeld, et al., Cell 68:143-155 (1992). Suitable adenoviral vectors derived from the adenovirus strain Ad type 5 d1324 or other strains of adenovirus (e.g., Ad2, Ad3, Ad7 etc.) are known to those of ordinary skill in the art. Recombinant adenoviruses can be advantageous in certain circumstances in that they are not capable of infecting non-dividing cells and can be used to infect a wide variety of cell types, including epithelial cells (Rosenfeld, et al. (1992), supra). Furthermore, the virus particle is relatively stable and amenable to purification and concentration and, as above, can be modified so as to affect the spectrum of infectivity. Additionally, introduced adenoviral DNA (and foreign DNA contained therein) is not integrated into the genome of a host cell, but remains episomal, thereby avoiding potential problems that can occur as a result of insertional mutagenesis in situ where introduced DNA becomes integrated into the host genome (e.g., retroviral DNA). Moreover, the carrying capacity of the adenoviral genome for foreign DNA is large (up to 8 kilobases) relative to other delivery vectors (Berkner, et al. (1998), supra; Haj-Ahmand and Graham, J. Virol. 57:267 (1986)).

Yet another viral vector system useful for delivery of a nucleic acid sequence encoding a TNFRSF agonist or a functional fragment thereof, an IFN agent or a functional fragment of an IFN agent, is the adeno-associated virus (AAV). AAV is a naturally occurring defective virus that requires another virus, such as an adenovirus or a herpes virus, as a helper virus for efficient replication and a productive life cycle. (For a review see Muzyczka, et al., Curr. Topics in Micro. and Immunol. 158:97-129 (1992)). It is also one of the few viruses that may integrate its DNA into non-dividing cells, and exhibits a high frequency of stable integration (see for example Flotte, et al., Am. J. Respir. Cell. Mol. Biol. 7:349-356 (1992); Samulski, et al., J. Virol. 63:3822-3828 (1989); and McLaughlin, et al., J. Virol. 62:1963-1973 (1989)). Vectors containing as little as 300 base pairs of AAV can be packaged and can integrate. Space for exogenous DNA is limited to about 4.5 kb. An AAV vector such as that described in Tratschin, et al., Mol. Cell. Biol. 5:3251-3260 (1985) can be used to introduce DNA into cells. A variety of nucleic acids have been introduced into different cell types using AAV vectors (see for example Hermonat, et al., Proc. Natl. Acad. Sci. USA 81:6466-6470 (1984); Tratschin, et al., Mol. Cell. Biol. 4:2072-2081 (1985); Wondisford, et al., Mol. Endocrinol. 2:32-39 (1988); Tratschin, et al., J. Virol. 51:611-619 (1984); and Flotte, et al., J. Biol. Chem. 268:3781-3790 (1993)).

In addition to viral transfer methods, non-viral methods can also be employed to cause expression of a nucleic acid sequence encoding a TNFRSF agonist or a functional fragment thereof, an IFN agent or a functional fragment of an IFN agent in the tissue of a subject. Most non-viral methods of gene transfer rely on normal mechanisms used by mammalian cells for the uptake and intracellular transport of macromolecules. In some embodiments, non-viral delivery systems rely on endocytic pathways for the uptake of the subject gene by the targeted cell. Exemplary delivery systems of this type include liposomal derived systems, poly-lysine conjugates, and artificial viral envelopes. Other embodiments include plasmid injection systems such as are described in Meuli, et al., J. Invest. Dermatol. 116 (1):131-135 (2001); Cohen, et al., Gene Ther 7 (22):1896-905 (2000); or Tam, et al., Gene Ther. 7 (21):1867-74 (2000).

In clinical settings, the delivery systems can be introduced into a subject by any of a number of methods, each of which is familiar in the art. For instance, a pharmaceutical preparation of the delivery system can be introduced systemically, e.g., by intravenous injection. Specific transduction of the protein in the target cells occurs predominantly from specificity of transfection provided by the delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the delivery vehicle can be introduced by catheter (see, U.S. Pat. No. 5,328,470) or by stereotactic injection (e.g., Chen, et al., PNAS 91: 3054-3057 (1994)).

The pharmaceutical preparation of the therapeutic construct can consist essentially of the delivery system in an acceptable diluent, or can comprise a slow release matrix in which the delivery vehicle is imbedded. Alternatively, where the complete delivery system can be produced intact from recombinant cells, e.g., retroviral vectors, the pharmaceutical preparation can comprise one or more cells which produce the delivery system.

Methods of Treatment

In one aspect, the invention provides methods of treating a patient in need thereof (e.g., a patient infected with HBV) comprising administering an effective amount of a TNFRSF agonist or a functional fragment thereof or a nucleic acid sequence (e.g., mRNA) that encodes a TNFRSF agonist or a functional fragment thereof, and an effective amount of an IFN agent or a functional fragment of an IFN agent, or a nucleic acid sequence (e.g., mRNA) that encodes an IFN agent or a functional fragment of an IFN agent, as disclosed herein. In certain embodiments, the present disclosure provides kits and methods for the treatment of disorders and/or symptoms, e.g., HBV-related disorders and/or HBV-related symptoms, in a mammalian subject in need of such treatment. In certain exemplary embodiments, the subject is a human.

The TNFRSF agonists or a functional fragment thereof, IFNs or functional fragments of IFNs, or nucleic acid sequences that encode them, of the current disclosure are useful in a number of different applications. For example, in one embodiment, the subject TNFRSF agonists or a functional fragment thereof, IFNs or functional fragments of IFNs, or nucleic acid sequences that encode them, are useful for reducing HBeAg release from an HBV-infected cell. In another embodiment, the subject TNFRSF agonists or a functional fragment thereof, IFNs or functional fragments of IFNs, or nucleic acid sequences that encode them, are useful for reducing pgRNA transcription of cccDNA in an HBV-infected cell.

In another embodiment, the subject TNFRSF agonists or a functional fragment thereof, IFNs or functional fragments of IFNs, or nucleic acid sequences that encode them, are useful for reducing one or more symptoms and/or complications associated with HBV infection, as described herein (infra).

In certain embodiments, the subject TNFRSF agonists or a functional fragment thereof, IFNs or functional fragments of IFNs, or nucleic acid sequences that encode them, are useful for reducing one or more disorders, symptoms and/or complications associated with chronic HBV infection, e.g., chronic inflammation of the liver (chronic hepatitis), leading to cirrhosis over a period of several years; hepatocellular carcinoma (HCC); development of membranous glomerulonephritis (MGN); risk of death; acute necrotizing vasculitis (polyarteritis nodosa), membranous glomerulonephritis, and papular acrodermatitis of childhood (Gianotti-Crosti syndrome); HBV-associated nephropathy (e.g., membranous glomerulonephritis); immune-mediated hematological disorders (e.g., essential mixed cryoglobulinemia, aplastic anemia); and the like.

In certain embodiments, the subject TNFRSF agonists or a functional fragment thereof, IFNs or functional fragments of IFNs, or nucleic acid sequences that encode them, are useful for reducing one or more symptoms and/or complications associated with acute HBV infection, e.g., acute viral hepatitis (which begins with general ill-health, loss of appetite, nausea, vomiting, body aches, mild fever, and dark urine, and then progresses to development of jaundice, fulminant hepatic failure, and/or serum-sickness-like syndrome); loss of appetite; joint and muscle pain; low-grade fever; stomach pain; nausea; vomiting; jaundice; bloated stomach; and the like.

Accordingly, this disclosure also relates to a method of treating one or more disorders, symptoms and/or complications associated with HBV infection in a human or other animal by administering to such human or animal an effective, non-toxic amount of a TNFRSF agonist or a functional fragment thereof and an IFN agent or a functional fragment of an IFN agent, or nucleic acid sequences that encode them. One skilled in the art would be able, by routine experimentation, to determine what an effective, non-toxic amount of a TNFRSF agonist or a functional fragment thereof and an IFN agent or a functional fragment of an IFN agent, or nucleic acid sequences that encode them, would be for the purpose of treating HBV infection.

For example, a therapeutically active amount of a TNFRSF agonist or a functional fragment thereof, an IFN agent or a functional fragment of an IFN agent, of the present disclosure may vary according to factors such as the disease stage (e.g., acute vs. chronic), age, sex, medical complications (e.g., HIV co-infection, immunosuppressed conditions or diseases) and weight of the subject, and the ability of the TNFRSF agonist or a functional fragment thereof and IFN agent or a functional fragment of IFN agent to elicit a desired response in the subject. The dosage regimen may be adjusted to provide the optimum therapeutic response. For example, several divided doses may be administered daily, or the dose may be proportionally reduced as indicated by the exigencies of the therapeutic situation.

In general, the compositions provided in the current disclosure may be used to prophylactically treat non-infected cells or therapeutically treat any HBV-infected cells comprising an antigenic marker that allows for the targeting of the HBV-infected cells by a TNFRSF agonist or a functional fragment thereof and an IFN agent or functional fragment of an IFN agent.

Pharmaceutical Compositions and Administration Thereof

Methods of preparing and administering TNFRSF agonists or a functional fragment thereof, IFNs or functional fragments of IFNs, or nucleic acid sequences that encode them, of the current disclosure to a subject are well-known to or can be readily determined by those skilled in the art using this specification and the knowledge in the art as a guide. The route of administration of the TNFRSF agonists or a functional fragment thereof, IFNs or functional fragments of IFNs, or nucleic acid sequences that encode them, of the current disclosure may be oral, parenteral, by inhalation or topical. The term parenteral, as used herein, includes intravenous, intraarterial, intraperitoneal, intramuscular, subcutaneous, rectal or vaginal administration. While all these forms of administration are clearly contemplated as being within the scope of the current disclosure, a form for administration would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumin), etc. In some embodiments, the TNFRSF agonists or a functional fragment thereof, IFNs or a functional fragments of IFNs, or nucleic acid sequences that encode them, can be delivered directly to the site of the adverse cellular population (e.g., the liver) thereby increasing the exposure of the diseased tissue to the therapeutic agent.

In certain embodiments, administration of TNFRSF agonists or a functional fragment thereof, IFNs or functional fragments of IFNs, or nucleic acid sequences that encode them, as described herein is sequential, e.g., administration of a dose of TNFRSF agonist or a functional fragment thereof or a nucleic acid sequence that encodes it, is followed by administration of a dose of IFN agent or a functional fragment of an IFN agent or a nucleic acid sequence that encodes it, or administration of a dose of IFN agent or a functional fragment of an IFN agent or a nucleic acid sequence that encodes it, is followed by administration of a dose of TNFRSF agonist or a functional fragment thereof or a nucleic acid sequence that encodes it.

In certain embodiments, a dose of TNFRSF agonist or a functional fragment thereof and a dose of IFN agent or a functional fragment of an IFN agent, or nucleic acid sequences that encode them, are administered concomitantly, e.g., in separate doses administered close in time, or in the same dose (e.g., as a mixture or as a Duokine).

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the compositions and methods of the current disclosure, pharmaceutically acceptable carriers include, but are not limited to, 0.01-0.1 M, e.g., 0.05 M phosphate buffer, or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like. More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will typically be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof The proper fluidity can be maintained, for example, by the use of a coating such as lecithin, by the maintenance of the required particle size in the case of dispersion and by the use of surfactants.

Prevention of the action of microorganisms can be achieved by various antibacterial and antifungal agents, for example, parabens, chlorobutanol, phenol, ascorbic acid, thimerosal and the like. In many cases, isotonic agents will be included, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a TNFRSF agonist or a functional fragment thereof, an IFN agent or a functional fragments of an IFN agent, or a nucleic acid sequences that encodes any of them, by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, exemplary methods of preparation include vacuum drying and freeze-drying, which yields a powder of an active ingredient plus any additional desired ingredient from a previously sterile-filtered solution thereof The preparations for injections are processed, filled into containers such as ampules, bags, bottles, syringes or vials, and sealed under aseptic conditions according to methods known in the art. Further, the preparations may be packaged and sold in the form of a kit. Such articles of manufacture will typically have labels or package inserts indicating that the associated compositions are useful for treating a subject suffering from HBV infection.

Effective doses of the compositions of the present disclosure, for the treatment of the above described HBV infection-related conditions vary depending upon many different factors, including means of administration, target site, physiological state of the patient, whether the patient is human or an animal, other medications administered, and whether treatment is prophylactic or therapeutic. Usually, the patient is a human, but non-human mammals including transgenic mammals can also be treated. Treatment dosages may be titrated using routine methods known to those of skill in the art to optimize safety and efficacy.

For passive immunization with a TNFRSF agonist or a functional fragment thereof, an IFN agent or a functional fragment of an IFN agent, the dosage can range, e.g., from about 0.0001 to about 100 mg/kg, and more usually about 0.01 to about 5 mg/kg (e.g., about 0.02 mg/kg, about 0.25 mg/kg, about 0.5 mg/kg, about 0.75 mg/kg, about 1 mg/kg, about 2 mg/kg, etc.), of the host body weight. For example dosages can be about 1 mg/kg body weight or about 10 mg/kg body weight or within the range of about 1 to about 10 mg/kg, e.g., at least about 1 mg/kg. Doses intermediate in the above ranges are also intended to be within the scope of the current disclosure. Subjects can be administered such doses daily, on alternative days, weekly or according to any other schedule determined by empirical analysis. An exemplary treatment entails administration in multiple dosages over a prolonged period, for example, of at least six months. Additional exemplary treatment regimens entail administration about once per every two weeks or about once a month or about once every 3 to 6 months. Exemplary dosage schedules include about 1 to about 10 mg/kg or about 15 mg/kg on consecutive days, about 30 mg/kg on alternate days or about 60 mg/kg weekly.

TNFRSF agonists or a functional fragment thereof, IFNs or functional fragments of IFNs, or nucleic acid sequences expressing any of these, can be administered on multiple occasions. Intervals between single dosages can be weekly, monthly or yearly. Intervals can also be irregular as indicated by measuring blood levels of TNFRSF agonists or a functional fragment thereof, IFNs or functional fragments of IFNs in the patient. Alternatively, TNFRSF agonists or a functional fragment thereof, IFNs or functional fragments of IFNs, or nucleic acid sequences expressing any of these can be administered as a sustained release formulation, in which case less frequent administration is required. Dosage and frequency vary depending on the half-life of the TNFRSF agonists or a functional fragment thereof, IFNs or functional fragments of IFNs in the patient.

As previously discussed, a TNFRSF agonist or a functional fragment thereof, an IFN agent or a functional fragment of an IFN agent, of the present disclosure may be administered in a pharmaceutically effective amount for the in vivo treatment of mammalian disorders. In this regard, it will be appreciated that as disclosed a TNFRSF agonist or a functional fragment thereof, an IFN agent or a functional fragment of an IFN agent, will be formulated to facilitate administration and promote stability of the active agent.

A pharmaceutical composition in accordance with the present disclosure can comprise a pharmaceutically acceptable, non-toxic, sterile carrier such as physiological saline, nontoxic buffers, preservatives and the like. A pharmaceutically effective amount of a TNFRSF agonist or a functional fragment thereof, an IFN agent or functional fragment of an IFN agent is an amount sufficient to mediate one or more of: a reduction of HBeAg release from an HBV-infected cell; a reduction of pgRNA transcription in an HBV-infected cell; and a stimulation the IFN agent signaling pathway in an infected cell. Of course, the pharmaceutical compositions of the present disclosure may be administered in single or multiple doses to provide for a pharmaceutically effective amount of the TNFRSF agonist or a functional fragment thereof, IFN agent or functional fragment of IFN agent.

In keeping with the scope of the present disclosure, TNFRSF agonists (e.g., an agonistic antibody directed against a member of the TNFRSF, a soluble TNFRSF agonist including but not limited to its natural ligand), or a functional fragment thereof, IFNs or functional fragments of IFNs, or nucleic acid sequences expressing any of them, may be administered to a human or other animal in accordance with the aforementioned methods of treatment in an amount sufficient to produce a therapeutic effect. The TNFRSF agonists or a functional fragment thereof, IFNs or functional fragments of IFNs, or nucleic acid sequences expressing any of them, can be administered to such human or other animal in a conventional dosage form prepared by combining the TNFRSF agonists (e.g., an agonistic antibody directed against a member of the TNFRSF, a soluble TNFRSF agonist including but not limited to its natural ligand), or a functional fragment thereof, IFNs or functional fragments of IFNs, or nucleic acid sequences expressing any of them, with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of TNFRSF agonists or a functional fragment thereof, IFNs or functional fragments of IFNs, or nucleic acid sequences expressing any of them, described in the current disclosure may prove to be effective.

It is to be understood that the methods described in this disclosure are not limited to particular methods and experimental conditions disclosed herein as such methods and conditions may vary. It is also to be understood that the terminology used herein is for the purpose of describing particular embodiments only, and is not intended to be limiting.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by M R Green and J. Sambrook and Harlow et al., Antibodies: A Laboratory Manual, Chapter 14, Cold Spring Harbor Laboratory, Cold Spring Harbor (2013, 2nd edition).

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present invention has been described with reference to the specific embodiments thereof, it should be understood by those skilled in the art that various changes may be made and equivalents may be substituted without departing from the true spirit and scope of the invention using this disclosure as a guide. Having now described certain embodiments in detail, the same will be more clearly understood by reference to the following examples, which are included for purposes of illustration only and are not intended to be limiting.

### EXAMPLE I

### Synergistic Control of HBV Infection by Cytokines

HBeAg Release

The effect of IFNβ and CD40L on HBV infection in primary hepatocytes was investigated. Primary human hepatocyte (PHH) cells were plated in 24 well plates (˜350 000 cells/well) in William's E GlutaMAX media supplemented with 10% fetal calf serum (FCS), insulin and hydrocortisone+Penicillin/Streptomycin. Four hours later, cells were rinsed and media was changed again the next day. Cells were infected 24 hours later with 1,000 viral genome equivalents (vge)/cell in the presence of 4% PEG 8000. 16 hours post-infection, cells were washed 3 times with PBS. Two days after infection, cells were kept unstimulated, stimulated with IFNα (Pbl assay Science #11100-1, 1000 U/ml), IFNβ (Pbl assay Science #1349872, 100 U/ml), CD40L (Enzo #ALX-522-110, 150 ng/ml) or a combination of CD40L with IFNβ or IFNβ. Four days later, media was removed and cells were stimulated again in the same conditions. Two days after the second stimulation, culture supernatants were collected and kept at −80° C. HBV e antigen (HBeAg) levels in the cell culture supernatant were measured using ELISA as described by the manufacturer and results expressed as National Chinese Unit (HBeAg CLIA 96T/K: CL0312-2 Autobio).

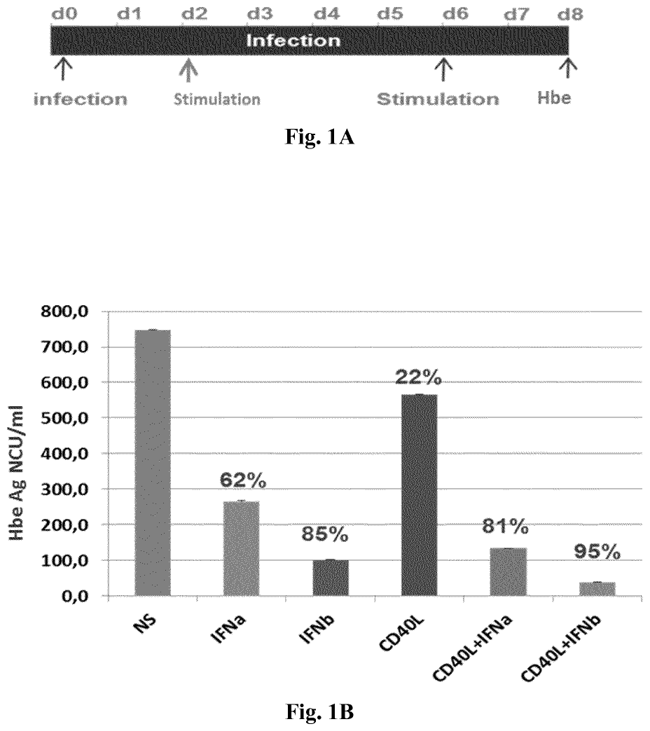

IFNα, used as a reference, reduced the level of secreted Hbe by about 60%. IFNβ was more potent and reduced Hbe released by 85%. Interestingly, CD40L alone was not very active but boosted the effect of either IFNα or IFNβ. The CD40L-IFNβ combination reached 95% inhibition (FIGS. 1A and 1B). Accordingly, a combination of IFNβ and CD40L synergistically reduced HBeAg release.

Transcription of pgRNA

The HBV pre-genomic RNA (pgRNA) is transcribed from the cccDNA followed by reverse transcription to form the relaxed circular DNA (RcDNA) which is encapsidated to form new virions. Thus pgRNA is a key intermediate in the virus life cycle.

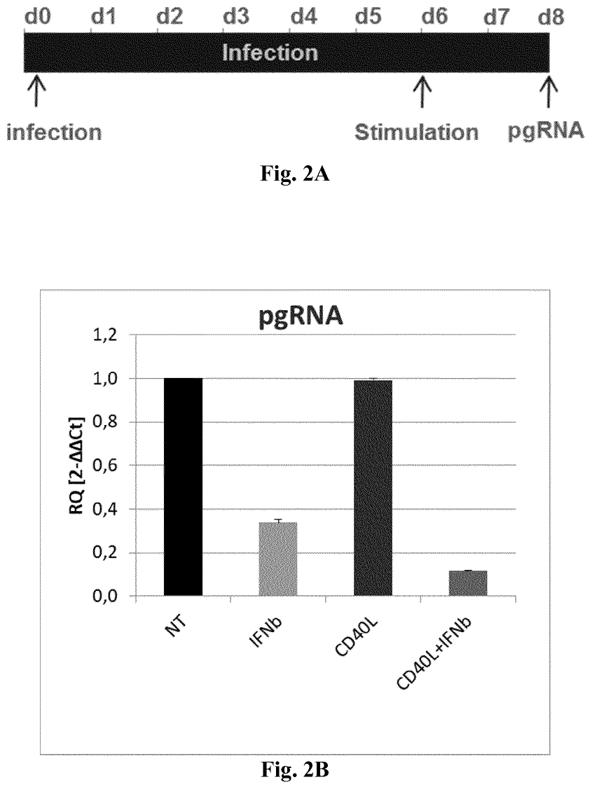

The effect of IFNβ and CD40L on pgRNA transcription from cccDNA was assessed in PHH cells (FIGS. 2A and 2B).

Six days after infection, PHH cells were kept unstimulated, stimulated with IFNb (pblassay #13498723, 100 U/ml), CD40L (Enzo #ALX-522-110, 150 ng/ml) or combination of CD40L and IFNb.

Two days after the stimulation, total RNA was extracted from PHH cells infected with HBV using NucleoSpin® 96 RNA kit (Macherey-Nagel, 740741.4) according to the manufacture's instruction. cDNA templates were synthesized and obtained after reverse transcription using SuperScript® VILO™ cDNA synthesis kit (Invitrogen.) qPCR was performed using the TaqMan® Fast Universal PCR MasterMix (Applied Biosystems) with the following primers and probe for pgRNA: (forward: GCCTTAGAGTCTCCTGAGCA (SEQ ID NO: 34) and reverse: GAGGGAGTTCTTCTTCTAGG (SEQ ID NO: 35) and AGTGTGGATTCGCACTCCTCCAGC (SEQ ID NO: 36) as a probe). The GUSB gene (Hs99999908-m1), was selected as housekeeping gene for the assay. Plates were run on the QuantStudio 12K Flex (Applied Biosystems). Results are expressed in fold change RQ [2-ΔΔCt]. RQ: relative quantification value.

Results indicate that 2 days of treatment with IFNβ was sufficient to reduce by 64% the amount of pgRNA in cells. In these conditions, CD40L alone had no effect. Surprisingly, however, CD40L boosted the effect of IFNβ to reach 88% inhibition.

Synergistic Effect on Signaling Pathways

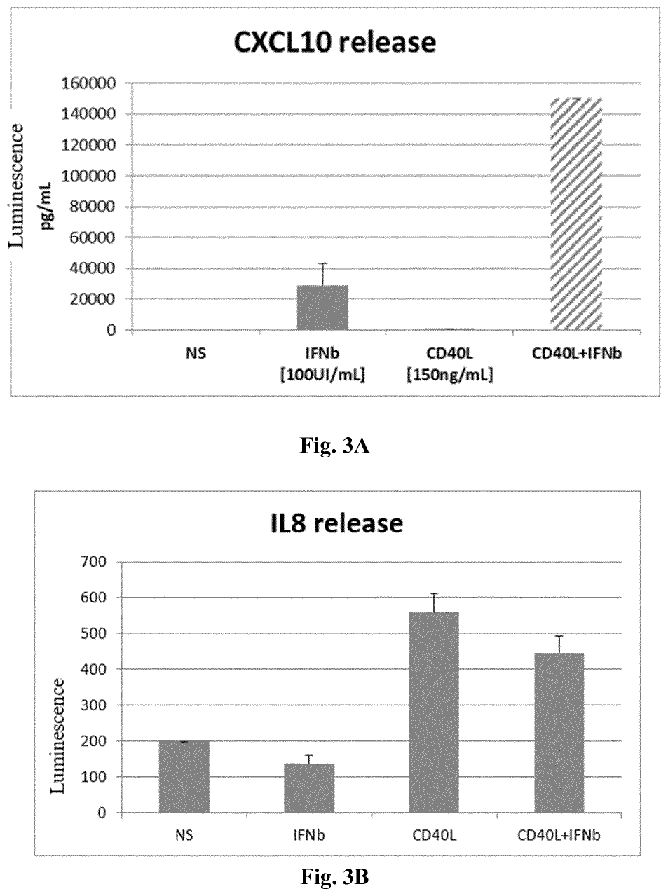

The effect of IFNβ and CD40L on signaling pathways was determined in PHH cells (FIGS. 3A and 3B). Effects on the IFN signaling pathway were assayed by detecting CXCL10 biomarker release (FIG. 3A), and effects on the inflammatory pathway were assayed by detecting IL8 release (FIG. 3B). IFNβ and CD40L were determined to synergistically enhance signaling in the IFN pathway, but did not enhance signaling in the inflammatory pathway.

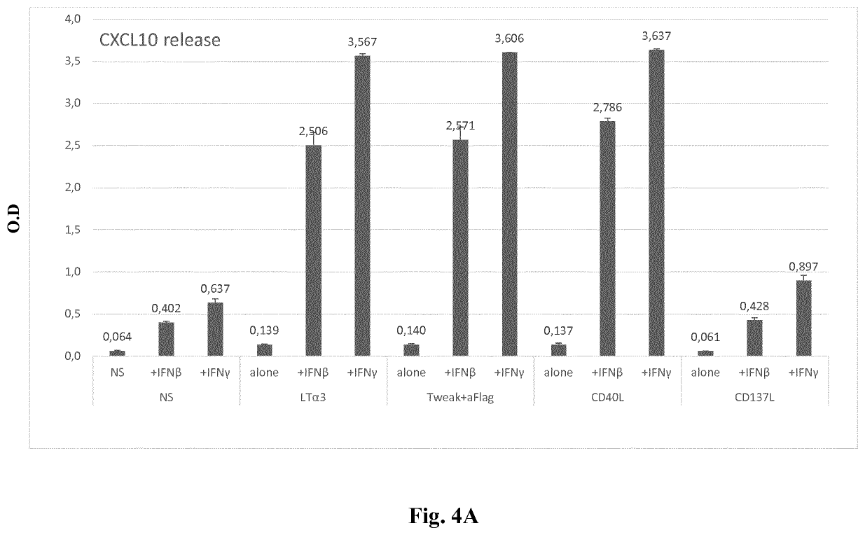

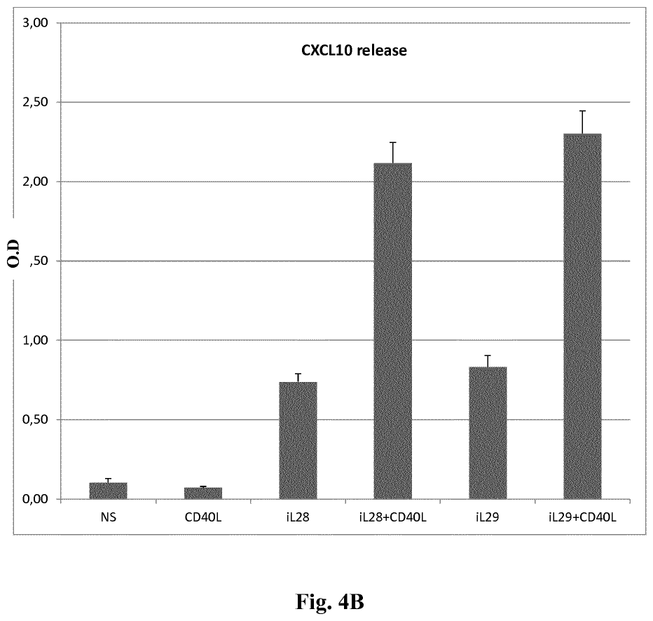

Other TNF family members were assayed with various IFNs (FIGS. 4A and 4B). HepaRG hepatoma cells were seeded in 24 well plates (approximately 250,000 cells/well) in William's E GlutaMAX media supplemented with 10% FCS, insulin and hydrocortisone+P/S. Six hours later, cells were rinsed and kept unstimulated or stimulated overnight with the indicated cytokines (TWEAK (Enzo, ALX-522-021, 1 μ/ml final)+mouse monoclonal Anti-Flag-M2 (Sigma, F-3165, 1 μg/ml final), LTα3 (Enzo, ALX-522-034, 1 μ/ml final), CD40L (Enzo #ALX-522-110, 150 ng/ml final), CD137L (Enzo, ALX-522-111, 1 μ/ml final); IFNβ-1a (pblassay #13498723, 100 U/ml final), IFNγ (Millipore #IF-002, 20 ng/ml), IL28 and IL29 R&D). Supernatant was then collected and CXCL10 was quantified by ELISA as described by the manufacturers (BioLegend 439904).

Results indicated that like CD40L, LTα3 and Tweak (but not 4 1-BB/CD137L which is a negative control because its receptor is not expressed) synergistically enhanced CXCL10 release induced not only by IFNβ but also by the Type II interferon (IFNγ) (FIG. 4A). In addition, costimulation of hepatocytes with CD40L and Type III interferons (IL28 and IL29) was also able to synergistically boost the interferon pathway (FIG. 4B).

Together, these results indicate that the synergy results from the activation of pathways common to the TNFRSF and to the IFN Receptors.

CD40 could be stimulated in different manners, by soluble CD40L (a trimeric form—1 μg/ml), by membrane CD40L (which is mimicked here by the hexameric form of CD40L—1 μg/ml) and also by agonistic anti-CD40 antibodies (CP-870,893, Pfizer/Roche—1 μg/ml).

To understand whether the synergy could be induced, HepaRG cells were kept unstimulated or stimulated overnight with the indicated cytokines or antibody either alone or in the presence of IFNβ. Supernatant was then collected after overnight stimulation and CXCL10 quantified by ELISA as described earlier.

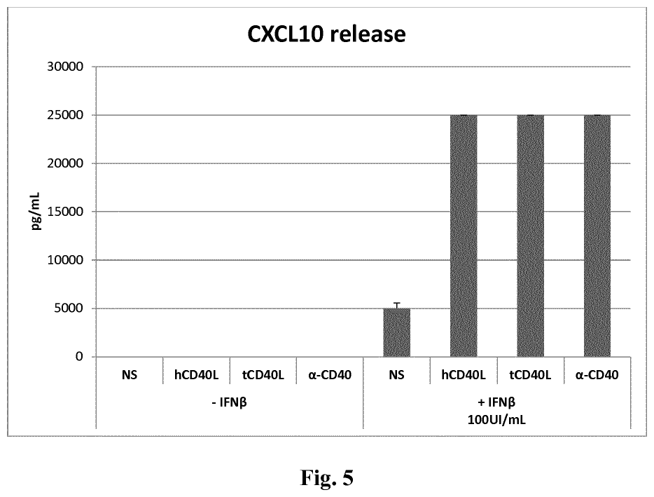

Results indicated that, except for IFNβ, none of the stimuli were able to induce CXCL10 as a single agent. However, addition of IFNβ to trimeric CD40L, hexameric CD40L, or an agonistic anti-CD40 antibody boosted CXCL10 release (FIG. 5). These results show that several mechanisms to activate CD40 lead to enhancement of the IFNβ pathway.

The synergistic effects of CD40L and IFNβ may have varying effects on different signaling pathways. HepaRG hepatoma cells were seeded in 24-well plates (approximately 250,000 cells/well) in William's E GlutaMAX media supplemented with 10% FCS, insulin and hydrocortisone+P/S. Six hours later, cells were rinsed and kept unstimulated or stimulated with an effective dose of CD40L in the presence or absence of IFNβ (100 U/ml). CXCL10 and IL8 and were quantified by ELISA from supernatants collected after overnight stimulation. Quantification of CXCL10 and IL8 was performed as described by the manufacturers. For CXCL10: BioLegend 439904—For IL8: Ready-Set ELISA human iL8: eBiosciences Ref 88-8086-88.

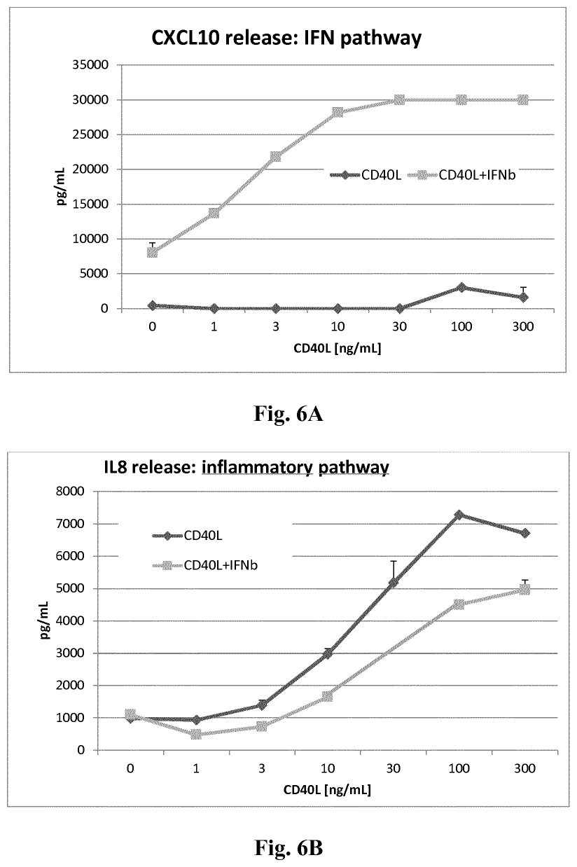

Results indicated that CD40L boosted the IFN pathway, starting at 1 ng/ml of CD40L (FIG. 6A). Interestingly, IL8, which is induced by CD40L in a dose-dependent manner, was not boosted but rather reduced by IFNβ (FIG. 6B). These results indicate that co-stimulation of hepatocytes with CD40L and IFNβ synergistically boosts the IFN-pathway while reducing the release of the inflammatory cytokine IL8.

The synergy of CD40L and IFNβ was further assayed to determine if synergy occurred at the mRNA level or at the post-translational level, and whether other interferon stimulated genes were also boosted by the combination (other than CXCL10).

For this purpose, RNAseq analysis was performed in HepaRG cells. HepaRG cells were stimulated with CD40L, IFNβ or a combination of both cytokines in a kinetic manner from 30 minutes to 24 hours. Cells were collected, washed and pelleted. Cell pellets were lysed by addition of QIAzol buffer. RNA was extracted with an RNeasy kit (Qiagen), quantified by Xpose (ng/μl) and quality, integrity (RIN) was controlled with by an Agilent bioanalyzer. Libraries were produced using the TruSeq Stranded Total RNA LT (with Ribo-Zero Gold) kit from Illumina and RNA sequencing was done on the NextSeq 500 apparatus using the NextSeq 500 High Output Kit.

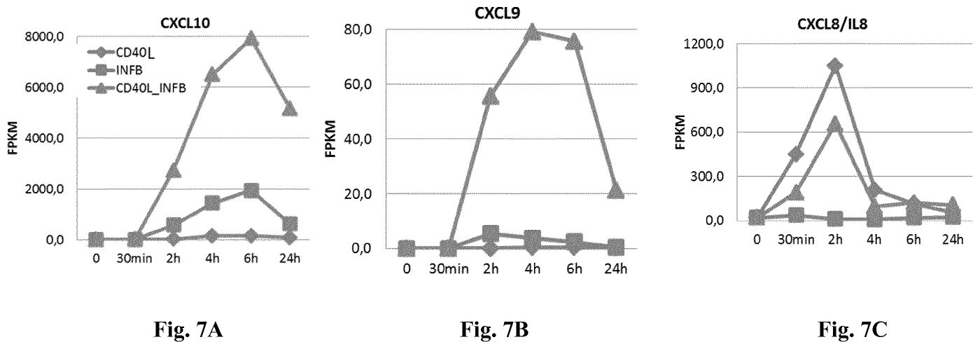

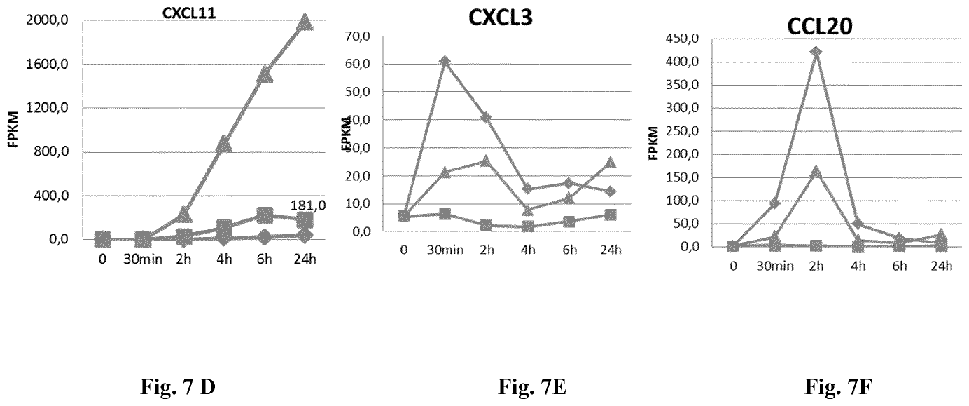

Analysis of CXCL10 mRNA indicated that its expression was induced by IFNβ and was synergistically induced by the combination starting at 2 hours post-stimulation (FIG. 7A). Interestingly, the synergistic effect was observed for other interferon stimulated genes such CXCL9 and CXCL11 (FIGS. 7C and 7D).

Analysis of CXCL8/IL8 mRNA indicated that it was only induced by CD40L, and the combination reduced its expression (FIG. 7B). This reduction was also observed with other chemokines induced by CD40L, such as CXCL3 or CCL20 (FIGS. 7E and 7F).

Together, these results confirm that co-stimulation of hepatocytes with CD40L and IFNβ boost the IFN pathway but not the CD40L-induced inflammatory pathway.

The boost of the IFN pathway was also observed in PHH cells. Cells were plated in 96 well plates (approximately 350,000 cells/well) in William's E GlutaMAX media supplemented with 10% FCS, insulin and hydrocortisone+P/S. 24 hours later, cells were rinsed, media was changed, and Matrigel was added. The next day, cells were infected or not with 1,000 viral genome equivalents (vge)/cell in the presence of 4% PEG 8000. 16 hours post-infection, cells were washed 3 times with PBS, were kept unstimulated (NS), stimulated with IFNβ (pbl assay #1349872, 100 U), CD40L (Enzo #ALX-522-110, 150 ng/ml) or a combination of CD40L and IFNβ. 3 days later (d3), media was collected and cells were stimulated again in the same conditions. 4 days later after the second stimulation (d7) culture supernatants were collected again and CXCL10 release was assessed by AlphaLISA hCXCL10: Perkin Elmer: AL259F.

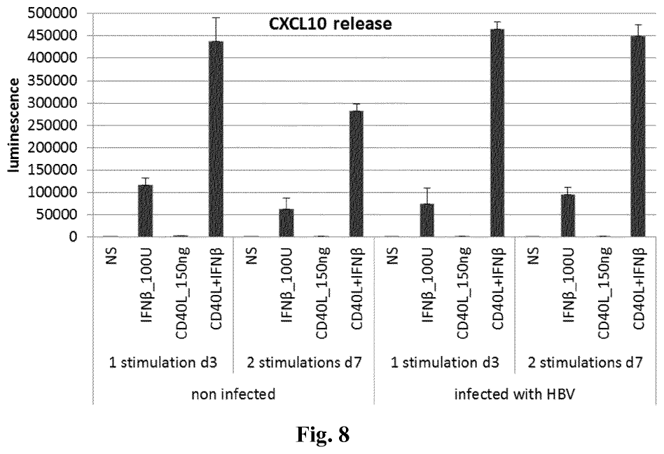

Results indicated that IFNβ, but not CD40L, induced CXCL10 in primary hepatocytes, and costimulation with CD40L ligand boosted its release after one or two stimulations and either in HBV-infected or non-infected cells (FIG. 8).

TNF family members other than CD40L may work synergistically with IFNβ. Constructs were designed to express the extracellular part of human Tweak or human LIGHT fused with the Fc-region of human IgG1 (Ig-Tweak, SEQ ID NO: 18 and Ig-Light, SEQ ID NO.19, respectively). These fusions were cloned into an expression vector. After transfection in HEK cells, supernatants were collected and used to stimulate HepaRG cells as previously described. As controls, cells were transfected with an empty vector or with a plasmid encoding for IFNβ. After overnight stimulation, supernatants were collected for CXCL10 assessment. Results indicated that IFNβ alone, but not LIGHT or TWEAK alone, was able to induce CXCL10 release. This release was boosted when cells were co-stimulated with IFNβ and TWEAK (FIG. 9A) or LIGHT (FIG. 9B). These results also indicated that such combinations could also play an important role in synergistically boosting the anti-HBV innate immune response in hepatocytes.

To generate proteins to be used to further demonstrate the synergistic effects of TNFR agonism and IFN treatment on HBV infected cells, Ig-Tweak (SEQ ID NO:18) and Ig-Light (SEQ ID NO:19) were produced. After purification on protein A columns, the activity of these proteins was evaluated in a dose effect on A549 dual cells (InvivoGen, Catalog #a549d-nfis), which naturally express the receptors LTbR and TweakR. These cells are designed to monitor the activation of the NFkB pathway by quantifying the activity of the reporter gene SEAP, secreted in the supernatant, using QUANTI-Blue™ (a SEAP detection reagent (QUANTI-Blue™ rep-qbl InvivoGen)). Results indicate that both molecules activate the NFkB pathway in a dose dependent manner (FIG. 9C and FIG. 9D).

Ig-Tweak (SEQ ID NO:18) and Ig-Light (SEQ ID NO:19) proteins were then evaluated in combination with IFNb on primary hepatocytes infected by HBV, as described earlier. Cells were infected, kept untreated (NT), treated with IFNb (100 U), Ig-Light (1 μg/ml) or Ig-Tweak (1 μg/ml) or with a combination IFNb (100 U)+Ig−Light (1 μg/ml) or IFNb (100 U)+Ig−Tweak (1 μg/ml). Results show that both Ig-Tweak (FIG. 9E) and Ig-Light (FIG. 9F) were able to enhance IFNb induced anti-viral effect.

The use of a single molecule to illicit the synergistic effects of the combination of CD40L and IFNβ was investigated. To this end, three Duokines were designed and cloned into pCDNA3. IFNβ (at the N-terminus) was linked to the extracellular domain of CD40L (C-terminus) by a linker comprising the Fc part of human IgG1 (IFNβ-Ig-CD40L or SEQ ID NO: 9), using a Gly-Ser-Thr linker (IFNβ-CD40L or SEQ ID NO: 10), or a linker comprising a leucine zipper domain (IFNβ-LZ-CD40L or SEQ ID NO: 11) (See Table 3). The constructs were transiently transfected in HEK cells. 48 hours after transfection, the supernatant was collected and added on HEK-Blue™ CD40L cells (InvivoGen Cat. #: hkb-cd40) or HEK-Blue™ IFN-α/β cells (InvivoGen, Cat. #: hkb-ifnab). These cells are specifically designed to monitor, respectively, the activation of the NFκB pathway by CD40L or of the JAK-STAT pathway induced by type I IFNs. After stimulation, the expression of the reporter gene SEAP (under the control of NFκB or JAK/STAT promoters) was detected. SEAP was secreted in the supernatant and detected using QUANTI-Blue™ (a SEAP detection reagent (QUANTI-Blue™ rep-qb1 InvivoGen)).

Results indicated that HEK-Blue-CD40L reporter cells (FIG. 10A) but not HEK-Blue-IFN reporter cells (FIG. 10B) were able to respond to recombinant CD40L. Only HEK-Blue-IFN reporter cells respond to IFNβ. Interestingly, stimulation of these cells with media collected from HEK transfected cells indicated that all Duokines were able to stimulate both reporter cells. As negative controls, supernatant from cells transfected with empty vector did not stimulate cells, and supernatant from cells transfected with IFN13 was only able to stimulate HEK-Blue-IFN reporter cells.

The results indicated that CD40L-IFNβ fusions, regardless of linker, were still active on their respective receptors.

The Duokine molecules were then tested on non-reporter hepatocytes (HepaRG) cells and CXCL10 release was assessed. To this end and in addition to the three Duokines mentioned above, two other Duokines were designed and cloned into pCDNA3. IFNβ (at the N-terminus) was linked to the extracellular domain of TWEAK (C-terminus) by a linker comprising the Fc part of human IgG1 (IFNβ-Ig-TWEAK or SEQ ID NO: 12), IFNβ (at the N-terminus) was linked to the extracellular domain of LIGHT (C-terminus) by a linker comprising the Fc part of human IgG1 (IFNβ-Ig-LIGHT or SEQ ID NO: 15), or by a linker comprising a leucine zipper (IFNβ-LZ-LIGHT or SEQ ID NO: 17) (See Table 3). The supernatants collected from HEK transfected cells were used to stimulate hepatocytes (HepaRG) cells. After overnight stimulation, CXCL10 was assessed in the supernatant by ELISA. Results indicate that all Duokines are able to induce CXCL10 release and this induction is enhanced in comparison to stimulation with IFNβ alone (FIGS. 11A, 12 and 13). Interestingly, when stimulation was performed in the presence of an anti-CD40L antagonistic antibody (mabg-h401-3, InvivoGen) which neutralizes the action of CD40L, CXCL10 release was highly reduced (FIG. 11B). This confirms that CD40L within the Duokine is still able to synergize with IFNβ to boost the IFN pathway.

To assess the effect of a fusion protein comprising type-I interferon and CD40L, a duokine encoding IFNα (amino terminus) and CD40L (carboxy terminus) linked by a linker comprising the hu-IgG1-Fc part (IFNa-huIgG1-hu-CD40L; SEQ ID NO: 25) was constructed. HEK cells were transiently transfected and proteins purified on protein A columns. Proteins were evaluated on HEK-Blue™ CD40L cells (FIG. 19A; InvivoGen Cat. #: hkb-cd40) or HEK-Blue™ IFN-α/β cells (FIG. 19B; InvivoGen, Cat. #: hkb-ifnab) as described previously. Results indicate that the duokine is able to induce SEAP release in a dose dependent manner in both CD40 (FIG. 19A) and IFNa/b (FIG. 19B) reporter cells, demonstrating that both CD40L and IFN domains are active.

The duokine was then evaluated on primary human hepatocytes infected by HBV (FIG. 19C), as described earlier. Cells were infected, and then kept untreated (NS), treated with recombinant IFNα (pblassay #11100-1, 100 U), megaCD40L (Enzo #ALX-522-110, 100 ng/ml) or with a combination of both molecules. In parallel, cells were treated with the duokine in a dose effect. Results show that CD40L boost IFNα induced anti-viral effect and that duokine is highly active on HBV infection as it reduced Hbe release in a dose dependent manner with an IC50 around 3 ng/ml.

### EXAMPLE II

### Human Liver Chimeric Mouse Models

As the only natural cell target of HBV infection and replication is the human hepatocyte, the human liver chimeric mouse model is suitable for studying HBV infection in vivo and for evaluating direct anti-viral and hepatocyte-directed host agents (Dandri et al. Best Pract Res Clin Gastroenterol. 2017 Jun. 31(3):273-279). Such a model is based on two requirements: 1) endogenous murine hepatocytes are damaged to create the space for the transplanted hepatocytes to reconstitute the mouse liver, and 2) the host immune response is abolished to allow survival of the transplanted xenogeneic hepatocytes.

Various chimeric mice have been developed to study HBV pathogenesis and potential therapies (Giersch K et al. Sci Rep. 2017 Jun. 16;7(1):3757; Tsuge et al. Virus Antimicrob Agents Chemother. 2017 June; 61(6): e00183-17; Bissig et al. J Clin Invest. 2010 March; 120(3):924-30; and Kosaka et al. Biochem Biophys Res Commun. 2013 Nov. 8; 441(1):230-5). uPA-SCID mice transplanted with human hepatocytes are commercially available (KMT Hepatech, Inc., Edmonton, Canada).

Human liver chimeric mouse models can be used to test the IFNβ-CD40L described herein. Human liver chimeric mouse models have several useful features. The uPa-SCID mice are highly immunodeficient, with mouse urokinase-type plasminogen activator (uPA) gene under the control of the mouse albumin enhancer/promotor (Giersch K et al. and Tsuge et al.). FRG mice are highly immunodeficient, having fumarylacetoacetate hydrolase knock-out Fah-/- (Bissig et al.). The TK-NOG mice are highly immunodeficient and express a Herpes Simplex Virus-1 Thymidine Kinase (HSVtk) transgene driven by the mouse albumin enhancer/promoter in its liver) (Kosaka et al.).

Soon after birth for uPA-SCID mice or after murine hepatocyte damage induction (2-(2-nitro-4-trifluoro-methyl-benzoyl)-1, 3 cyclohexanedione (NTBC)-based removal for FRG mice or Ganciclovir injection for TK-NOG mice), intra-splenic injection of primary human hepatocytes is performed for human hepatocyte liver repopulation.

The level of chimerism can be monitored by determining human serum albumin (HSA) and/or human α1-antitrypsin (hAAT) levels, which can range from 20% to 70% depending on the model and the inter-individual variability. After a period of 6 to 8 weeks, the mice can be infected with natural HBV and the full viral life cycle is engaged, including the entry, the cccDNA formation, and the replication and spreading of the virus. This human liver chimeric mouse model also allows the study of the direct anti-viral effects, because of the immune deficient status of these mice, of the human drugs, using various viral read-outs, including cccDNA levels and modulation.

### EXAMPLE III

### Chimeric AAV/HBV Virus Model

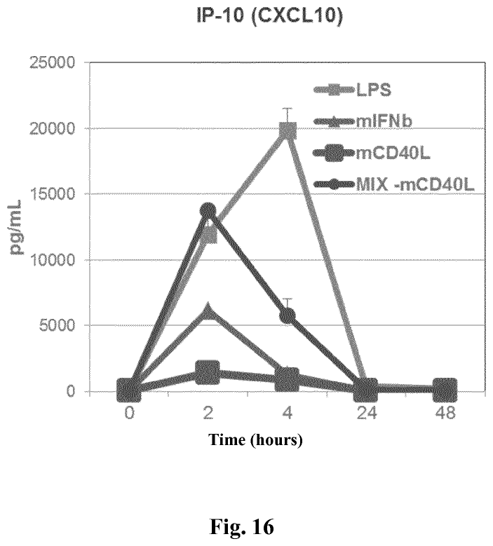

The synergistic effects of CD40L in combination with IFNβ was determined in an in vivo mouse model by assaying CXCL10 release into serum. To that purpose, C57/B6J mice were injected either with 10⁶ Units of murine IFNβ (8234-MB/CF), 100 μg of CD40L (ALX-52-120-000) or their combination. A group of mice were injected with 50 μg of LPS as a positive control. This demonstrates that when administered in vivo in non-infected mice, the combination synergistically boosted the IFN pathway as shown on CXCL10 plasmatic level (FIG. 16). CXCL10 will be used as a pharmacodynamic biomarker to define the optimal treatment regimen (doses and frequency of administration).

AAV/HBV-transduced mice are based on viral vector-mediated HBV entry into murine hepatocytes. AAV viral particles are injected intravenously into immunocompetent mice, and the particles deliver the HBV genome directly into the hepatocytes. Many infected cells express the HBV c antigen (HBc), reflecting that HBV replication occurs in these cells. This viral replication is engaged early after the infection as demonstrated by the HBV e and s antigen levels (HBe and HBs) in plasma that are already high and at a plateau 21 days post-infection. The level of circulating HBV DNA is also very high, reflecting the virion production. This high and stable replication stage recapitulates the immune tolerant phase observed in patients (Dion et al. (2013) J Virol. May; 87(10):5554-63 and Yang et al. (2014) Cell Mol Immunol. Jan; 11(1):71-8). Recent work demonstrated that all viral intermediates are present in this model, including the cccDNA production, as shown on the southern blot (Lucifora et al. (2017) Antiviral Res. Sep; 145:14-19). The quantification of this cccDNA remains difficult and protocols are still under optimization. Id.

The immunocompetent AAV/HBV model described here allows for the study of both immune-modulation and direct anti-viral effects of the IFNβ-CD40L combination. In that case, the read-outs will be viral parameters and also specific HBV Ag-Antibody production, which are the same parameters used in the clinic.

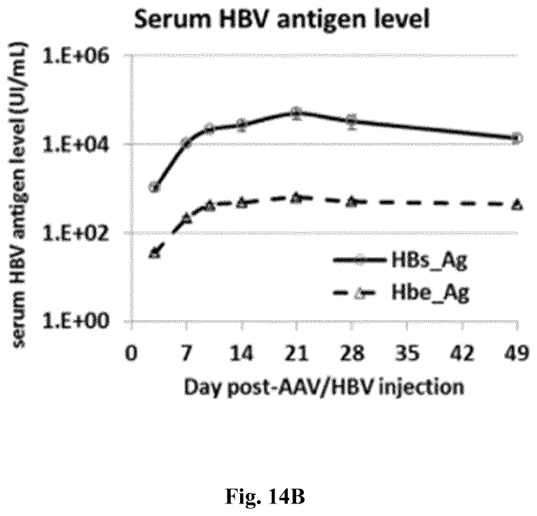

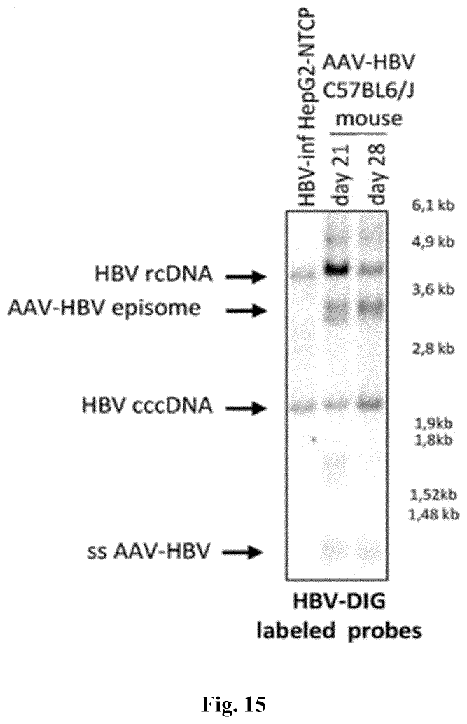

A standard protocol is as follows. All in vivo experiments are made according to French and European regulations on animal welfare and Public Health Service recommendations and all protocols are reviewed and approved by the institutional animal care committee of Sanofi. All animals are housed in a specific-pathogen-free environment in the animal facilities of Sanofi, Marcy l'Etoile, France. Eight-week-old C57BL6/J female mice (Charles River, Les Oncins, Saint-Germain Nuelles, France) receive an intravenous injection of 5×10¹° viral genomes/mouse of AAV8-HBV viral particles. After 28 days post-injection, mice are randomly assigned, using HBs-Ag plasma levels, into the different treatment cohorts. During the treatment period, weekly blood collections are performed for circulating viral parameter monitoring (HBV-Ag and DNA). (See FIGS. 14A-14B and 15.) After the treatment period, usually between 2 to 4 weeks, mice are euthanized, blood is collected and liver pieces are flash frozen in liquid nitrogen and kept at −80° C. before further processing. HBV Antigen levels (HBs, Hbe) are evaluated by ELISA (AutoBio kit according to the manufacturer's instructions (AutoBio, China)). Viral load in the serum and the liver are evaluated using PCR. The level of HBV pgRNA in the liver is measured by qRT-PCR. DNA extraction from liver biopsies, followed by a triple digestion (Xmal, Xhol, and T5 enzyme) as described by Lucifora et al. would allow for cccDNA quantification.

Depending on the model, AAV/HBV or HuHep mice, four to eight weeks after infection of the mice with HBV, mice will be treated 3 times a week with vehicle, IFNβ, CD40L (or agonistic antibody anti-CD40, FKG450) and a combination of both agents. Blood samples will be collected once a week and at the endpoint, blood and liver tissues will be collected. Different readouts will be assessed, including HBV parameters (circulating Hbe Ag and Hbs Ag, cccDNA and pgRNA in the liver) as well as host parameters including cytokine release, seroconversion, and liver enzymes (e.g., AST and ALT).

### EXAMPLE IV

### Chimeric AAV/HBV Virus Model

To perform the in vivo experiment, murine tools compatible with chronic treatment were generated. mIFNb-Fc-mIgG1 molecule (SEQ ID NO: 20) and mIgG1-Fc-mCD40L (SEQ ID NO: 21) as well as Fc-mIgG1 (SEQ ID NO: 22) molecules were designed and cloned into an expression vector. After transient overexpression, proteins were purified using protein A columns and then tested in a dose dependent manner on reporter cells to demonstrate their activity. mIFNb-Fc-mIgG1 was tested on RAW-Dual™ IRF (IFN pathway) and MIP-2 (NF-kB) reporter mouse macrophages (Invivogen, Catalog #rawd-ismip). After stimulation, the expression of the reporter gene lucia (under the control of JAK/STAT promoters) was detected. Lucia was secreted in the supernatant and detected using QUANTI-luc™ (a lucia detection reagent (QUANTI-Luc™ rep-qlcl InvivoGen)). Results show that mIFNb-Fc-mIgG1 was active and induced in a dose dependent manner the IRF pathway activation (FIG. 17A). Fc-mIgG1-mCD40L molecule was tested on HEK-Blue-CD40 cells as described previously. Results show that Fc-mIgG1-mCD40L was active and induced in a dose dependent manner SEAP release (FIG. 17B).

To demonstrate their respective bioavailability, 0.84 μg of mIFNb-Fc-mIgG1 and 30 μg of Fc-mIgG1-mCD40L were administrated IP and blood samples collected at different time points. Circulating mIFNb-Fc-mIgG1 was quantified using an ELISA kit according to the manufacturer's instructions (Verikine Mouse IFNb Elisa Kit, 42400-1). For the quantification Fc-mIgG1-mCD40L: 96-wells plates were coated overnight at 4° C. with 100 μl of Recombinant Mouse CD40/TNFRSF5 Fc Chimera Protein, consisting of the extracellular domain of murine CD40 fused to the Fc part of human IgG1 (rmCD40-Fc; 215-CD-050, R&D Systems) at 0.1 μg/ml in Carbonate Sodium (0.05M, pH=9.6, C-3041, Sigma). After emptying by flipping, plates were then incubated during 1 hours at 37° C. with PBS—0.05% Tween20—1% Milk followed by washing with PBS—0.05% Tween20. Samples and controls (100 μ1 of 1/10 dilutions) were then incubated during 90 minutes at 37° C. followed by 3 washes (PBS—0.05% Tween20) and incubation with a secondary anti-mouse IgG1(1/20 000, ab97240, Abcam) antibody (in PBS·0.05% Tween20—1% Milk). After 3 washes with PBS—0.05% Tween2, TMB (Tetramethylbenzidin) was added and the plates incubated for 20 mins in the dark. The reaction was stopped by adding HCl 1N. Plates were read at 450-650 nm with an Ensight (Perkin Elmer).

Pharmacokinetic analysis indicated that both mIFNb-Fc-mIgG1 molecule (FIG. 17C) and mIgG1-Fc-mCD40L (FIG. 17D) circulated in the blood for at least 6 h post administration. Both molecules circulated with a detectable level at least 6 h after administration (FIGS. 17 C&D).

For in vivo evaluation, mice received an intravenous injection of 5.10\hat{ }10 genome equivalent/mouse of AAV8-HBV viral particles or PBS as control for non-infected animals. After 14 days post-injection (dpi), mice were randomly assigned into the different treatment cohorts, using peripheral blood viral parameters (HBV DNA and Antigens) level. Five groups were created with an total of 12 mice for the non-infected group and 18 mice for each treatment in infected groups as follow: Non-infected Fc_mIgG1 (45 μg/kg); AAV/HBV_Fc-mIgG1 (45 μg/kg); AAV/HBV Fc-mIgG1 (0,25 μg/kg); AAV/HBV_Fc-mIgG1-mCD40L (45 μg/kg); AAV/HBV_Combi (mIFN-Fc-mIgG1 (0,25 μg/kg)+Fc-mIgG1 -mCD40L (45 μg/kg). Treatments were given twice a week by intraperitoneal injection (10 mL/kg). After 2 weeks and 4 weeks of treatment, respectively at 28 and 42 dpi, some mice from each groups were euthanized and blood, liver and spleen were collected. Serum, plasma and liver pieces were flash frozen in liquid nitrogen and kept at −80° C. before further processing. HBV Antigen level (Hbe-Ag) was evaluated by ELISA (Autobio kit according to the manufacturer's instructions (AutoBio, China)). Viral load in the serum and the liver were evaluated using ddPCR techniques. The level of HBV pgRNA in the liver was measured by qRT-PCR.

AAV/HBV viral particles injection led to high and stable HBV virus expression as shown by the follow-up of peripheral blood Hbe-Ag and HBV DNA levels (FIGS. 18B and C) as well as HBV nucleic acids in the liver tissue (HBV DNA and pgRNA—FIGS. 18D and E).

mIFN-Fc-mIgG1 and Fc-mIgG1 -mCD40L given alone, twice weekly, reduced HBV DNA level both in the peripheral blood and the liver tissue (FIGS. 18C and D). No effect of these molecules alone was detected on viral protein HBe-Ag or HBV pgRNA level (FIGS. 18B and E).

The combination of mIFN-Fc-mIgG1 and Fc-mIgG1-mCD40L demonstrated a synergistic reduction effect on viral load read-outs as shown by the strong decrease of peripheral blood and the liver tissue HBV DNA (FIGS. 18C and D). In addition, the combination achieved a drop in Hbe-Ag secretion and pgRNA expression (FIGS. 18B and E).

### Equivalents

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

1. A method of treating a HBV infection in a subject in need thereof, comprising administering to the subject a combination of a tumor necrosis factor receptor superfamily (TNFRSF) agonist or a functional fragment thereof and an interferon (IFN) agent or a functional fragment thereof.

2. The method according to claim 1, wherein the TNFRSF agonist or a functional fragment thereof is selected from the group consisting of a lymphotoxin alpha 3 receptor agonist, a lymphotoxin beta receptor agonist, a herpesvirus entry mediator agonist, a tumor necrosis factor-like receptor weak inducer of apoptosis agonist, a cluster of differentiation factor 40 agonist, a CD27 agonist, a CD30 agonist, a 4-1BB agonist, a receptor activator of nuclear factor κB agonist, a Troy agonist, and a OX40 receptor agonist, or functional fragments thereof.

3. The method according to claim 1, wherein the TNFRSF agonist or a functional fragment thereof is selected from the group consisting of CD40L, LTα3, LIGHT and TWEAK, or functional fragments thereof.

4. The method according to claim 1, wherein the TNFRSF agonist or a functional fragment thereof is selected from the group consisting of CD40L, LIGHT and TWEAK, or functional fragments thereof.

5. The method according to claim 1, wherein the TNFRSF agonist or a functional fragment thereof is a CD40 agonist or a functional fragment thereof selected from the group consisting of a CD40 ligand (CD40L) or a functional fragment thereof, an agonistic anti-CD40 antibody, a functional fragment thereof or antigen-binding fragment thereof, and a fusion protein comprising a CD40 ligand or a functional fragment thereof.

6. The method according to claim 5, wherein the CD40L is hexameric CD40L or trimeric CD40L.

7. The method according to claim 1, wherein the TNFRSF agonist or a functional fragment thereof is a polypeptide or a functional fragment thereof, an antibody or a functional fragment thereof, or an antigen-binding fragment thereof.

8. The method according to claim 1, wherein the TNFRSF agonist or a functional fragment thereof is provided as a fusion protein comprising said TNFRSF agonist or a functional fragment thereof.

9. The method according to claim 1, wherein the IFN agent or a functional fragment thereof is selected from the group consisting of a Type I IFN agent, a Type II IFN agent and a Type III IFN agent, or functional fragments thereof.

10. The method according to claim 1, wherein the IFN agent or a functional fragment thereof is IFNα, IFNβ, IFNγ or IFNλ, or functional fragments thereof.

11. The method according to claim 1, wherein the IFN agent or a functional fragment thereof is IFNβ or IFNγ, or functional fragments thereof.

12. The method according to claim 1, wherein the IFN agent or a functional fragment thereof is IFNβ, or a functional fragment thereof.

13. The method according to claim 1, wherein the IFN agent or a functional fragment thereof is IFNα, or a functional fragment thereof.

14. The method according to claim 1, wherein the IFN agent or a functional fragment thereof is provided as a fusion protein comprising said IFN agent or a functional fragment thereof.

15. The method according to claim 1, wherein the TNFRSF agonist or a functional fragment thereof and the IFN agent or a functional fragment thereof are provided as a bifunctional immunostimulatory fusion protein comprising the said TNFRSF agonist or a functional fragment thereof, the said IFN agent or a functional fragment thereof and a linker.

16. The method according to claim 1, wherein the TNFRSF agonist or a functional fragment thereof and the IFN agent or a functional fragment thereof are comprised in a single pharmaceutical composition.

17. The method according to claim 1, wherein the TNFRSF agonist or a functional fragment thereof and the IFN agent or a functional fragment thereof are comprised in distinct pharmaceutical compositions.

18. A pharmaceutical composition comprising:
a TNFRSF agonist or a functional fragment thereof; and
a Type II IFN agent, a functional fragment of a Type II IFN agent, a Type III IFN agent, or a functional fragment of a Type III IFN agent.
